(19) United States
(12) Patent Application Publication
(10) Pub. No.: US 2024/0158564 A1
(43) Pub. Date: May 16, 2024

(54) QUATERNARY AMMONIUM-FUNCTIONALIZED POLYMERS AND METHODS OF MAKING AND USING SAME

(71) Applicant: Cornell University, Ithaca, NY (US)

(72) Inventors: Geoffrey W. Coates, Lansing, NY (US); Wei You, Beijing (CN); Cheyenne Peltier, Ithaca, NY (US)

(21) Appl. No.: 18/262,677

(22) PCT Filed: Jan. 24, 2022
(86) PCT No.: PCT/US22/13579

§ 371 Date: Jul. 24, 2023

Related U.S. Application Data
(60) Provisional application No. 63/140,827, filed on Jan. 23, 2021.

(51) Int. Cl. C08G 61/08; B01J 41/05; B01J 41/14; B01J 47/12; C07D 295/037; H01M 8/103

(52) U.S. Cl. CPC C08G 61/08; B01J 41/05; B01J 41/14; B01J 47/12; C07D 295/037; H01M 8/103; C08G 2261/122; C08G 2261/143; C08G 2261/418; C08G 2261/516; H01M 2008/1095

(57) ABSTRACT

Hydrocarbonbackbone polymers with pendant quaternary ammonium groups and methods of making and using same. A polymer can be made by a ring-opening polymerization of quaternary ammonium bearing monomer(s), and, optionally, non-quaternary ammonium bearing monomer(s). A film including a polymer can be used as an anion exchange membrane in a device, such as, for example, a battery, a fuel cell, or the like.

### CROSS-REFERENCE TO RELATED APPLICATIONS

This application claims the benefit of U.S. Provisional Patent Application No. 63/140,827, filed Jan. 23, 2021, the contents of the above-identified application are hereby fully incorporated herein by reference in their entirety.

### STATEMENT REGARDING FEDERALLY SPONSORED RESEARCH

This invention was made with government support under grant no. DE-SC0019445 awarded by the U.S. Department of Energy and grant no. GM134893 awarded by the National Institutes of Health. The government has certain rights in the invention.

### BACKGROUND OF THE DISCLOSURE

The replacement of fossil fuels with renewable fuels (e.g., H₂) will be an important part of addressing issues related to CO₂ emission. Anion exchange membrane fuel cells (AEMFCs) are a state-of-the-art technology that efficiently convert chemical energy to electricity without the use of expensive Pt-group electrocatalysts. The alkaline anion exchange membrane (AAEM) and the anion exchange ionomer (AEI), which are both composed of polymeric backbones with pendant cationic functional groups, are important components in AEMFCs and are responsible for conducting hydroxide anions from the cathode to the anode. Unfortunately, the chemical instability of these cationic polymers towards hydroxide anions remains one of the significant obstacles for the development of viable AEMFCs. Both the polymeric backbones and the tethered cations should be stable to alkaline conditions in order to produce durable devices.

Given that cation degradation under alkaline conditions is a significant problem, many cationic moieties have been investigated for AAEM applications, including ammonium, phosphonium, imidazolium, and metal-based cations. Incorporating piperidinium cations within AAEMs has been suggested due to their alkaline stability. Previously reported piperidinium-functionalized AAEMs are based on a poly(aromatic) backbone. These aromatic structures can make the polymers rigid, and may also be adsorbed by electrocatalysts, thereby reducing the efficiency of electrochemical reactions. Additionally, it has been proposed that the aryl-ether linkages in poly(phenylene oxide)s and polysulfones are unstable under alkaline conditions. More recently, it was demonstrated that poly(aromatic) backbones lacking heteroatoms can undergo oxidative degradation in an operating membrane electrode assembly (MEA). Some aromatic-free AAEMs have been prepared with polyolefin backbones,—Among these materials, PE-based AAEMs have shown promising chemical stability and mechanical integrity. These materials are conveniently prepared from a ring-opening-metathesis polymerization (ROMP)/hydrogenation sequence of cyclooctene (COE) monomers functionalized with various cations, including ammonium, phosphonium, imidazolium, and cobaltocenium. Despite these promising properties of PE-based AAEMs, one significant limitation of these materials is the laborious synthesis of the cationic monomers, which usually requires four to eight linear steps from commercially available 1,5-cyclooctadiene (COD).

### SUMMARY OF THE DISCLOSURE

In an aspect, the present disclosure provides compounds. In various examples, a compound comprises one or more group(s) that can be polymerized in a ring-opening metathesis polymerization (ROMP) or the like and are directly and covalently linked to a quaternary ammonium group, and optionally, comprise one or more additional quaternary ammonium group(s). In various examples, one or more compound(s) are used a monomer(s) in a polymerization reaction (e.g., a polymerization reaction of the present disclosure or the like). In various examples, a compound is made by a method of the present disclosure. In various examples, a compound has the following structure:

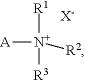

where A is a group that can be polymerized in a ring-opening metathesis polymerization (ROMP) or the like (such as, for example, a cyclooctenyl group, a norbornenyl group, or the like); (i) R¹, R², and R³ are independently at each occurrence an aliphatic group, or (ii) R¹, R², and the ammonium nitrogen (N⁺) taken together form a heterocyclic group where N⁺ is a member of the heterocyclic ring and R³ is an aliphatic group, or (iv) R¹, R², R³, and N taken together form an aliphatic group-bridged heterocyclic group where N⁺ is a member of the heterocyclic ring (e.g., a quinuclidinium group or the like); and X is chosen from halide anions (e.g., F⁻, Cl⁻, Br⁻ or I⁻) and complex anions (such as, for example, BF₄, SbF₆⁻, SbCl₆⁻, PF₆, B(ArF₄)⁻, where ArF₄ is an aryl group substituted with four fluorine groups, B(Ar₄)⁻, where Ar is an aryl group, and the like) and the like. One or more of the aliphatic group(s), if present, a heterocyclic group, or an aliphatic group-bridged heterocyclic group may be covalently bonded (directly or through a linking group) to one or more quaternary ammonium group(s) (e.g.,

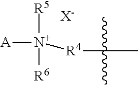

group(s), where R⁴ is an aliphatic group). A

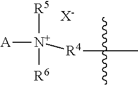

group may be referred to, in the alternative, as a secondary quaternary ammonium. A compound may also comprise one or more linking group(s). In the compound, R¹, R², and N may be together to form a heterocyclic group where at least one of the N atoms is quaternary.

In an aspect, the present disclosure provides methods of making compounds. In various examples, a method comprises a hydroamination reaction using a secondary amine to form a tertiary amine functionalized precursor, and an alkylation reaction or a ring forming oxidation that quaternizes the tertiary amine of the tertiary amine functionalized precursor to form a compound comprising a quaternary ammonium group. A method can be used to make a compound of the present disclosure.

In an aspect, the present disclosure provides polymers. The polymer may be a homopolymer or a copolymer comprising one or more pendant quaternary ammonium(s). In various examples, a polymer does not comprise an aromatic group in the polymer backbone of the polymer. In various examples, a method is made by a method of the present disclosure.

In various examples, a polymer comprises the following structure(s):

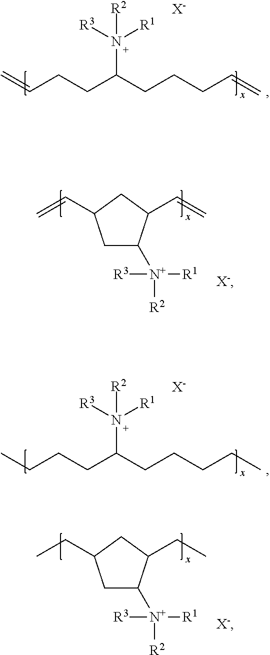

or any combination thereof, where (i) R¹, R², and R³ are independently at each occurrence an aliphatic group, or (ii) R¹, R², and the ammonium nitrogen (N⁺) taken together form a heterocyclic group where N⁺ is a member of the heterocyclic ring and R³ is an aliphatic group, or (iv) R¹, R², R³, and N taken together form an aliphatic group-bridged heterocyclic group where N⁺ is a member of the heterocyclic ring (e.g., a quinuclidinium group or the like); and X is chosen from halides (e.g., —F, —Cl, —Br— or —I) and complex anions (such as, for example, BF₄⁻, SbF₆⁻, PF₆⁻, B(ArF₄)⁻, where ArF₄ is an aryl group substituted with four fluorine groups, B(Ar₄)⁻, where Ar is an aryl group, and the like) and the like; and x is the mol fraction of structure(s) in the polymer and the mol fraction of structures is about 0.01 to about 1. In various examples, the polymer is a homopolymer or a copolymer. In various examples, at least a portion of, substantially all, or all of the carbon-carbon double bonds are hydrogenated.

In an aspect, the present disclosure provides compositions. In various examples a composition comprises one or more polymer(s) of the present disclosure. In various examples, a composition, which may be suitable for making an anion exchange membrane, a liquid (e.g., a solution, a suspension, or the like) comprising one or more polymer(s), and, optionally, further comprises one or more salt(s) (e.g., lithium salt(s), such as, for example, lithium hexafluorophosphate, lithium bis(trifluoromethanesulfonyl)imide (LiTFSI), and the like), or the like, or any combination thereof.

In an aspect, the present disclosure provides methods of making polymers. The methods include a ring-opening metathesis polymerization. In various examples, a polymer is made by a method of the present disclosure.

In various examples, a method of making a polymer comprising the following structure:

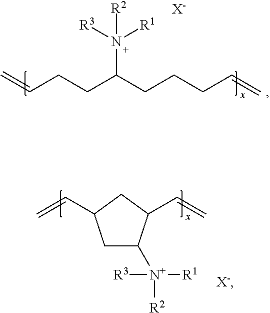

or any combination thereof, where (i) R¹, R², and R³ are independently at each occurrence an aliphatic group, or (ii) R¹, R², and the ammonium nitrogen (N⁺) taken together form a heterocyclic group where N⁺ is a member of the heterocyclic ring and R³ is an aliphatic group, or (iv) R¹, R², R³, and N taken together form an aliphatic group-bridged heterocyclic group where N⁺ is a member of the heterocyclic ring (e.g., a quinuclidinium group or the like); and X is chosen from halides (e.g., —F, —Cl, —Br— or —I) and complex anions (such as, for example, BF₄, SbF₆, PF₆⁻, B(ArF₄)⁻, where ArF₄ is an aryl group substituted with four fluorine groups, B(Ar₄)⁻, where Ar is an aryl group, and the like) and the like; and x is the mol fraction of structure(s) in the polymer and the mol fraction of structures is about 0.01 to about 1, comprises:
polymerizing (in a ring-opening-metathesis polymerization (ROMP)) one or more monomers independently chosen from monomer(s) having the following structure:

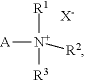

wherein A is a cyclooctenyl group or a norbornenyl group; (i) R¹, R², and R³ are independently at each occurrence an aliphatic group, or (ii) R¹, R², and the ammonium nitrogen (N⁺) taken together form a heterocyclic group where N⁺ is a member of the heterocyclic ring and R³ is an aliphatic group, or (iv) R¹, R², R³, and N taken together form an aliphatic group-bridged heterocyclic group where N⁺ is a member of the heterocyclic ring (e.g., a quinuclidinium group or the like); and X is chosen from halides (e.g., —F, —Cl, —Br— or —I) and complex anions (such as, for example, BF₄, SbF₆⁻, PF₆⁻, B(ArF₄)⁻, where ArF₄ is an aryl group substituted with four fluorine groups, B(Ar₄)⁻, where Ar is an aryl group, and the like) and the like or copolymerizing (i) two or more the monomers or (ii) one of the monomers and one or more additional monomer(s) (monomers other than the ammonium functionalized monomers of the present disclosure), where the polymer is formed. Various additional monomers may be used in a method. A method may comprise hydrogenation of the polymer produced in the method.

In an aspect, the present disclosure provides anion exchange membranes. An anion exchange membrane can be used in device. In various examples, an anion exchange membrane comprises one or more polymer(s) of the present disclosure.

In an aspect, the present disclosure provides uses of polymers of the present disclosure. A polymer can be used in a device as an anion-exchange membrane. A device can comprise one or more anion-exchange membrane(s), each membrane comprising one or more polymer of the present disclosure. In various examples, a device is an energy-storage device, an energy-generating device, or the like. In various examples, an energy device is a battery (e.g., a primary battery, a secondary battery, or the like) or the like. In various examples, a battery is a metal battery, an ion battery, a redox flow battery, or the like. In various examples, an energy-generating device is a fuel cell, or the like. In various examples, a device is a water-electrolysis device or the like. In various examples, in a device, an anion-exchange membrane is a separator and facilitates conduction of or separation of species (depending on the device), such as, for example, ions (which may be cations and/or anions), water, electrolyte(s), and the like, and any combination thereof.

### BRIEF DESCRIPTION OF THE FIGURES

For a fuller understanding of the nature and objects of the disclosure, reference should be made to the following detailed description taken in conjunction with the accompanying figures herein.

FIG. 1 shows a general scheme for preparing functionalized cyclooctene monomers.

FIG. 2 shows a 1,5-cyclooctadiene (COD) hydroamination reaction (see Table 1).

FIGS. 3A-3B show a general schematic for synthesizing (FIG. 3A) piperidinium-and (FIG. 3B) quinuclidinium-functionalized cyclooctenes.

FIG. 4 shows a synthetic scheme for piperidinium-functionalized AAEMs with polyethylene (PE) backbones (PEPs) and quinuclidinium-functionalized AAEMs with PE backbones (PEQs) (see Table 2).

FIGS. 5A-5B show: (FIG. 5A) Membrane hydroxide conductivity and water uptake of methyl-substituted PEPs (PEPMs) with different ionic contents; (FIG. 5B) Membrane hydroxide conductivities of PEPMs under different temperatures.

FIGS. 6A-6B show possible degradation pathways of PEPs.

FIG. 7 shows membrane hydroxide conductivity stability under 1M KOH_{aq} at 80° C. in PP vials for 30 days.

FIG. 8 shows an FT-IR spectra of membranes before and after alkaline treatment to study degradation mechanisms.

FIG. 9 shows stress-strain curves of PEPM₂₀ before and after 1 M KOH_{aq} treatment at 80° C. for 30 days.

FIG. 10 FT-IR spectra of PEPM₂₀, PEPE₂₀, PEPB₁₈, PEPO₂₃, and PEQ₂₀ before and after 1 M KOH_{aq} at 80° C. for 30 days in PP vials. The IR traces for all samples were almost the same.

FIG. 11 shows an FT-IR spectra of PEP₂₀, PEPM₂₀, PEPE₂₀, PEPB₁₈, PEPO₂₃, and PEQ₂₀ before and after 1 M KOH_{aq} at 80° C. for 30 days in soda lime glass vials. Broad peaks near 1050 cm⁻¹ appeared in all samples after the stability study, while the neutral sample PEP20 showed the biggest peak. This peak was attributed to Si—O bonds from glass etching.

FIG. 12 shows a comparison of conductivity changes of PEPM₂₀ in 1 M KOH_{aq} at 80° C. for 30 days in soda lime glass vials (top curve) and in polypropylene (PP) vials (bottom curve). Relatively slower degradation was observed for the samples in glass vials.

FIG. 13 shows a sol-gel transformation PEPM₄₃ ionomer 5 wt % in nPrOH.

FIGS. 14A-14B show alkaline stability evaluation of model compound 6 and oligomers under 2 M KOH/CD₃OH conditions at 80° C. for 30 days: Degradation kinetics by ¹H NMR analysis (FIG. 14A) and byproduct assignment (FIG. 14B).

FIG. 15 shows an experimental setup for 2.0 mmol scale hydroamination reaction.

FIG. 16 shows an experimental setup for 6.0 mmol scale hydroamination reaction.

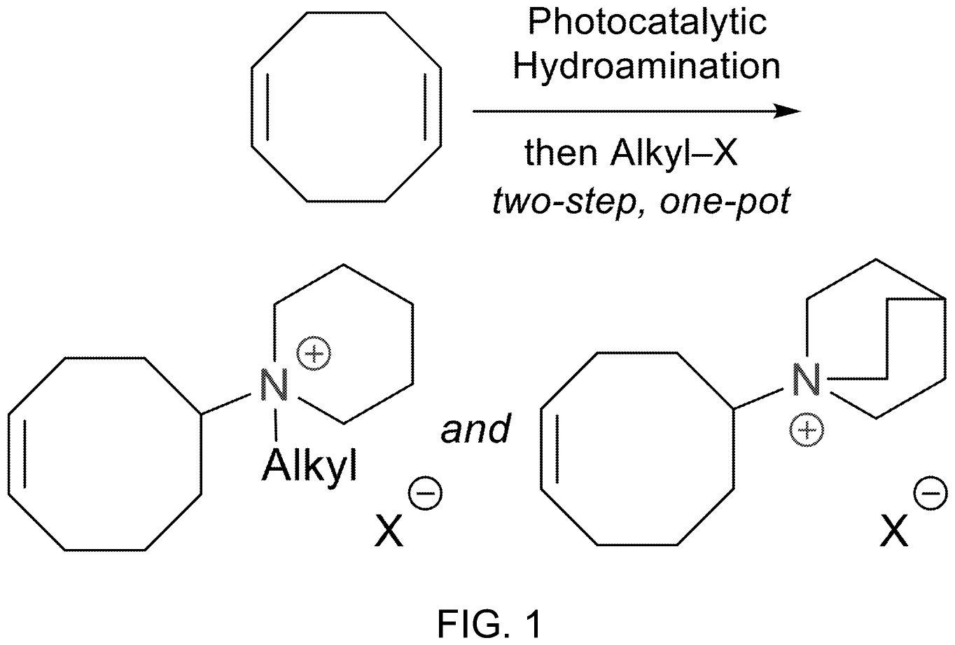

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also, unless otherwise stated, include individual values (e.g., about 1%, about 2%, about 3%, about 4%, etc.) and the sub-ranges (e.g., about 0.5% to about 1.1%, about 0.5% to about 2.4%, about 0.5% to about 3.2%, about 0.5% to about 4.4%, and other possible sub-ranges) within the indicated range. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

As used herein, unless otherwise indicated, “about” or “the like”, when used in connection with a measurable variable (such as, for example, a parameter, an amount, a temporal duration, or the like) or a list of alternatives, is meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations and variations in the alternatives are appropriate to perform in the instant disclosure. As used herein, the terms “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative examples of groups include:

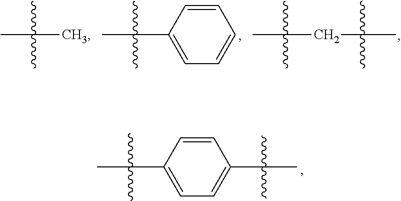

and the like.

As used herein, unless otherwise indicated, the term aliphatic group refers to branched or unbranched hydrocarbon groups that, optionally, contain one or more degree(es) of unsaturation (e.g., groups with different degrees of unsaturation include, but are not limited to, alkenyl groups, alkynyl groups, and cycloaliphatic groups). For example, the aliphatic groups are a C₁ to C₂₀ aliphatic group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). An aliphatic group may be unsubstituted or substituted with one or more substituent(s). Examples of substituents include, but are not limited to, halogens (—F, —Cl, —Br, and —I), aliphatic groups (e.g., alkyl groups, alkenyl groups, alkynyl groups, and the like), halogenated aliphatic groups (e.g., trifluoromethyl group and the like), aryl groups, halogenated aryl groups, alkoxide groups, amine groups, nitro groups, carboxylate groups, carboxylic acids, ether groups, silyl ether groups, alcohol groups, alkyne groups (e.g., acetylenyl groups and the like), cycloaliphatic groups, and the like, and any combination thereof. Groups that are aliphatic include, but are not limited to, alkyl groups, alkenyl groups, alkynyl groups, or carbocyclic groups, and the like.

As used herein, unless otherwise indicated, the term “heteroaliphatic,” refers to aliphatic groups where one or more carbon atoms are independently replaced by one or more atom(s) chosen oxygen, nitrogen, sulfur, and the like, and any combination thereof. In certain examples, one or two carbon atom(s) of an aliphatic group independently replaced by one or more of oxygen atom(s), nitrogen atom(s), sulfur atom(s), or any combination thereof. In various examples, a heteroaliphatic group is a heterocycloaliphatic group or heterocyclic group.

As used herein, unless otherwise indicated, the term “alkyl group” refers to branched or unbranched saturated hydrocarbon groups. Examples of alkyl groups include, but are not limited to, methyl groups, ethyl groups, propyl groups, butyl groups, isopropyl groups, tert-butyl groups, and the like. For example, the alkyl group is C₁ to C₂₀, including all integer numbers of carbons and ranges of numbers of carbons therebetween (e.g., C₁, C₂, C₃, C₄, C₅, C_{6, 20} C₇, C₈, C₉, C₁₀, C₁₁, C₁₂, C₁₃, C₁₄, C₁₅, C₁₆, C₁₇, C₁₈, C₁₉, and C₂₀). The alkyl group may be unsubstituted or substituted with one or more substituent(s). Examples of substituents include, but are not limited to, halogens (—F, —Cl, —Br, and —I), aliphatic groups (e.g., alkyl groups, alkenyl groups, alkynyl groups, and the like), halogenated aliphatic groups (e.g., trifluoromethyl group and the like), aryl groups, halogenated aryl groups, alkoxide groups, amine groups, nitro groups, carboxylate groups, carboxylic acids, ether groups, silyl ether groups, alcohol groups, alkyne groups (e.g., acetylenyl groups and the like), cycloaliphatic groups, and the like, and combinations thereof.

As used herein, unless otherwise indicated, the term “aryl group” refers to C₅ to C₃₀ aromatic or partially aromatic carbocyclic groups, including all integer numbers of carbons and ranges of numbers of carbons therebetween (e.g., C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). In various examples, an aryl group is an aromatic group. Aryl groups include groups such as, for example, fused ring, biaryl groups, or a combination thereof. An aryl group may be unsubstituted or substituted with one or more substituent(s). Examples of substituents include, but are not limited to, halogens (—F, —Cl, —Br, and —I), aliphatic groups (e.g., alkyl groups, alkenyl groups, alkynyl groups, and the like), halogenated aliphatic groups (e.g., trifluoromethyl group and the like), aryl groups, halogenated aryl groups, alkoxide groups, amine groups, nitro groups, carboxylate groups, carboxylic acids, ether groups, silyl ether groups, alcohol groups, alkyne groups (e.g., acetylenyl groups and the like), cycloaliphatic groups, and the like, and any combination thereof. Aryl groups may contain hetero atoms, such as, for example, nitrogen (e.g., pyridinyl groups and the like). Such group may be referred to as heteroaryl groups. Examples of aryl groups include, but are not limited to, phenyl groups, biaryl groups (e.g., biphenyl groups and the like), fused ring groups (e.g., naphthyl groups and the like), hydroxybenzyl groups, tolyl groups, xylyl groups, furanyl groups, benzofuranyl groups, indolyl groups, imidazolyl groups, benzimidazolyl groups, pyridinyl groups, and the like.

As used herein, unless otherwise indicated, the terms “cycloaliphatic”, used alone or as part of a larger moiety, unless otherwise indicated, refer to a saturated or partially unsaturated cyclic aliphatic monocyclic, bicyclic, or polycyclic ring groups, comprising from 3 to 12 atoms, wherein the aliphatic ring(s) are optionally and independently, in the case of bicyclic groups and polycyclic groups substituted as described above. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In some examples, the cycloalkyl group comprises 3 to 6 carbons. The term “cycloaliphatic” also includes aliphatic rings that are fused to one or more aromatic or nonaromatic ring(s), such as, for example, decahydronaphthyl or tetrahydronaphthyl, where the radical or point of attachment is on the aliphatic ring. In various examples, a cycloaliphatic group is spiro ring groups.

The present disclosure provides compounds and methods of making and using same. The present disclosure also provides polymers and methods of making and using same.

In an aspect, the present disclosure provides compounds. In various examples, a compound comprises one or more group(s) that can be polymerized in a ring-opening metathesis polymerization (ROMP) or the like and are directly and covalently linked to a quaternary ammonium group, and optionally, comprise one or more additional quaternary ammonium group(s). In various examples, one or more compound(s) are used a monomer(s) in a polymerization reaction (e.g., a polymerization reaction of the present disclosure or the like). In various examples, a compound is made by a method of the present disclosure. Non-limiting examples of compounds are disclosed herein.

In various examples, a compound has the following structure:

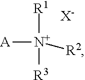

where A is a group that can be polymerized in a ring-opening metathesis polymerization (ROMP) or the like (such as, for example, a cyclooctenyl group, a norbornenyl group, or the like); (i) R¹, R², and R³ are independently at each occurrence an aliphatic group, or (ii) R¹, R², and the ammonium nitrogen (N⁺) taken together form a heterocyclic group where N⁺ is a member of the heterocyclic ring and R³ is an aliphatic group, or (iv) R¹, R², R³, and N taken together form an aliphatic group-bridged heterocyclic group where N⁺ is a member of the heterocyclic ring (e.g., a quinuclidinium group or the like); and X is chosen from halide anions (e.g., F⁻, Cl⁻, Br⁻ or I⁻) and complex anions (such as, for example, BF₄⁻, SbF₆⁻, SbCl₆⁻, PF₆⁻, B(ArF₄)⁻, where ArF₄ is an aryl group substituted with four fluorine groups, B(Ar4)⁻, where Ar is an aryl group, and the like) and the like. One or more of the aliphatic group(s), if present, a heterocyclic group, or an aliphatic group-bridged heterocyclic group may be covalently bonded (directly or through a linking group) to one or more quaternary ammonium group(s) (e.g.,

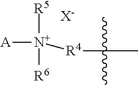

group(s), where R⁴ is an aliphatic group). A

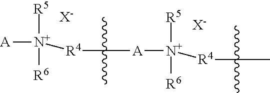

group may be referred to, in the alternative, as a secondary quaternary ammonium. A compound comprising one or more secondary quaternary ammonium group(s) may be referred to, in the alternative, as a functionalized compound. In various examples, A is a ring atom, which may be a carbon atom, of a cycloaliphatic group of a cyclooctenyl group or a norbornenyl group.

In various examples, R¹, R², and N taken together form a heterocyclic group and R³ is an aliphatic group, or R¹, R², R³, and N taken together form an aliphatic group-bridged heterocyclic group (e.g., a quinuclidinium group). In various other example(s) R³ comprises a linking group and a secondary quaternary ammonium, where a first terminus of the linking group is covalently bound to the ammonium nitrogen of the compound and a second terminus of the linking group is covalently bound the secondary quaternary ammonium thru the R⁴ group, and R¹ and R² are independently at each occurrence an aliphatic group or R¹, R², and N taken together form a heterocyclic group. In various other examples R¹ and/or R² each comprises a linking group and a secondary quaternary ammonium where a first terminus of the linking group is covalently bound to the ammonium nitrogen of the compound and a second terminus of the linking group is covalently bound the secondary quaternary ammonium thru the R⁴ group, where R⁴ and R³ are each an aliphatic group. In various other examples, R¹, R², and N taken together form a heterocyclic linking group, where a first terminus of the heterocyclic linking group is covalently bound to the ammonium nitrogen of the compound and a second terminus of the heterocyclic linking group is covalently bound a secondary quaternary ammonium thru the R⁴ group, where R⁴ and R³ are each an aliphatic group. In various other examples, R¹, R², and N taken together form an aliphatic group-bridged heterocyclic linking group (e.g., a quinuclidinium linking group), where a first terminus of the linking bridged heterocyclic group is covalently bound to the ammonium nitrogen of the compound and a second terminus of the aliphatic group-bridged heterocyclic linking group is covalently bound a secondary quaternary ammonium, where R⁴ and R³ are each an aliphatic group; In various examples, R¹, R², R³ are independently, at each occurrence in the compound an aliphatic group (e.g., a C₁ to C₂₀ aliphatic group, including all integer number of carbons therebetween).

A compound may also comprise one or more linking group(s). In various examples, a linking group is an alkyl group, an aryl group, or the like.

In various examples, a compound has the following structure:

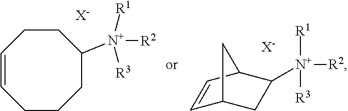

or the like.

In the compound, R¹, R², and N may be together to form a heterocyclic group where at least one of the N atoms is quaternary. Non-limiting examples of such heterocyclic groups include piperidinyl groups (such as, for example, (4-amino)piperidinyl groups, (4-ol)piperidinyl groups), and the like), pyrrolidinyl groups, azepanyl groups, morpholinyl groups, piperazinyl groups, 1,4-Diazabicyclo[2.2.2]octane (DABCO) groups, analogs or derivatives thereof (e.g., as described herein), and the like.

In various examples, a compound comprises a heterocyclic group where at least one of the N atoms in the heterocyclic ring of the heterocyclic group is quaternary. In various examples, the compound has the following structure:

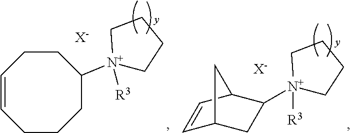

where y is 1, 2, 3, or 4, analogs or derivatives thereof (e.g., as described herein), or the like.

In various examples, a compound comprises a piperidinyl group where at least one of the N atoms is quaternary (e.g., a piperidinium group). In various examples, the compound has the following structure:

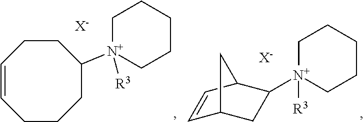

analogs or derivatives thereof (e.g., as described herein), or the like.

In various examples, R¹, R², and N taken together form an aliphatic group-bridged piperidinium group (i.e., a quinuclidinium group) and the compound has the following structure:

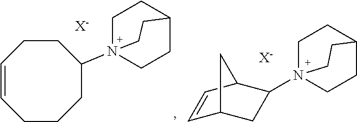

or the like, analogs or derivatives thereof (e.g., as described herein), or the like.

In various examples, R¹, R², and N taken together form an aliphatic group-bridged heterocyclic group. In various examples, the compound has the following structure:

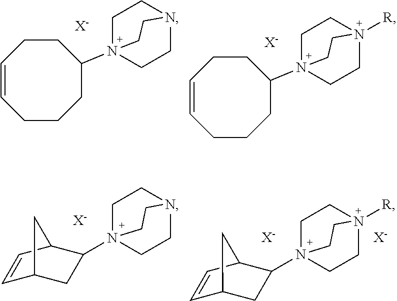

where R is an aliphatic group or an aryl group, analogs or derivatives thereof (e.g., as described herein), or the like.

A compound may comprise at least two quaternary ammonium groups (e.g., a quaternary ammonium directly and covalently linked to a group that can be polymerized in a ring-opening metathesis polymerization (ROMP) or the like (such as, for example, a cyclooctenyl group, a norbornenyl group, or the like) and one or more additional (e.g., secondary) quaternary ammonium group(s). These compounds may be referred to, in the alternative, as multifunctional compounds. In various examples, a compound comprises two quaternary ammonium groups linked directly to each other via an aliphatic group, a heterocyclic group where at least one of the N atoms is quaternary, or a bridged heterocyclic group where at least one of the N atoms is quaternary of a first quaternary ammonium group and an aliphatic group, a heterocyclic group where at least one of the N atoms is quaternary, or a bridged heterocyclic group where at least one of the N atoms is quaternary of a second quaternary group. In various examples, a compound comprises two quaternary ammonium groups linked directly to each other via a linking group. Non-limiting examples of linking groups include aliphatic groups, aryl groups, heterocyclic groups, and the like. In various examples, a compound comprising at least two quaternary ammonium groups has the following structure:

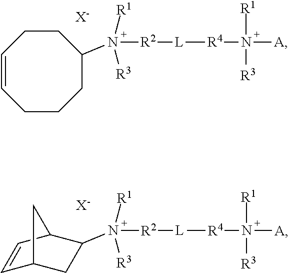

where L is an optional linking group and R⁴ is an aliphatic group, analogs or derivatives thereof (e.g., as described herein), or the like.

In various examples, a multifunctional compound, which may comprise a linking group, has the following structure.

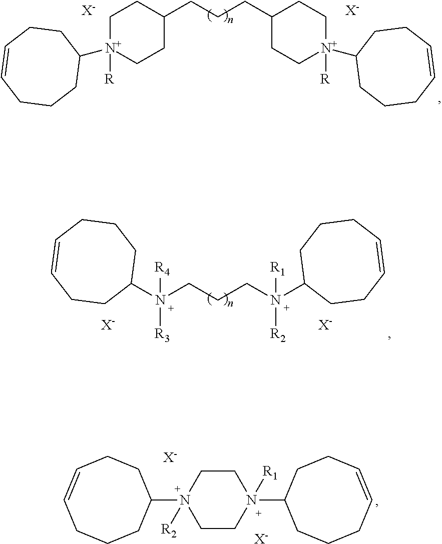

and the like, and norbornenyl derivatives thereof.

In certain examples, the compound (e.g., the quaternary ammonium-functionalized cyclooctene group) does not further comprise a spacer group between the compound (e.g., the cyclooctene group) and the quaternary ammonium-functionalized group (e.g., the quaternary nitrogen atom of the quaternary ammonium group).

In an aspect, the present disclosure provides methods of making compounds. In various examples, a method comprises a hydroamination reaction using a secondary amine to form a tertiary amine functionalized precursor, and an alkylation reaction or a ring forming oxidation that quaternizes the tertiary amine of the tertiary amine functionalized precursor to form a compound comprising a quaternary ammonium group. A method can be used to make a compound of the present disclosure. Non-limiting examples of methods of making are described herein.

In various examples, a method of making a compound of the present disclosure comprises: forming a first reaction mixture comprising: 1,5 cyclooctadiene, norbornadiene, or any combination thereof; one or more secondary amine(s), one or more hydroxyalkyl secondary amine(s), one or more multifunctional secondary amine(s), or any combination thereof one or more H-atom transfer (HAT) catalyst(s); and one or more photocatalyst(s); subjecting the first reaction mixture to electromagnetic radiation to form one or more precursor compound(s) (which may be tertiary amine-functionalized precursor compound(s), such as, for example, the tertiary amine-functionalized cyclooctene compound(s) and/or the tertiary amine-functionalized norbornene compound(s)); forming a second reaction mixture comprising: the tertiary amine-functionalized cyclooctene compound(s) and/or the tertiary amine-functionalized norbornene compound(s), or any combination thereof; and one or more alkylating agent(s) (e.g., hydrocarbon halide(s), one or more trialkyloxonium salt(s)), or a combination thereof; or the hydroxyalkyl amine-functionalized cyclooctene compound(s), the hydroxyalkylamine-functionalized norbornene compound(s), functionalized tertiary amine-functionalized norbornadiene compound(s), or any combination thereof, one or more reductant(s); and one or more bromine source(s), where the second reaction mixture forms one or more of the compound(s) (e.g., one or more quaternary ammonium-functionalized cyclooctene compound(s), one or more quaternary ammonium-functionalized norbornene compound(s), one or more functionalized quaternary ammonium-functionalized cyclooctene compound(s), one or more functionalized quaternary ammonium-functionalized norbornene compound(s), or any combination thereof).

A method may comprise various additional processes. In various examples, a method further comprises one or more or all of the following: isolating the tertiary amine-functionalized cyclooctene compound(s), the tertiary amine-functionalized norbornadiene compound(s), the functionalized tertiary amine-functionalized cyclooctene compound(s), the functionalized tertiary amine-functionalized norbornadiene compound(s) or any combination thereof), prior to formation of the second reaction mixture; or isolating the tertiary amine-functionalized cyclooctene compound or the (bridged piperidinium)-functionalized cyclooctene compound from the second reaction mixture. Suitable isolation methods are known in the art. In various examples, precursor compound (e.g., tertiary amine-functionalized precursor compound(s), such as, for example, the tertiary amine-functionalized cyclooctene compound(s) and/or the tertiary amine-functionalized norbornene compound(s)) is/are isolated by chromatographic method(s), distillation, or the like. In various examples, compound(s) (e.g., one or more quaternary ammonium-functionalized cyclooctene compound(s), one or more quaternary ammonium-functionalized norbornene compound(s), one or more functionalized quaternary ammonium-functionalized cyclooctene compound(s), one or more functionalized quaternary ammonium-functionalized norbornene compound(s), or any combination thereof) is/are isolated by precipitation(s) and filtration(s), or the like.

A first reaction mixture can comprise various amounts of secondary amine(s). In various examples, the 1,5-cyclooctadiene and/or norbornadiene is present in the first reaction mixture at about 0.25 molar equivalents to about 50 molar equivalents, based on the total moles of secondary amine(s), one or more hydroxyalkyl amine(s), or any combination thereof, including all 0.01 M values and ranges therebetween.

Various secondary amines, hydroxyalkyl secondary amines, multifunctional secondary amines, or any combination thereof, can be used in a method. Suitable examples of secondary amines, hydroxyalkyl secondary amines, and multifunctional secondary amines can made by methods known in the art and are commercially available.

In various examples, a secondary amine comprises two aliphatic groups, an aliphatic group and carbocyclic group, or two carbocyclic groups. In various examples, a secondary amine is a secondary alkyl amine, N-alkyl-N-aryl amines, or the like, or any combination thereof. Non-limiting examples of secondary amines include piperidines, pyrrolidines, azepanes, morpholines, piperazines, piperidin-4-amine, piperidin-4-ol, 1,4-Diazabicyclo[2.2.2]octane (DABCO), N-methylcyclohexanamine, 2,2-imethoxy-N-methylethan-1-amine, N-methyl-2-(pyridine-4-yl)ethan-1-amine, and the like and derivatives and analogs thereof (e.g., as described herein). Other non-limiting examples of secondary amines include:
N(H)R′R″, wherein R′ and R″ are independently chosen from aliphatic groups (such as, for example,

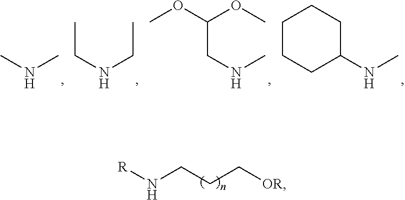

the R N-substituent is an aliphatic group, the R O-substituent is H or an aliphatic group,

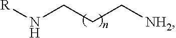

where R⁹ is an aliphatic group,

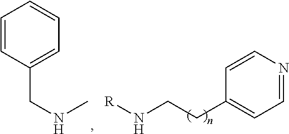

such as for example,

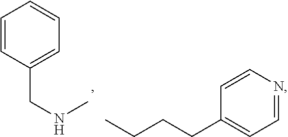

and the like) or where R′ and R″ taken together form a cyclic aliphatic group, which may comprise one or more heteroatom(s), (such as, for example,

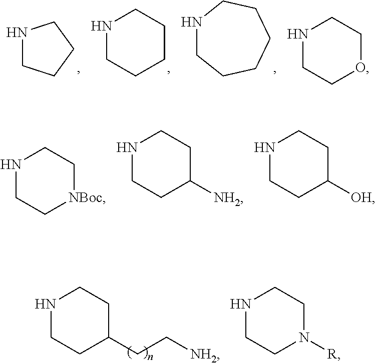

where R is an aliphatic group or aryl group, where R is an —NH₂ group or an —OH group,

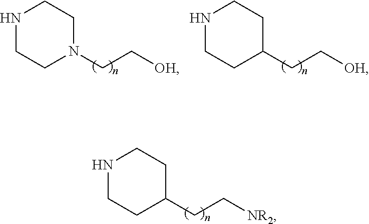

wherein the R groups are both —H groups or both aliphatic groups,

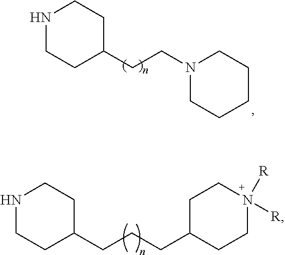

where R is independently at each occurrence an aliphatic group,

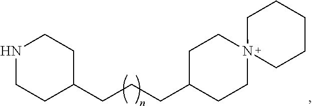

or the like, where in each case n is 0 to 6 (e.g., 0, 1, 2, 3, 4, 5, or 6).

Non-limiting examples of hydroxyalkyl secondary amine(s)s include:

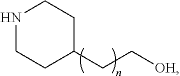

where n is 0 to 6 (e.g., 0, 1, 2, 3, 4, 5, or 6) (such as, for example, 2-(piperidin-4-yl)ethanol, and the like, and any combination thereof), and the like, and any combination thereof.

Non-limiting examples of multifunctional secondary amines include:

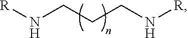

where R independently, at each occurrence, is an aliphatic group,

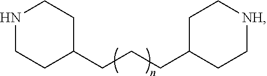

where in each case n is 0 to 6 (e.g., 0, 1, 2, 3, 4, 5, or 6).

A first reaction mixture can comprise various amounts of secondary amine(s), hydroxyalkyl amine(s), multifunctional secondary amine(s), and any combination thereof. In various examples, the first reaction mixture comprises the secondary amine(s), hydroxyalkyl amine(s), multifunctional secondary amine(s), or any combination thereof, at a concentration (in the aggregate) of from about 0.01 M to about 5 M (based on the total volume of the reaction mixture), including all 0.005 M values and ranges therebetween.

Various H-atom transfer (HAT) catalysts can be used in a method. Without intending to be bound by any particular theory, it is considered that a HAT catalyst transfers a hydrogen during the reaction of 1,5 cyclooctadiene, norbornadiene, or any combination thereof with the secondary amine(s), hydroxyalkyl secondary amine(s), multifunctional secondary amine(s), or any combination thereof. Suitable examples of H-atom transfer (HAT) catalyst can made by methods known in the art and are commercially available. Non-limiting examples of H-atom transfer (HAT) catalysts include thiophenol, substituted thiophenols, phenol, substituted phenols, thiols, malonitrile, hydroxamic acid, silanes, and the like, and any combinations thereof.

A first reaction mixture can comprise various amounts of H-atom transfer (HAT) catalyst(s). In various examples, the first reaction mixture comprises the H-atom transfer (HAT) catalyst(s) at a concentration (in the aggregate) of about 0.1 mol % to about 500 mol %, based on the total moles of secondary amine(s), hydroxyalkyl amine(s), multifunctional secondary amine(s), or any combination thereof, including all 0.1 mol % values and ranges therebetween.

Various photocatalyst catalysts can be used in a method. Suitable examples of photocatalysts can made by methods known in the art and are commercially available. Non-limiting examples photocatalyst catalysts include iridium-bipyridine photocatalysts, acridinium photocatalysts, and the like, and any combination thereof.

In various examples, an iridium-bipyridine photocatalyst has or iridium photocatalyst(s) have the following structure:

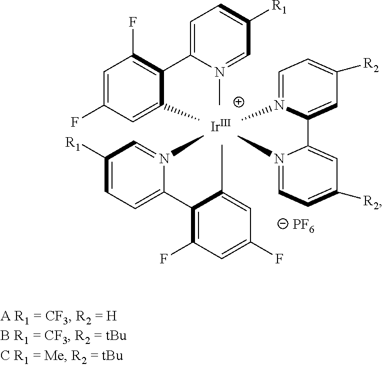

or the like, or any combination thereof. In various examples, an iridium-bipyridine photocatalyst is Iridium(III) bis[2-(2,4-difluorophenyl)-5-methylpyridine-N,C20]-4,40-di-tert-butyl-2,20-bipyridine hexafluorophosphate ([Ir(dF(Me)ppy)₂(dtbbpy)]PF₆) or the like.

In various examples, an acridinium photocatalyst has or acridionium photocatalyst(s) have the following structure:

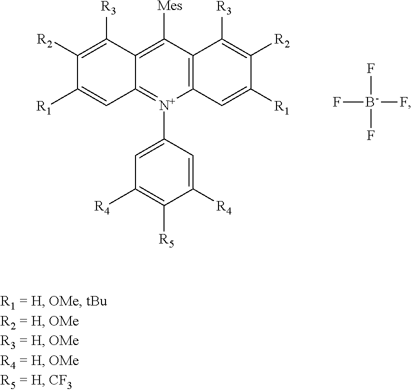

or the like. In various examples, an acridinium photocatalyst is 9-mesityl-3,6-di-tert-butyl-10-phenylacridinium tetrafluoroborate, or the like, or any combination thereof.

A first reaction mixture can comprise various amounts of photocatalyst(s). In various examples, the first reaction mixtures comprises the photocatalyst photocatalyst(s) at a concentration (in the aggregate) of about 0.01 mol % to about 10 mol %, based on the total moles of pyridine, one or more hydroxyalkyl pyridine(s), or any combination thereof, including all 0.1 mol % values and ranges therebetween.

A first reaction mixture may comprise various solvent(s). Non-limiting examples of solvents include non-polar solvents, cyclic ethers (such as, for example, tetrahydrofuran, dioxane and the like), aromatic solvents (such as, for example, benzene, toluene, xylene, and the like), aprotic solvents (such as, for example, acetonitrile, and the like), and the like, and any combination thereof. A second reaction mixture may comprise various solvent(s). Non-limiting examples of solvents include cyclic ethers (such as, for example, tetrahydrofuran and the like), chlorinated solvents (such as, for example, dichloromethane, and the like), aprotic solvents (such as, for example, acetonitrile, and the like), and the like, and any combination thereof.

In various examples, the electromagnetic radiation initiate(s) a hydroamination reaction (which may form a precursor compound). The electromagnetic radiation can be provided by various sources. In various examples, the electromagnetic radiation is provided a source, such as, for example, a laser or broad band source (e.g., a light emitting diode (such as, for example, a blue LED or the like), or the like. In various examples, the electromagnetic radiation comprises one or more wavelength(s) that initiate(s) a hydroamination reaction. In various examples, the electromagnetic radiation comprises one or more wavelength(s) from about 350 nm to about 700 nm (e.g., electromagnetic radiation comprising wavelengths from about 450 nm to about 475 nm, including all 0.1 nm values and ranges therebetween).

The first reaction mixture can be subjected to the electromagnetic radiation for various durations. In various examples, the first reaction mixture is subjected to the electromagnetic radiation for about 5 minutes to about 168 hours, including all 0.1 hour values and ranges therebetween. The first reaction mixture can be subjected to the electromagnetic radiation under various conditions. In various examples, the first reaction mixture is subjected to the electromagnetic radiation at a temperature of about −50° C. to about 110° C., including all 0.1° C. values and ranges therebetween. In various examples, the first reaction mixture under an inert (e.g., nitrogen, argon, or the like) or ambient atmosphere (e.g., air at about 1 atm (depending on altitude).

A precursor compound comprises a cyclooctene group or norbornene group and at least one tertiary amine group (which is formed from the secondary amine and is covalently and directly bonded to the cyclooctene group or norbornene group). The precursor compound can be subjected to one or more additional reaction(s) that quaternize the nitrogen atom of the tertiary amine group. In various examples, a precursor compound is subjected to an alkylation reaction or a ring-forming oxidation reaction, or the like. Non-limiting examples of precursor compounds include (e.g., tertiary amine-functionalized cyclooctene compound(s), one or more tertiary amine-functionalized norbornadiene compound(s), one or more functionalized tertiary amine-functionalized cyclooctene compound(s), one or more functionalized tertiary amine-functionalized norbornadiene compound(s), one or more hydroxyalkyl amine-functionalized cyclooctene compound(s), one or more hydroxyalkylamine-functionalized norbornadiene compound(s), or any combination thereof).

In the case where the quaternary ammonium-functionalized cyclooctene compound(s), the quaternary ammonium-functionalized norbornadiene compound(s), the functionalized quaternary ammonium-functionalized cyclooctene compound(s), the functionalized quaternary ammonium-functionalized norbornadiene compound(s), or any combination thereof are produced, second reaction mixture comprises the tertiary amine-functionalized cyclooctene compound(s), the tertiary amine-functionalized norbornadiene compound(s), the functionalized tertiary amine-functionalized cyclooctene compound(s), the one or more functionalized tertiary amine-functionalized norbornadiene compound(s), or any combination thereof and the one or alkylating agent(s).

Various alkylating agents and amounts of alkylating agents can be used in a method. Non-limiting examples of alkylating agents include hydrocarbon halides, trialkyl oxonium salts, and the like. In various examples, the alkylating agent(s) is/are one or more hydrocarbon halide(s) chosen from C₁-C₂₀ alkyl halides, trialkyloxonium salts, wherein the alkyl groups are independently chosen from C₁-C₂₀ alkyl groups (e.g., where the anion is a complex anion, such as, for example, BF₄⁻, SbF₆⁻, SbCl₆⁻, PF₆⁻, or the like), and any combination thereof, and wherein the halide group is chosen from iodide (—I), bromide (—Br), chloride (—Cl), and the like, and any combination thereof. In various examples, the alkylating agent is added at from about 0.1 to about 50 molar equivalents based on the total moles of the precursor compound(s) (e.g., tertiary amine-functionalized precursor compound(s), such as, for example, the tertiary amine-functionalized cyclooctene compound(s) and/or the tertiary amine-functionalized norbornene compound(s), or the like, or any combination thereof).

In the case where the (bridged heterocyclic group)-functionalized cyclooctene compound(s), the (bridged heterocyclic group)-functionalized norbornadiene compound(s), or any combination thereof are produced, when the second reaction mixture comprises the hydroxyalkyl amine-functionalized cyclooctene compound(s), the hydroxyalkylamine-functionalized norbornadiene compound(s), or any combination thereof, the reductant(s), and bromine source(s).

Various reductants can be used in a method. In various examples, a reductant is a phosphine reductant or the like. Non-limiting examples of phosphine reductants include triarylphosphines (such as, for example, triphenyl phosphine), trialkylphosphines, substituted derivatives and analogs thereof, and the like.

Various bromine sources can be used in a method. Non-limiting examples of phosphine reductants include CBr₄, Br₂, N-bromosuccinimide (NBS), and the like, and any combination thereof.

A second reaction mixture can be reacted under various conditions. In various examples, a second reaction mixture is heated to, cooled to, or held, or a combination thereof at temperature of from about −50° C. to about 120° C., including all 0.1° C. values and ranges therebetween, when the second reaction mixture tertiary amine-functionalized cyclooctene compound(s), the tertiary amine-functionalized norbornadiene compound(s), the functionalized tertiary amine-functionalized cyclooctene compound(s), the one or more functionalized tertiary amine-functionalized norbornadiene compound(s), or any combination thereof and the alkylating agent(s). In various examples, a second reaction mixture is heated to, cooled to, or held, or a combination thereof to a temperature of about −20° C. to about 80° C., including all 0.1° C. values and ranges therebetween, when the second reaction mixture comprises the one or more hydroxyalkyl amine-functionalized cyclooctene compound(s), one or more hydroxyalkylamine-functionalized norbornadiene compound(s), or any combination thereof, the reductant(s), and bromine source(s). In various examples, a first reaction mixture and/or a second reaction mixture is reacted (e.g., held at a desired temperature) for about 1 minute to about seven days (e.g., about 4 hours to about 24 hours), including all 0.1 minute values and ranges therebetween.

In an aspect, the present disclosure provides polymers. The polymer may be a homopolymer or a copolymer comprising one or more pendant quaternary ammonium(s). In various examples, a method is made by a method of the present disclosure. Non-limiting examples of polymers are described herein.

In various examples, a polymer comprises the following structure(s) (repeat unit(s)):

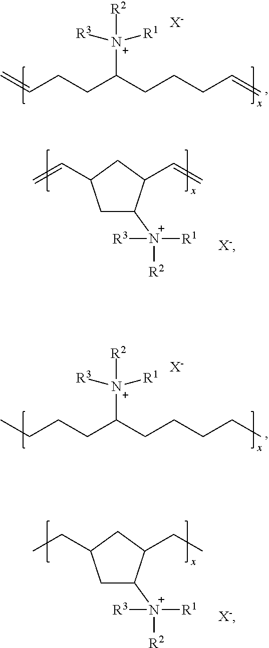

or any combination thereof, where (i) R¹, R², and R³ are independently at each occurrence an aliphatic group, or (ii) R¹, R², and the ammonium nitrogen (N⁺) taken together form a heterocyclic group where N⁺ is a member of the heterocyclic ring and R³ is an aliphatic group, or (iv) R¹, R², R³, and N taken together form an aliphatic group-bridged heterocyclic group where N⁺ is a member of the heterocyclic ring (e.g., a quinuclidinium group or the like); and X is chosen from halides (e.g., —F, —Cl, —Br— or —I), complex anions (such as, for example, BF₄⁻, SbF₆⁻, PF₆⁻, B(ArF₄)⁻, where ArF₄ is an aryl group substituted with four fluorine groups, B(Ar₄)⁻, where Ar is an aryl group, and the like), and the like, and any combination thereof; and x is the mol fraction of structure(s) in the polymer and the mol fraction of structure(s) (repeat unit(s)) is/are about 0.01 to about 1, including all 0.005 mol fraction values and ranges therebetween. In various examples, the polymer is a homopolymer or a copolymer. In various examples, at least a portion of, substantially all, or all of the carbon-carbon double bonds are hydrogenated.

It can be desirable a polymer does not comprise any aromatic groups in the polymer backbone of the polymer. In various examples, a polymer does not comprise an aromatic group in the polymer backbone of the polymer.

A polymer can be a copolymer. In various examples, a copolymer is a block copolymer a, a random copolymer, a tapered copolymer, or the like. In various examples, a copolymer comprises two or more repeat unit(s) (repeat units formed from a monomer of the present disclosure). In various examples, a copolymer comprises one or more repeat unit(s) (repeat units formed from a monomer of the present disclosure) and one or more additional repeat unit(s) (additional repeat units are repeat units other than those formed from a monomer of the present disclosure).

A copolymer can comprise various additional repeat units. Additional repeat unit(s) may be present in a polymer as one or more block(s), randomly distributed in the polymeror the like. In various examples, an additional repeat unit or additional repeat units is/are formed from monomer(s) that can be polymerized in ring-opening metathesis polymerizations and the like. In various examples, a polymer is formed by copolymerization with 1,5-cyclooctadiene, norbornadiene, or the like. In various examples, an additional repeat unit or additional repeat units is/are are chosen from hydrocarbon repeat units, and the like. In various examples, one or more or all of the repeat unit(s) comprises (or is) an aliphatic group.

In certain examples, the polymer does not comprise a spacing group, alternatively referred to as a spacer, between the polymer backbone and one or more quaternary ammonium group(s), functionalized quaternary ammonium group(s), or the like, or any combination thereof, pendant from the polymer backbone. In certain examples, the polymer backbone does not further comprise one or more functionalized quaternary ammonium group(s) pendant from the polymer backbone. In certain examples, the polymer backbone does not further comprise one or more non-quaternary ammonium cationic group(s) pendant from the polymer backbone. In certain examples, the polymer does not comprise one or more cyclic rings, aromatic rings, or the like, or any combination thereof, within the polymer backbone. In certain examples, the polymer does not comprise one or more piperidinium ring carbons, (bridged-pyridinium) ring carbons, or the like, or any combination thereof, within the polymer backbone. In certain examples, the polymer backbone is not directly and covalently bound via a ring carbon atom to one or more piperidinium group(s), one or more (bridged-pyridinium) group(s), or the like, or any combination thereof.

A polymer can have various molecular weights. In various examples, a polymer has a molecular weight (Mw and/or Mn) of 500 g/mol to 1,000,000 g/mol (e.g., 5,000 g/mol to 250,000 g/mol), including all 0.1 g/mol values and ranges therebetween. Methods of determining molecular mass are known in the art. Non-limiting examples of determining molecular mass include gel permeation chromatography (GPC) and the like. In various examples, molecular weight (Mw and/or Mn) is determined by GPC.

A polymer can have various end groups. The end groups can be those resulting from the polymerization reaction(s) or the end groups can result from post polymerization reaction(s) chosen to provide desired end groups. Suitable post polymerization reactions are known in the art. In various examples, at least a portion of or all of the terminal carbons is/are, independently at each occurrence, substituted with an aryl group, hydrogen group, an alkyl group, a halogen, a hydroxyl group, and/or at least a portion of the terminal carbons is, independently at each occurrence, a carbonyl carbon of an aldehyde group, a ketone group, an acid group, an acetate group, or the like. Methods of chain end analysis are known in the art. Non-limiting examples of chain end analysis include ¹H and ¹³C NMR, mass spectrometry, IR spectroscopy, Raman spectrometry, and the like). In various examples, the chain end(s) of a polymer are determined by one or more of these chain end analyses.

In various example(s), the polymer comprises the following structure:

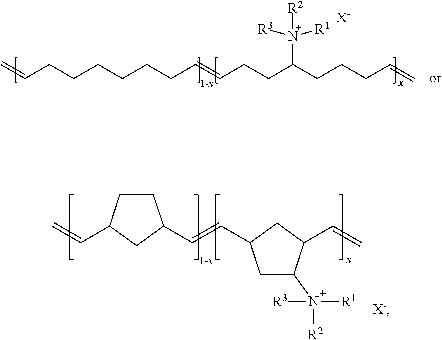

or the like.

In various example(s), the polymer comprises the following structure:

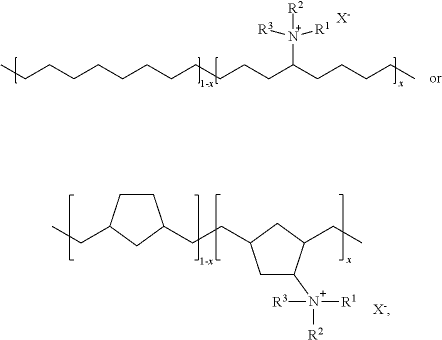

or the like.

A polymer can have various forms. In various examples, a polymer is in the form of a solution, an emulsion, a slurry, a dispersion, a melt, a particle, a flake, a pellet, a powder, a granule, a tube, a sphere, a fiber, a foam, a film, a textile, a mesh, a sheet, a bar, a monolith, or the like.

A polymer can be in the form of a film. A film can have various sizes, dimensions, etc. In various examples, a film has an area of about 1 cm² to about 500 cm², including all 0.1 cm² values and ranges therebetween. In various examples, a film a thickness (e.g., a dimension substantially perpendicular or perpendicular to a longest linear dimension or largest surface of the film or the like) of 1 micron to 500 microns (e.g., 20 microns to 100 microns), including all 0.1 micron values and ranges therebetween.

In an aspect, the present disclosure provides compositions. In various examples a composition comprises one or more polymer(s) of the present disclosure.

A composition can be suitable for use to make an anion exchange membrane of the present disclosure. In various examples, a composition, which may a liquid (e.g., a solution, a suspension, or the like) comprises one or more polymer(s), and, optionally, further comprises one or more salt(s) (e.g., lithium salt(s), such as, for example, lithium hexafluorophosphate, lithium bis(trifluoromethanesulfonyl)imide (LiTFSI), and the like), or the like, or any combination thereof.

In an aspect, the present disclosure provides methods of making polymers. The methods include a ring-opening metathesis polymerization. In various examples, a polymer is made by a method of the present disclosure. Non-limiting examples of methods of making polymers are described herein.

In various examples, a method of making a polymer comprising the following structure:

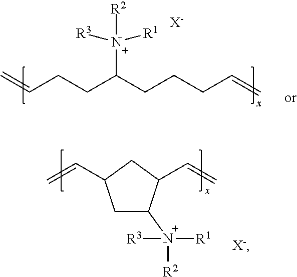

or any combination thereof, where (i) R¹, R², and R³ are independently at each occurrence an aliphatic group, or (ii) R¹, R², and the ammonium nitrogen (N⁺) taken together form a heterocyclic group where N⁺ is a member of the heterocyclic ring and R³ is an aliphatic group, or (iv) R¹, R², R³, and N taken together form an aliphatic group-bridged heterocyclic group where N⁺ is a member of the heterocyclic ring (e.g., a quinuclidinium group or the like); and X is chosen from halides (e.g., —F, —Cl, —Br— or —I) and complex anions (such as, for example, BF₄⁻, SbF₆⁻, PF₆⁻, B(ArF₄)⁻, where ArF₄ is an aryl group substituted with four fluorine groups, B(Ar₄)⁻, where Ar is an aryl group, and the like) and the like; and x is the mol fraction of structure(s) in the polymer and the mol fraction of structures is about 0.01 to about 1, comprises: polymerizing (in a ring-opening-metathesis polymerization (ROMP)) one or more monomers independently chosen from monomer(s) having the following structure:

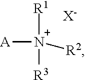

wherein A is a cyclooctenyl group or a norbornenyl group; (i) R¹, R², and R³ are independently at each occurrence an aliphatic group, or (ii) R¹, R², and the ammonium nitrogen (N⁺) taken together form a heterocyclic group where N⁺ is a member of the heterocyclic ring and R³ is an aliphatic group, or (iv) R¹, R², R³, and N taken together form an aliphatic group-bridged heterocyclic group where N⁺ is a member of the heterocyclic ring (e.g., a quinuclidinium group or the like); and X is chosen from halides (e.g., —F, —Cl, —Br— or —I) and complex anions (such as, for example, BF₄⁻, SbF₆⁻, PF₆⁻, B(ArF₄)⁻, where ArF₄ is an aryl group substituted with four fluorine groups, B(Ar₄)⁻, where Ar is an aryl group, and the like) and the like, or copolymerizing (i) two or more the monomers or (ii) one of the monomers and one or more additional monomer(s) (monomers other than the ammonium functionalized monomers of the present disclosure) (co-monomer(s)), where the polymer is formed.

In certain examples, one or more of the pyridinium-functionalized cyclooctene monomer(s), the (bridged-pyridinium)-functionalized cyclooctene monomer(s), or the like, or any combination thereof, do not further comprise a spacer group between the cyclooctene group and the functionalized group. In certain examples, the polymer backbone does not comprise one or more cyclic or aromatic rings. In certain examples, the polymer backbone does not comprise one or more piperidinium ring carbons, (bridged-pyridinium) ring carbons, or the like, or any combination thereof. In certain examples, the piperidinium group(s), the (bridged-pyridinium) group(s), or the like, or any combination thereof, are not pendant from the polymer backbone via a ring carbon.

Various additional can monomers used in a method. Combinations of additional monomers can be used. In various examples, an additional monomer is chosen from unsaturated cyclic hydrocarbon monomers (such, as, for example, cyclooctadiene, norbornadiene, cis-cyclooctene, trans-cyclooctene, norbornene, cyclopentene, cyclobutene, and the like, and any combination thereof), and the like, and any combination thereof. In certain examples, the additional monomers (e.g., the unsaturated cyclic hydrocarbon monomers) do not comprise additional cationic groups (cationic groups other than the ammonium groups of the present disclosure). In certain examples, a method does not comprise ring-opening metathesis polymerization of the monomer(s), with cis-cyclooctadiene and one or more additional cyclooctene monomer(s) functionalized with the pendent cationic group(s).

A method may comprise hydrogenation of the polymer produced in the method. In various examples, a method further comprises hydrogenating the polymer produced by the ROMP. The hydrogenating can be carried out using methods known in the art. In various examples, the hydrogenating is carried out by hydrogenation with, for example, hydrogen gas or the like (which may be carried out in the presence of a catalyst, or the like. Suitable hydrogenation agents and catalysts are known in the art.

In an aspect, the present disclosure provides anion exchange membranes. An anion exchange membrane can be used in device. In various examples, an anion exchange membrane comprises one or more polymer(s) of the present disclosure. Non-limiting examples of anion exchange membranes are descried herein.

An anion exchange membrane can have various thicknesses. In various examples, an anion exchange membrane has a thickness (e.g., a dimension substantially perpendicular or perpendicular to a longest linear dimension or largest surface of the anion exchange membrane or the like) of 1 micron to 500 microns (e.g., 20 microns to 100 microns), including all 0.1 micron values and ranges therebetween.

An anion exchange membrane can be made by methods known in the art. In various examples, an anion exchange membrane is made by solvent casting, melt pressing, reactive casting, or the like.

In an aspect, the present disclosure provides uses of polymers of the present disclosure. A polymer can be used in a device as an anion-exchange membrane. Non-limiting examples of devices are described herein.

A device can comprise one or more anion-exchange membrane(s), each membrane comprising one or more polymer of the present disclosure. In various examples, a device is an energy-storage device, an energy-generating device, or the like. In various examples, a device is an energy-storage device, an electrochemical device. In various examples, an energy device is a battery (e.g., a primary battery, a secondary battery, or the like) or the like. In various examples, a battery is a metal battery, an ion battery, a redox flow battery, or the like. In various examples, an energy-generating device is a fuel cell, or the like. In various examples, a device is a water-electrolysis device or the like. In various examples, in a device, an anion-exchange membrane is a separator and facilitates conduction of or separation of species (depending on the device), such as, for example, ions (which may be cations and/or anions), water, electrolyte(s), and the like, and any combination thereof.

The following Statements describe various examples of methods, products and systems of the present disclosure and are not intended to be in any way limiting:
Statement 1. A compound comprising (or having) the following structure:

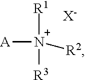

where A is a cyclooctenyl group or a norbornenyl group; R¹, R², and R³ are independently at each occurrence an aliphatic group, or R¹, R², and N taken together form a heterocyclic group and R³ is an aliphatic group, or R¹, R², R³, and N taken together form an aliphatic group-bridged heterocyclic group (e.g., a quinuclidinium group), or R³ comprises a linking group and a

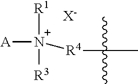

group, where a first terminus of the linking group is covalently bound to the quaternary ammonium nitrogen of the compound and a second terminus of the linking group is covalently bound the

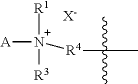

group through the R⁴ group, and R¹ and R² are independently at each occurrence an aliphatic group or R¹, R², and the N of the compound taken together form a heterocyclic group and R⁵ and R⁶ are independently at each occurrence an aliphatic group or R⁵, R⁶, and the

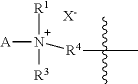

group taken together form a heterocyclic group, or R¹ and/or R² each comprises a linking group and a

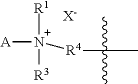

group, where a first terminus of the linking group is covalently bound to the quaternary ammonium nitrogen of the compound and a second terminus of the linking group is covalently bound the

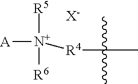

group through the R⁴ group, where the R⁴ group is an aliphatic group, R³ is an aliphatic group, and R⁵ and R⁶ are independently at each occurrence an aliphatic group or R⁵, R⁶, and the N of the

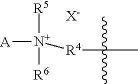

group taken together form a heterocyclic group, or R¹, R², and N taken together form a heterocyclic linking group, where a first terminus of the heterocyclic linking group is covalently bound to the quaternary ammonium nitrogen of the compound and a second terminus of the heterocyclic linking group is covalently bound a

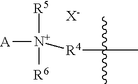

group through the R⁴ group, where the R⁴ group is an aliphatic group, R³ is an aliphatic group, and R⁵ and R⁶ are independently at each occurrence an aliphatic group or R⁵, R⁶, and the N of the

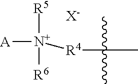

group taken together form a heterocyclic group, or R¹, R², and N taken together form an aliphatic group-bridged heterocyclic linking group (e.g., a quinuclidinium linking group), where a first terminus of the linking bridged heterocyclic group is covalently bound to the quaternary ammonium nitrogen of the compound and a second terminus of the aliphatic group-bridged heterocyclic linking group is covalently bound a

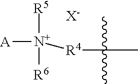

group, where the R⁴ group is an aliphatic group, R³ is an aliphatic group, and R⁵ and R⁶ are independently at each occurrence an aliphatic group or R⁵, R⁶, and the N of the

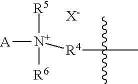

group taken together form a heterocyclic group; and X is chosen independently at each occurrence from halide anions (e.g., F⁻, Cl⁻, Br⁻ or I⁻), complex anions (e.g., BF₄⁻, SbF₆⁻, PF₆⁻, B(ArF₄)⁻, where ArF₄ is an aryl group substituted with four fluorine groups, B(Ar₄)⁻, where Ar is an aryl group, and the like), and the like, and any combination thereof.
Statement 2. A compound according to Statement 1, where the compound comprises (or has) the following structure:

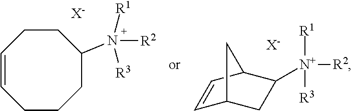

or the like.
Statement 3. A compound according to Statement 1 or 2, where R¹ and R² taken together with N form: a piperidinyl group, pyrrolidinyl group, azepanyl group, morpholinyl group, piperazinyl group, or 1,4-Diazabicyclo[2.2.2]octane (DABCO) group.

Statement 4. A compound according to any one of the preceding Statements, where R¹, R², and N taken together form a heterocyclic group and where the compound comprises (or has) the following structure:

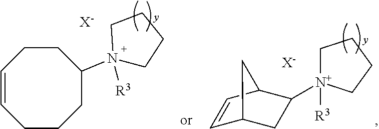

where y is 1, 2, 3, or 4.
Statement 5. A compound according to any one of the preceding Statements, where R¹ and R² taken together with N form a piperidinium group or R¹, R², and R³ taken together with N form an aliphatic-bridged piperidinium group (quinuclidinium group) and where the compound comprises (or has) the following structure:

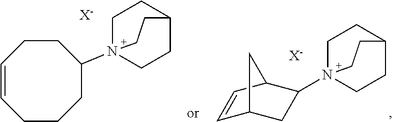

or the like.
Statement 6. A compound according to any one of the preceding Statements, where R¹ and R² taken together with N form a piperidinium group or R¹, R², and R³ taken together with N form an aliphatic-bridged heterocyclic group and where the compound comprises (or has) the following structure:

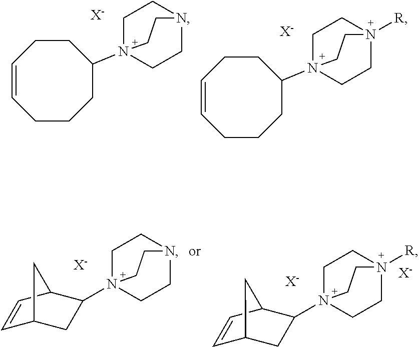

or the like, where R is an aliphatic group or an aryl group.
Statement 7. A compound according to any one of the preceding Statements, where the compound has the following structure:

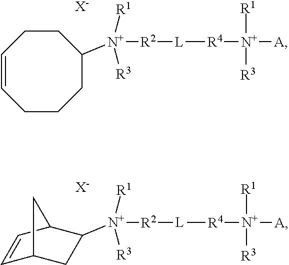

or the like
where L is a linking group and R⁴ is an aliphatic group.
Statement 8. A method of making a compound of the present disclosure (e.g., a compound of any one of the preceding claims), the method comprising: forming a first reaction mixture comprising: 1,5 cyclooctadiene, norbornadiene, or a combination thereof; one or more secondary amine(s), one or more hydroxyalkyl secondary amine(s), one or more multifunctional secondary amine(s) or any combination thereof; one or more H-atom transfer (HAT) catalyst(s); and one or more photocatalyst(s); subjecting the first reaction mixture to electromagnetic radiation comprising wavelengths from about 350 nm to about 700 nm to form one or more tertiary amine-functionalized cyclooctene compound(s), one or more tertiary amine-functionalized norbornene compound(s), one or more functionalized tertiary amine-functionalized cyclooctene compound(s), one or more functionalized tertiary amine-functionalized norbornene compound(s), one or more hydroxyalkyl amine-functionalized cyclooctene compound(s), one or more hydroxyalkylamine-functionalized norbornene compound(s), or any combination thereof; forming a second reaction mixture comprising: the tertiary amine-functionalized cyclooctene compound(s) and/or the tertiary amine-functionalized cyclooctene compound(s) and/or the functionalized tertiary amine-functionalized norbornene compound(s), or any combination thereof; one or more alkylating agent(s) (e.g., hydrocarbon halide(s), one or more trialkyloxonium salt(s)), or a combination thereof; or the hydroxyalkyl amine-functionalized cyclooctene compound(s), the hydroxyalkylamine-functionalized norbornene compound(s), functionalized tertiary amine-functionalized norbornene compound(s), or any combination thereof, one or more reductant(s); and one or more bromine source(s), where the second reaction mixture forms one or more compound of the present disclosure (e.g., one or more quaternary ammonium-functionalized cyclooctene compound(s), one or more quaternary ammonium-functionalized norbornene compound(s), one or more functionalized quaternary ammonium-functionalized cyclooctene compound(s), one or more functionalized quaternary ammonium-functionalized norbornene compound(s), or any combination thereof, when the second reaction mixture comprises the tertiary amine-functionalized cyclooctene compound(s), the tertiary amine-functionalized norbornene compound(s), the functionalized tertiary amine-functionalized cyclooctene compound(s), the one or more functionalized tertiary amine-functionalized norbornene compound(s), or any combination thereof and the one or alkylating agent(s); or one or more (bridged heterocyclic group)-functionalized cyclooctene compound(s), one or more (bridged heterocyclic group)-functionalized norbornene compound(s), or any combination thereof, when the second reaction mixture comprises the hydroxyalkyl amine-functionalized cyclooctene compound(s), the hydroxyalkylamine-functionalized norbornene compound(s), or any combination thereof, the reductant(s), and bromine source(s).
Statement 9. A method according to Statement 8, further comprising one or more or all of the following: isolating the tertiary amine-functionalized cyclooctene compound(s), the tertiary amine-functionalized norbornene compound(s), the functionalized tertiary amine-functionalized cyclooctene compound(s), the functionalized tertiary amine-functionalized norbornene compound(s) or any combination thereof, prior to formation of the second reaction mixture; or isolating the tertiary amine-functionalized cyclooctene compound or the (bridged piperidinium)-functionalized cyclooctene compound from the second reaction mixture.
Statement 10. A method according to Statement 8 or 9, where the one or more secondary amine(s) are chosen from pyrrolidines, azepanes, morpholines, piperazines, piperidin-4-amine, piperidin-4-ol, 1,4-Diazabicyclo[2.2.2]octane (DABCO), secondary alkyl amines (linear and branched), N-alkyl-N-aryl amines, N-methylcyclohexanamine, 2,2-imethoxy-N-methyl ethan-1-amine, N-methyl-2-(pyridine-4-yl)ethan-1-amine.
Statement 11. A method according to any one of Statements 8 or 9, where the secondary amines are multifunctional secondary amines.
Statement 12. A method according to any one of Statements 8 or 9, where the secondary amines are chosen from
N(H)R⁵R⁶, where R⁵ and R⁶ are independently chosen from aliphatic groups,

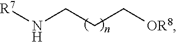

where R⁷ is an aliphatic group and R⁸ is H or an aliphatic group and n is 0 to 6,

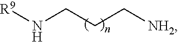

where R⁹ is an aliphatic group and n is 0 to 6,

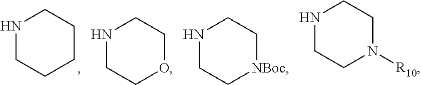

wherein R¹⁰ is an aliphatic group or an aryl group,

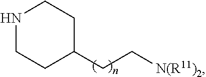

where the R¹¹ groups are both —H groups or both aliphatic groups
and n is 0 to 6,

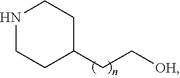

where n is 0 to 6,

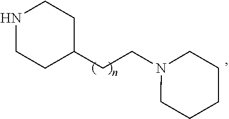

where n is 0 to 6,

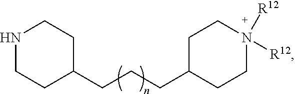

where n is 0 to 6 and R¹² is independently at each occurance an aliphatic group,

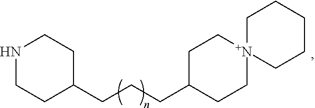

where n is 0 to 6,

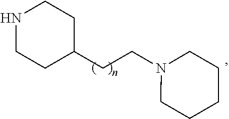

where n is 0 to 6; and

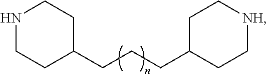

where n is 1 to 6, and any combination thereof.
13. A method according to any one of Statements 8 or 9, where the hydroxyalkyl secondary amine(s) are chosen from

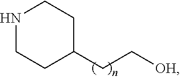

where n is 0 to 6, and any combination thereof and/or the multifunctional secondary amine(s) are chosen from

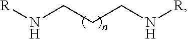

where n is 0 to 6 and is R independently, at each occurrence, is an aliphatic group,

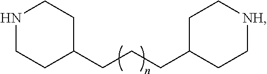

where n is 0 to 6, and the like, an any combination thereof.
Statement 14. A method according to any one of Statements 8-13, where the 1,5-cyclooctadiene and/or norbornadiene are present at about 0.25 molar equivalents to about 50 molar equivalents, based on the total moles of secondary amine(s), one or more hydroxyalkyl amine(s), or any combination thereof.
Statement 15. A method according to any one of Statements 8-14, where the secondary amine(s), the one or more hydroxyalkyl amine(s), or any combination thereof, are present at a concentration of from about 0.01 M to about 5 M (based on the total volume of the reaction mixture).
Statement 16. A method according to any one of Statements 8-15, where the H-atom transfer (HAT) catalyst is chosen from thiophenol, substituted thiophenols, phenol, substituted phenols, thiols, malonitrile, hydroxamic acid, silanes, and any combinations thereof.
Statement 17. A method according to any one of Statements 8-16, where the H-atom transfer (HAT) catalyst is present at about 0.1 mol % to about 500 mol %, based on the total moles of secondary amine(s), hydroxyalkyl amine(s), or any combination thereof.
Statement 18. A method according to any one of Statements 8-17, where the photocatalyst is chosen from iridium-bipyridine photocatalysts (e.g., Iridium(III) bis[2-(2,4-difluorophenyl)-5-methylpyridine-N,C20]-4,40-di-tert-butyl-2,20-bipyridine hexafluorophosphate ([Ir(dF(Me)ppy)₂(dtbbpy)]PF₆), and the like) acridinium photocatalysts (e.g., 9-mesityl-3,6-di-tert-butyl-10-phenylacridinium tetrafluoroborate and the like) and any combination thereof.
Statement 19. A method according to any one of Statements 8-19, where the photocatalyst is present at about 0.01 mol % to about 10 mol %, based on the total moles of pyridine, one or more hydroxyalkyl pyridine(s), or any combination thereof.
Statement 20. A method according to any one of Statements 8-19, further comprising one or both of the following: the first reaction mixture further comprises one or more solvent(s) chosen from non-polar solvents, cyclic ethers (such as, for example, tetrahydrofuran, dioxane and the like), aromatic solvents (such as, for example, benzene, toluene, xylene, and the like), aprotic solvents (such as, for example, acetonitrile, and the like), and the like, and any combination thereof, or the second reaction mixture comprises one or more solvent(s) chosen from cyclic ethers (such as, for example, tetrahydrofuran and the like), chlorinated solvents (such as, for example, dichloromethane, and the like), aprotic solvents (such as, for example, acetonitrile, and the like), and the like, and any combination thereof.
Statement 21. A method according to any one of Statements 8-20, where the first reaction mixture is subjected to the electromagnetic radiation for about 5 minutes to about 168 hours.
Statement 22. A method according to any one of Statements 8-21, where the first reaction mixture is subjected to the electromagnetic radiation at a temperature of about −50° C. to about 110° C.
Statement 23. A method according to any one of Statements 8-22, where the alkylating agent(s) is/are one or more hydrocarbon halide(s) is/are chosen from alkyl halides (e.g., C₁-C₂₀ alkyl halides), where the halide group is independently at each occurrence iodide (—I), bromide (—Br), chloride (—Cl), or the like, trialkyloxonium salts (e.g., C₁-C₂₀ trialkyloxonium salts), where the anion of the trialkyloxonium salt(s) is independently at each occurance chosen from complex anions and the like, and any combination thereof.
Statement 24. A method according to any one of Statements 8-23, where the alkyl halide(s) and/or trialkyloxonium salt(s) is/are present at about 0.1 to about 50 molar equivalents based on the total moles of precursor compound(s) (e.g., tertiary amine-functionalized precursor compound(s), such as, for example, the tertiary amine-functionalized cyclooctene compound(s) and/or the tertiary amine-functionalized norbornene compound(s), or the like, or any combination thereof).
Statement 25. A method according to any one of Statements 8-24, further comprising: heating or holding the second reaction mixture to a temperature of from about −50° C. to about 120° C., when the second reaction mixture comprises the tertiary amine-functionalized cyclooctene compound(s) and/or the tertiary amine-functionalized cyclooctene compound(s) and/or the functionalized tertiary amine-functionalized cyclooctene compound(s), or any combination thereof, and the alkylating agent(s); or cooling or holding the second reaction mixture to a temperature of from about −20° C. to about 80° C., when the second reaction mixture comprises the one or more hydroxyalkyl amine-functionalized cyclooctene compound(s), one or more hydroxyalkylamine-functionalized norbornene compound(s), or any combination thereof, the reductant(s), and bromine source(s).
Statement 26. A polymer comprising the following structure:

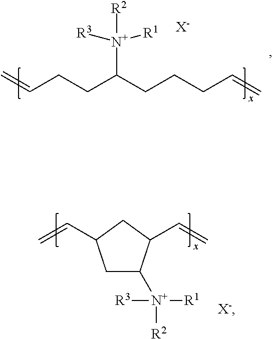

or the like, or any combination thereof, where R¹, R², and R³ are independently at each occurrence an aliphatic group, or R¹, R², and N taken together form a heterocyclic group and R³ is an aliphatic group, or R¹, R², R³, and N taken together form an aliphatic group-bridged heterocyclic group (e.g., a quinuclidinium group), or R³ comprises a linking group and a

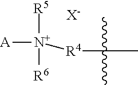

group, where a first terminus of the linking group is covalently bound to the quaternary ammonium nitrogen of the compound and a second terminus of the linking group is covalently bound the

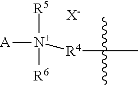

group through the R⁴ group, and R¹ and R² are independently at each occurrence an aliphatic group or R¹, R², and N taken together form a heterocyclic group, or R¹ and/or R² each comprises a linking group and a

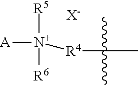

group, where a first terminus of the linking group is covalently bound to the quaternary ammonium nitrogen of the compound and a second terminus of the linking group is covalently bound the

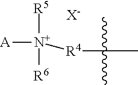

group through the R⁴ group, where the R⁴ group is an aliphatic group, and R³ is an aliphatic group, or R¹, R², and N taken together form a heterocyclic linking group, where a first terminus of the heterocyclic linking group is covalently bound to the quaternary ammonium nitrogen of the compound and a second terminus of the heterocyclic linking group is covalently bound a

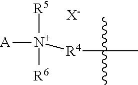

group through the R⁴ group, where the R⁴ group is an aliphatic group, and R³ is an aliphatic group, or R¹, R², and N taken together form an aliphatic group-bridged heterocyclic linking group (e.g., a quinuclidinium linking group), where a first terminus of the linking bridged heterocyclic group is covalently bound to the quaternary ammonium nitrogen of the compound and a second terminus of the aliphatic group-bridged heterocyclic linking group is covalently bound a

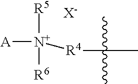

group, where the R⁴ group is an aliphatic group, and R³ is an aliphatic group; and X is chosen independently at each occurrence from halide anions (e.g., F⁻, Cl⁻, Br⁻ or I⁻), complex anions (e.g., BF₄⁻, SbF₆⁻, PF₆⁻, B(ArF₄)⁻, where ArF₄ is an aryl group substituted with four fluorine groups, B(Ar₄)⁻, where Ar is an aryl group, and the like), and the like; and x is the mol fraction of structure(s) in the polymer and the mol fraction of structures is about 0.01 to about 1, including all 0.005 mol fraction values and ranges therebetween.
Statement 27. A polymer according to Statement 26, where the polymer comprises (or has) the following structure:

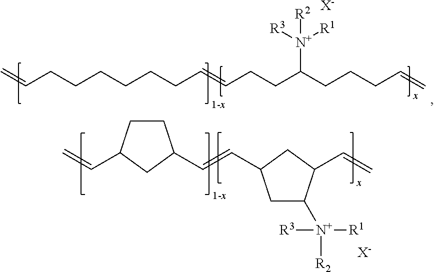

or the like.
Statement 28. A polymer according to Statement 26 or 27, where the polymer has a molecular weight (Mw and/or Mn) of 500 g/mol to 1,000,000 g/mol (e.g., 5,000 g/mol to 250,000 g/mol).
Statement 29. A polymer according to any one of Statements 26-28, where at least a portion of the terminal carbons is, independently at each occurrence, substituted with an aryl group, hydrogen, an alkyl group, a halogen, a hydroxyl group, and/or at least a portion of the terminal carbons is, independently at each occurrence, a carbonyl carbon of an aldehyde group, a ketone group, an acid group, or acetate group.
Statement 30. A polymer comprising the following structure (repeat unit):

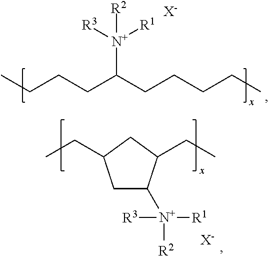

or any combination thereof, where R¹, R², and R³ are independently at each occurrence an aliphatic group, or R¹, R², and N taken together form a heterocyclic group and R³ is an aliphatic group, or R¹, R², R³, and N taken together form an aliphatic group-bridged heterocyclic group (e.g., a quinuclidinium group), or R³ comprises a linking group and a

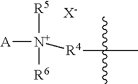

group, where a first terminus of the linking group is covalently bound to the quaternary ammonium nitrogen of the compound and a second terminus of the linking group is covalently bound the

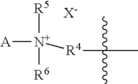

group through the R⁴ group, and R¹ and R² are independently at each occurrence an aliphatic group or R¹, R², and N taken together form a heterocyclic group, or R¹ and/or R² each comprises a linking group and a

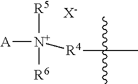

group, where a first terminus of the linking group is covalently bound to the quaternary ammonium nitrogen of the compound and a second terminus of the linking group is covalently bound the

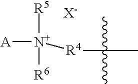

group through the R⁴ group, where the R⁴ group is an aliphatic group, and R³ is an aliphatic group, or R¹, R², and N taken together form a heterocyclic linking group, where a first terminus of the heterocyclic linking group is covalently bound to the quaternary ammonium nitrogen of the compound and a second terminus of the heterocyclic linking group is covalently bound a

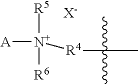

group through the R⁴ group, where the R⁴ group is an aliphatic group, and R³ is an aliphatic group, or R¹, R², and N taken together form an aliphatic group-bridged heterocyclic linking group (e.g., a quinuclidinium linking group), where a first terminus of the linking bridged heterocyclic group is covalently bound to the quaternary ammonium nitrogen of the compound and a second terminus of the aliphatic group-bridged heterocyclic linking group is covalently bound a

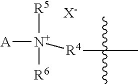

group, where the R⁴ group is an aliphatic group, and R³ is an aliphatic group; X is chosen independently at each occurrence from halide anions (e.g., F⁻, Cl⁻, Br⁻ or I⁻), complex anions (e.g., BF₄⁻, SbF₆⁻, PF₆⁻, B(ArF₄)⁻, where ArF₄ is an aryl group substituted with four fluorine groups, B(Ar₄)⁻, where Ar is an aryl group, and the like), and the like, and any combination thereof); and x is the mol fraction of the structure(s) (repeat unit(s)) in the polymer and the mol fraction of structure(s) (repeat unit(s)) is/are about 0.01 to about 1, including all 0.005 mol fraction values and ranges therebetween.
Statement 31. A polymer according to Statement 30, where the polymer comprises the following structure:

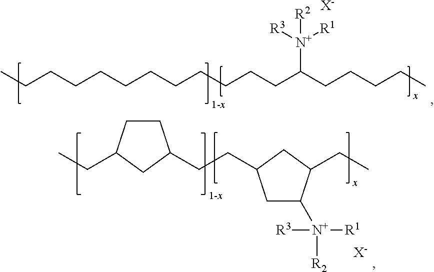

or the like.
Statement 32. A polymer according to Statement 30 or 31, where the polymer has a molecular weight (Mw and/or Mn) of 500 g/mol to 1,000,000 g/mol (e.g., 5,000 g/mol to 250,000 g/mol).
Statement 33. A polymer according to any one of Statements 30-32, where at least a portion of the terminal carbons is, independently at each occurrence, substituted with an aryl group, hydrogen, an alkyl group, a halogen, a hydroxyl group, and/or at least a portion of the terminal carbons is, independently at each occurrence, a carbonyl carbon of an aldehyde group, a ketone group, an acid group, or acetate group.
Statement 34. A method of making a polymer of the present disclosure (e.g., a polymer of any one of Statements 26-33, such as, for example, a polymer comprising following structure:

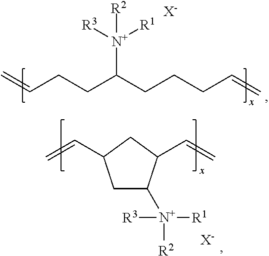

or any combination thereof, where R¹, R², and R³ are independently at each occurrence an aliphatic group, or R¹, R², and N taken together form a heterocyclic group and R³ is an aliphatic group, or R¹, R², R³, and N taken together form an aliphatic group-bridged heterocyclic group (e.g., a quinuclidinium group), or R³ comprises a linking group and a

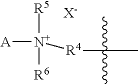

group, where a first terminus of the linking group is covalently bound to the quaternary ammonium nitrogen of the compound and a second terminus of the linking group is covalently bound the

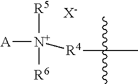

group through the R⁴ group, and R¹ and R² are independently at each occurrence an aliphatic group or R¹, R², and N taken together form a heterocyclic group, or R¹ and/or R² each comprises a linking group and a

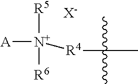

group, where a first terminus of the linking group is covalently bound to the quaternary ammonium nitrogen of the compound and a second terminus of the linking group is covalently bound the

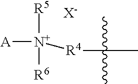

group through the R⁴ group, where the R⁴ group is an aliphatic group, and R³ is an aliphatic group, or R¹, R², and N taken together form a heterocyclic linking group, where a first terminus of the heterocyclic linking group is covalently bound to the quaternary ammonium nitrogen of the compound and a second terminus of the heterocyclic linking group is covalently bound a

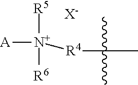

group through the R⁴ group, where the R⁴ group is an aliphatic group, and R³ is an aliphatic group, or R¹, R², and N taken together form an aliphatic group-bridged heterocyclic linking group (e.g., a quinuclidinium linking group), where a first terminus of the linking bridged heterocyclic group is covalently bound to the quaternary ammonium nitrogen of the compound and a second terminus of the aliphatic group-bridged heterocyclic linking group is covalently bound a

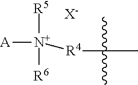

group, where the R⁴ group is an aliphatic group, and R³ is an aliphatic group, X is chosen independently at each occurrence from halide anions (e.g., F⁻, Cl⁻, Br⁻ or I⁻), complex anions (e.g., BF₄⁻, SbF₆⁻, PF₆⁻, B(ArF₄)⁻, where ArF₄ is an aryl group substituted with four fluorine groups, B(Ar₄)⁻, where Ar is an aryl group, and the like), and the like, and any combination thereof, and x is the mol fraction of the structure(s) (repeat unit(s)) in the polymer and the mol fraction of structure(s) (repeat unit(s)) is/are about 0.01 to about 1, including all 0.005 mol fraction values and ranges therebetween), the method comprising polymerizing (in a ring-opening-metathesis polymerization (ROMP)) one or more monomer(s) independently chosen from monomer(s) having the following structure:

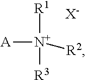

where A is a cyclooctenyl group or a norbornenyl group; R¹, R², and R³ are independently at each occurrence an aliphatic group, or R¹ and R² are taken together with N to form a heterocyclic group and R¹ is an aliphatic group, or R¹, R², and R³ are taken together with N to form bridged heterocyclic group (e.g., a quinuclidinium group); X is chosen from halides, BF₄⁻, SbF₆⁻, PF₆⁻, B(ArF₄)⁻, where ArF₄ is an aryl group substituted with four fluorine groups, B(Ar₄)⁻, where Ar is an aryl group, or copolymerizing one or more monomer(s) independently chosen from monomer(s) having the following structure:

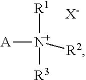

where A is a cyclooctenyl group or a norbornenyl group; R¹, R², and R³ are independently at each occurrence an aliphatic group, or R¹, R², and N taken together form a heterocyclic group and R³ is an aliphatic group, or R¹, R², R³, and N taken together form an aliphatic group-bridged heterocyclic group (e.g., a quinuclidinium group), or R³ comprises a linking group and a

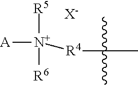

group, where a first terminus of the linking group is covalently bound to the quaternary ammonium nitrogen of the compound and a second terminus of the linking group is covalently bound the

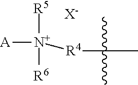

group through the R⁴ group, and R¹ and R² are independently at each occurrence an aliphatic group or R¹, R², and N taken together form a heterocyclic group, or R¹ and/or R² each comprises a linking group and a

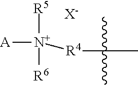

group, where a first terminus of the linking group is covalently bound to the quaternary ammonium nitrogen of the compound and a second terminus of the linking group is covalently bound the

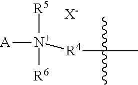

group through the R⁴ group, where the R⁴ group is an aliphatic group, and R³ is an aliphatic group, or R¹, R², and N taken together form a heterocyclic linking group, where a first terminus of the heterocyclic linking group is covalently bound to the quaternary ammonium nitrogen of the compound and a second terminus of the heterocyclic linking group is covalently bound a

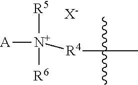

group through the R⁴ group, where the R⁴ group is an aliphatic group, and R³ is an aliphatic group, or R¹, R², and N taken together form an aliphatic group-bridged heterocyclic linking group (e.g., a quinuclidinium linking group), where a first terminus of the linking bridged heterocyclic group is covalently bound to the quaternary ammonium nitrogen of the compound and a second terminus of the aliphatic group-bridged heterocyclic linking group is covalently bound a

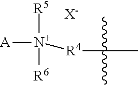

group, where the R⁴ group is an aliphatic group, and R³ is an aliphatic group; X is chosen independently at each occurrence from halide anions (e.g., F⁻, Cl⁻, Br⁻ or I⁻), complex anions (e.g., BF₄⁻, SbF₆⁻, PF₆⁻, B(ArF₄)⁻, where ArF₄ is an aryl group substituted with four fluorine groups, B(Ar₄)⁻, where Ar is an aryl group, and the like), and the like, and any combination thereof) and one or more co-monomer(s) chosen from unsaturated hydrocarbon monomers (e.g., cyclooctadiene, norbornadiene, cis-cyclooctene, trans-cyclooctene, norbornene, cyclopentene, cyclobutene, and the like, and any combination thereof).
Statement 35. A method according to Statement 34, where the polymer comprises (or has) the following structure:

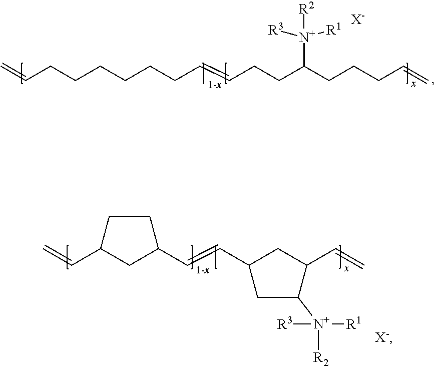

or the like.
Statement 36. A method according to Statement 34 or 35, further comprising at least partially, substantially, or completely hydrogenating the polymer.
Statement 37. A method according to Statement 36, where the hydrogenated polymer comprises (or has) the following structure:

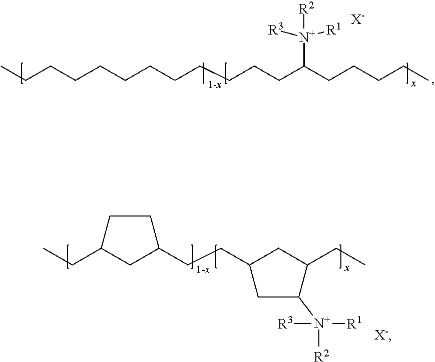

or the like.
Statement 38. An anion exchange membrane comprising one or more polymer(s) of the present disclosure (e.g., polymer(s) according to any one of Statements 26-33 or made by a method of the present disclosure, such as, for example, polymer(s) comprising the following structure:

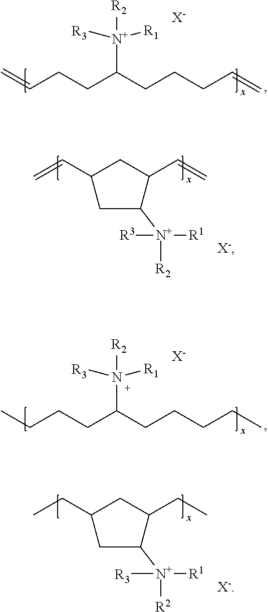

or the like, or any combination thereof, where R¹, R², and R³ are independently at each occurrence an aliphatic group, or R¹, R², and N taken together form a heterocyclic group and R³ is an aliphatic group, or R¹, R², R³, and N taken together form an aliphatic group-bridged heterocyclic group (e.g., a quinuclidinium group), or R³ comprises a linking group and a

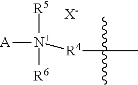

group, where a first terminus of the linking group is covalently bound to the quaternary ammonium nitrogen of the compound and a second terminus of the linking group is covalently bound the

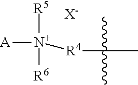

group through the R⁴ group, and R¹ and R² are independently at each occurrence an aliphatic group or R¹, R², and N taken together form a heterocyclic group, or R¹ and/or R² each comprises a linking group and a

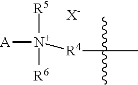

group, where a first terminus of the linking group is covalently bound to the quaternary ammonium nitrogen of the compound and a second terminus of the linking group is covalently bound the

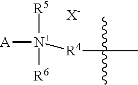

group through the R⁴ group, where the R⁴ group is an aliphatic group, and R³ is an aliphatic group, or R¹, R², and N taken together form a heterocyclic linking group, where a first terminus of the heterocyclic linking group is covalently bound to the quaternary ammonium nitrogen of the compound and a second terminus of the heterocyclic linking group is covalently bound a

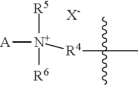

group through the R⁴ group, where the R⁴ group is an aliphatic group, and R³ is an aliphatic group, or R¹, R², and N taken together form an aliphatic group-bridged heterocyclic linking group (e.g., a quinuclidinium linking group), where a first terminus of the linking bridged heterocyclic group is covalently bound to the quaternary ammonium nitrogen of the compound and a second terminus of the aliphatic group-bridged heterocyclic linking group is covalently bound a

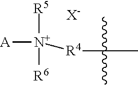

group, where the R⁴ group is an aliphatic group, and R³ is an aliphatic group; X is chosen independently at each occurrence from halide anions (e.g., F⁻, Cl⁻, Br⁻ or I⁻), complex anions (e.g., BF₄⁻, SbF₆⁻, PF₆⁻, B(ArF₄)⁻, where ArF₄ is an aryl group substituted with four fluorine groups, B(Ar₄)⁻, where Ar is an aryl group, and the like), and the like, and any combination thereof); and
x is the mol fraction of the structure(s) (repeat unit(s)) in the polymer and the mol fraction of structure(s) (repeat unit(s)) is/are about 0.01 to about 1, including all 0.005 mol fraction values and ranges therebetween) and optionally, one or more salt(s) (e.g., lithium salt(s), such as, for example, lithium hexafluorophosphate, lithium bis(trifluoromethanesulfonyl)imide (LiTFSI), and the like), or the like, or any combination thereof.
Statement 39. An anion exchange membrane according to Statement 38, where the one or more polymer(s) comprise (or have) the following structure:

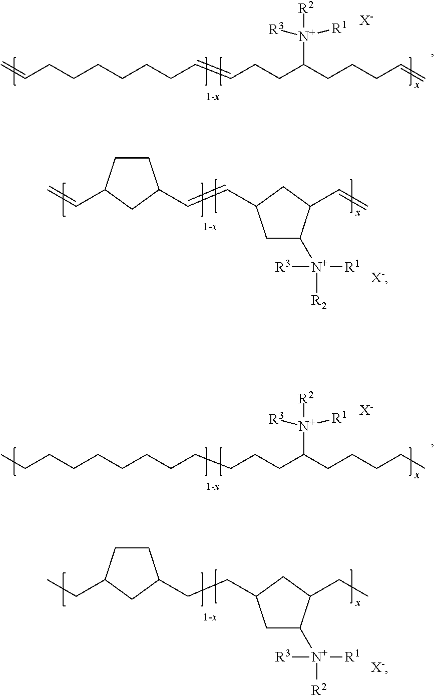

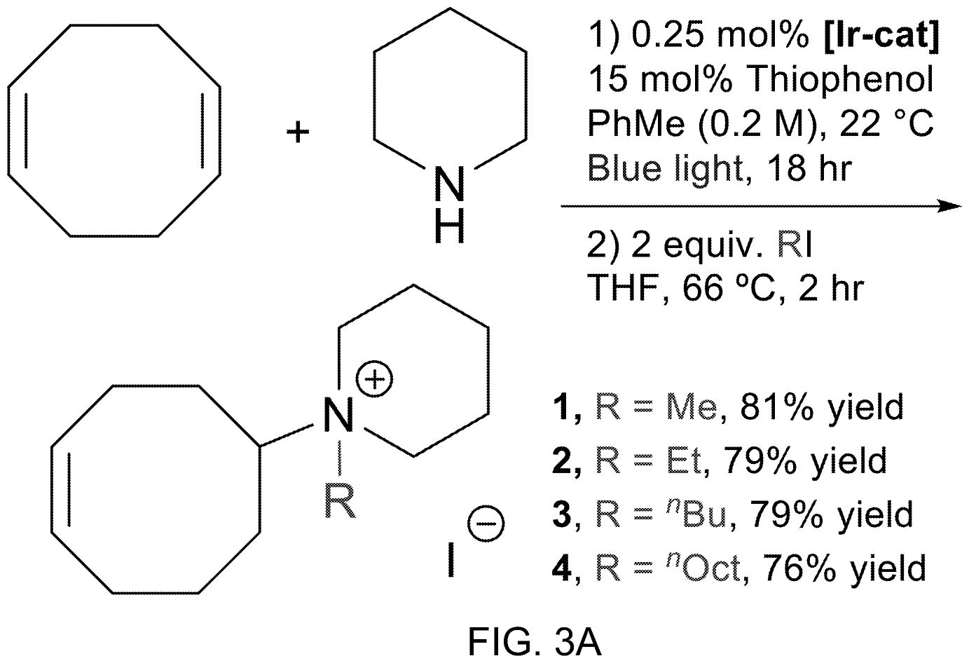

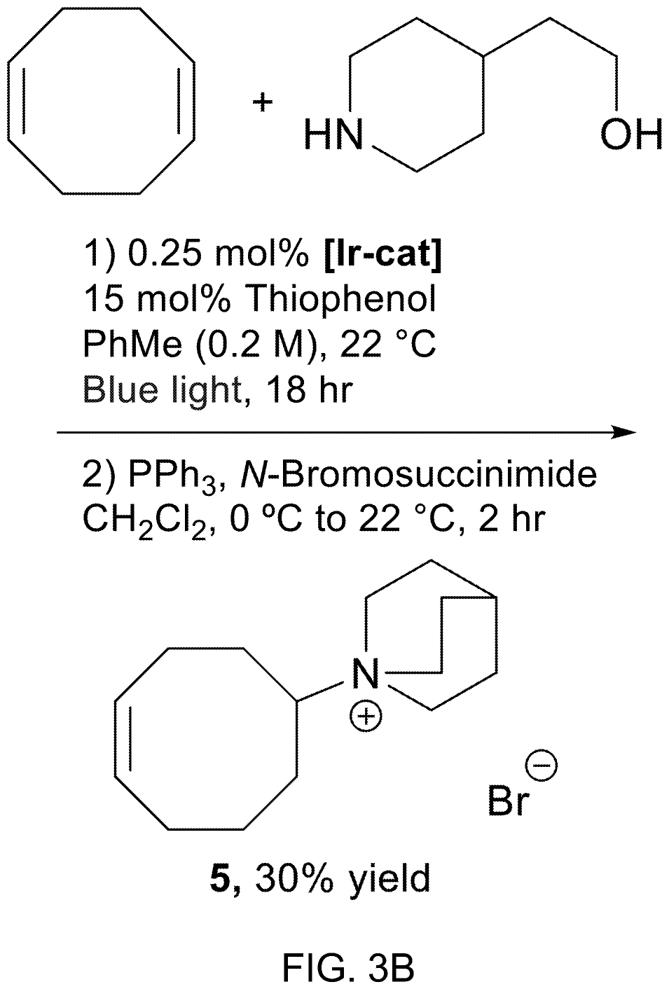

or the like, or any combination thereof.
Statement 40. An anion exchange membrane according to Statement 38 or 39, where the membrane has a thickness of about 1 micron to about 500 microns.
Statement 41. An anion exchange membrane according to any one of Statements 38-40, where the membrane exhibits one or more or all of the following: a hydroxide conductivity (a (OH—, at about 22° C.)) of from about 1 mS/cm to about 60 mS/cm; a water uptake (WU) of from about 5% to about 200%; a dimensional change (ΔL) of from about 0% to about 30%; an ion exchange capacities (IECs) of from about 0.08 mmol I⁻/g to about 3 mmol I⁻/g; a stress at break of from about 1 MPa to about 30 MPa; a strain at break of from about 100% to about 1600%; or a retained conductivity (e.g., in 1M KOH, at about 80° C., for about 30 days) of from about 0% to about 100%.
Statement 42. An anion exchange membrane according to any one of Statements 38-41, where the membrane is formed by a method comprising crosslinking/reactive casting, solution casting, annealing, melt pressing, or the like.
Statement 43. A device of the present disclosure comprising one or more anion exchange membrane(s) of the present disclosure (e.g., one or more anion exchange membrane(s) of any one of Statements 38-42).
Statement 44. A device according to Statement 43, where the device is an energy-storage device, an energy-generating device, or the like.
Statement 45. A device according to Statement 43 or 44, where the electrochemical device is a battery, a fuel cell, a water-electrolysis device, or the like.
Statement 46. A process for generating a piperidinium-functionalized cyclooctene monomer, the process comprising: combining 1,5 cyclooctadiene, piperidine, thiophenol and a photocatalyst to form a first reaction mixture; subjecting the first reaction mixture to light to form a second reaction mixture; adding an alkyl halide to the second reaction mixture to form a third reaction mixture; and heating the third reaction mixture to generate piperidinium-functionalized cyclooctene monomer.
Statement 47. The process of Statement 1, further comprising isolating the piperidinium-functionalized cyclooctene monomer.
Statement 48. The process of any one of Statements 1-2, where the photocatalyst comprises [Ir(dF(Me)ppy)₂(dtbbpy)]PF₆(Iridium(III) bis[2-(2,4-difluorophenyl)-5-methylpyridine-N,C₂₀]-4,40-di-tert-butyl-2,20-bipyridine hexafluorophosphate).
Statement 49. The process of any one of Statements 1-3, where the first reaction mixture comprises 0.25 mol % photocatalyst.
Statement 50. The process of any one of Statements 1-4, where the first reaction mixture comprises 15 mol % thiophenol.
Statement 51. The process of any one of Statements 1-5, where the first reaction mixture comprises PhMe.
Statement 52. The process of any one of Statements 1-6, where the light is blue light.
Statement 53. The process of any one of Statements 1-7, where the first reaction mixture is subjected light for 8-28 h.
Statement 54. The process of any one of Statements 1-8, where the alkyl halide is added at greater than 1, greater than 1.5, greater than 2.0, or greater than 2.5 molar equivalent.
Statement 55. The process of any one of Statements 1-9, where the alkyl halide is selected from the group consisting of methyl halide, ethyl halide, n-butyl halide, and n-octyl halide.
Statement 56. The process of any one of Statements 1-9, where the alkyl halide is selected from the group consisting of methyl iodide, ethyl iodide, n-butyl iodide, and n-octyl iodide
Statement 57. The process of any one of Statements 1-11, where THF is added to the second reaction mixture.
Statement 58. The process of any one of Statements 1-12, where the third reaction mixture is heated from 50-100° C.
Statement 59. The process of any one of Statements 1-13, where the third reaction mixture is heated from 60-70° C.
Statement 60. The process of any one of Statements 1-14, where the third reaction mixture is heated at 66° C.
Statement 61. The process of any one of Statements 1-15, further comprising generating a piperidinium-functionalized alkaline anion exchange membrane from the piperidinium-functionalized cyclooctene monomer.
Statement 62. A process for generating a piperidinium-functionalized cyclooctene monomer, the process comprising the process as outline in FIG. 3B.
Statement 62. The process of Statement 17, further comprising generating a piperidinium-functionalized alkaline anion exchange membrane from the piperidinium-functionalized cyclooctene monomer.
Statement 64. A piperidinium-functionalized alkaline anion exchange membrane as generated by the process of Statement 16.
Statement 65. A piperidinium-functionalized alkaline anion exchange membrane as generated by the process of Statement 17.

The steps of the methods described in the various examples disclosed herein are sufficient to carry out a method of the present disclosure. Thus, in various examples, a method consists essentially of a combination of the steps of the methods disclosed herein. In various other examples, a method consists of such steps.

The following examples are presented to illustrate the present disclosure. They are not intended to be limiting in any matter.

### EXAMPLE 1

The following is an example of: piperidinium-functionalized cyclooctene monomers and quinuclidinium-functionalized cyclooctene monomers of the present disclosure, and methods of making and using said monomers; piperidinium-functionalized polyethylenes (PEMs) of the present disclosure and quinuclidinium-functionalized polyethylenes (PEQs) of the present disclosure, and methods of making and using said PEMs and PEQs; and alkaline anion exchange membranes (AAEMs) of the present disclosure, methods of making and using said AAEMs.

Alkaline anion exchange membranes (AAEMs) with high hydroxide conductivity and good alkaline stability are essential for the development of anion exchange membrane fuel cells to generate clean energy by converting renewable fuels to electricity. Polyethylene-based AAEMs with excellent properties can be prepared via sequential ring-opening metathesis polymerization (ROMP) and hydrogenation of cyclooctene derivatives. However, one of the major limitations of this approach is the complicated multi-step synthesis of functionalized cyclooctene monomers. It was found that piperidinium-functionalized cyclooctene monomers can be easily prepared via the photocatalytic hydroamination of cyclooctadiene with piperidine in a one-pot, two-step process to produce high-performance AAEMs.

Most synthetic routes to these COE monomers first entail an oxidation state or functional group manipulation of COD, followed by the installation of a nitrogen group. A more direct and conceptually simple synthesis of these compounds would be the redox neutral hydroamination of COD. While significant progress in the field has been made, intermolecular hydroaminations of unactivated alkenes with simple amines remain rare. Indeed, only a single example of COD hydroamination has been published to date. In 2017, it was reported that the photocatalytic hydroamination of COD with piperidine could afford a functionalized COE in a single step. With the robust ROMP/hydrogenation strategy, it was sought to convert these easily accessible monomers to potential AAEM materials. A modified protocol was used to prepare a series of piperidinium-functionalized COE monomers in a two-step, one-pot process that directly combines COD, piperidine, and an alkyl iodide. This photocatalytic method allows for the efficient synthesis of multi-gram quantities of monomers in a 100% atom-economical fashion. The quinuclidinium-functionalized analogue can also be synthesized in two steps from commercially available starting materials. It is believed that this is the first report of aromatic-free piperidinium- and quinuclidinium-functionalized AAEMs.

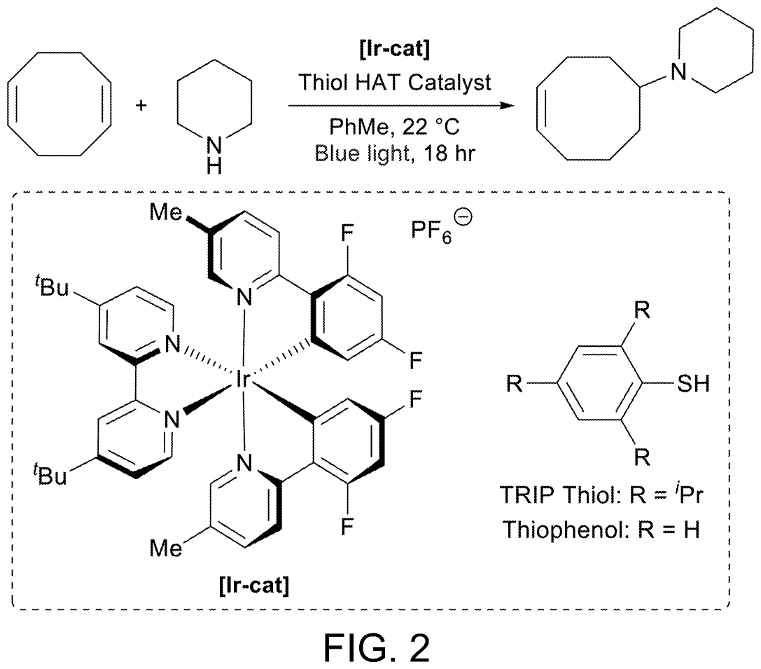

Optimization of monomer synthesis. In a previous report on the photocatalytic hydroamination of olefins, it was disclosed that COD could be directly functionalized with piperidine. Although the reported conditions were noteworthy for their high efficiency across a broad scope of substrates, a number of factors were identified that made them less ideal for the large-scale synthesis of AAEM monomers, such as the high loading of photocatalyst [Ir(dF(Me)ppy)₂(dtbbpy)]PF₆ ([Ir-cat], 2 mol %), the use of commercially unavailable 2,4,6-triisopropylbenzenethiol (TRIP thiol) as H-atom transfer (HAT) catalyst (50 mol %), and the relatively dilute reaction conditions (0.05 M). Seeking to develop an efficient multi-gram synthesis of cyclooctene-piperidine monomers, optimization studies were commenced (FIG. 2, Table 1).

TABLE 1
Optimization of 1,5-cyclooctadiene
(COD) hydroamination reactions.

| | | COD | [Ir-cat] | | |
|---|---|---|---|---|---|
| Entry | [Piperidine] | Equiv. | mol % | Thiol (mol %) | Yield |
| 1 | 0.05M | 5.0 | 2.0 | TRIP Thiol (50) | 76% |
| 2 | 0.05M | 10 | 2.0 | TRIP Thiol (50) | 82% |
| 3 | 0.2M | 10 | 2.0 | TRIP Thiol (50) | 72% |
| 4 | 0.2M | 10 | 0.25 | TRIP Thiol (50) | 86% |
| 5 | 0.2M | 10 | 0.25 | TRIP Thiol (15) | 80% |
| 6 | 0.2M | 10 | 0.25 | Thiophenol (15) | 69% |
| 7 | 0.2M | 10 | 0.25 | Thiophenol (50) | 64% |

Reactions were run at 0.5 mmol scale (piperidine). Yields were calculated based on piperidine for isolated materials after purification.

Using the original conditions, the desired addition product was observed in 76% yield based on piperidine (entry 1), with the major side product resulting from double hydroamination of the alkenes in COD. Increasing the equivalents of COD from 5 to 10 gave a modestly improved yield and markedly decreased the over-hydroamination side products (entry 2). Increasing the reaction concentration from 0.05 M to 0.2 M in toluene reduced the reactivity of the system (entry 3), but an accompanying reduction in photocatalyst loading boosted the yield to 86% (entry 4). Notably, the reaction still worked efficiently with only 15 mol % HAT catalyst (entry 5). Switching to thiophenol as a simpler HAT catalyst proved to be slightly less efficient (entry 6), but in weighing the practicality of the reaction this system was selected moving forward.

The possibility of a telescoped sequence was then evaluated to directly isolate the quaternary ammonium monomers. After the optimized hydroamination reaction described above (Table 1, entry 6), the toluene, unreacted piperidine, COD, and thiophenol catalyst were removed under reduced pressure. The residue was refluxed with 2 equiv. of Mel in THF for 2 hours providing white solid 1 in 81% yield after trituration in Et₂O. This one-pot, two-step approach can be used to prepare monomers expediently in at least 1.5-gram scale. Similar strategies were applied to synthesize other piperidinium-functionalized monomers, including ethyl (2), n-butyl (3), and n-octyl (4) derivatives (FIG. 3A). Quinuclidinium monomer 5 was prepared in 30% yield over two steps via hydroamination of COD with 2-(piperidin-4-yl)ethanol followed by an Appel reaction (FIG. 3B).

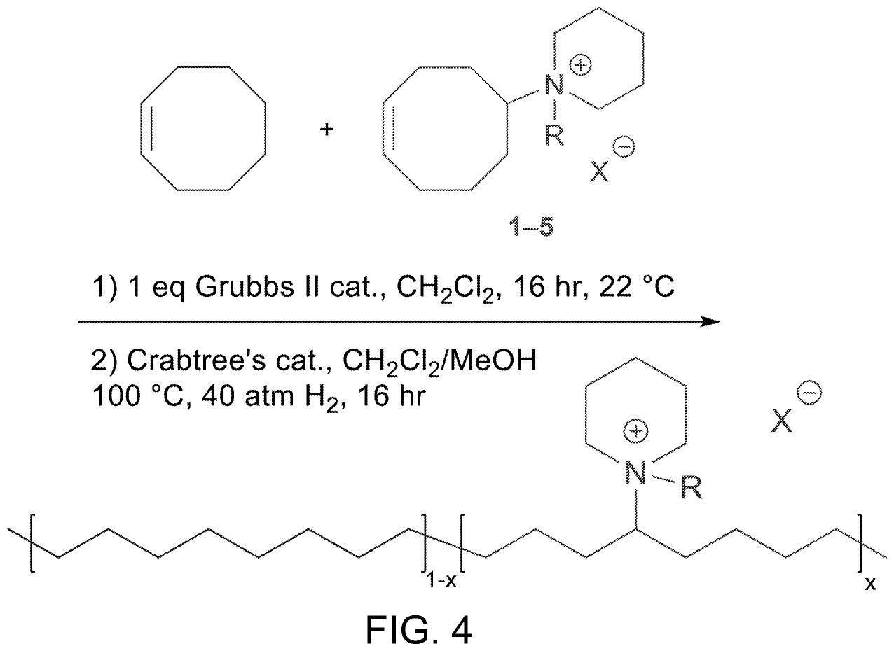

Membrane synthesis and properties. With various functionalized cyclooctene monomers in hand, a series of piperidinium-functionalized AAEMs (i.e. PEPM, PEPE, PEPB, PEPO, and PEQ membranes from monomers 1-5, respectively) were prepared via a ROMP/hydrogenation sequence (FIG. 4, Table 2).

TABLE 2
Summary of AAEM properties.

| | | | IEC | σ | | |
|---|---|---|---|---|---|---|
| | Ionic | mol % | (mmol | (OH⁻, 22° C.) | | |
| Samples | Monomer | of 1-5 | I⁻/g)^{a} | (mS/cm)^{b} | WU^{c} | ΔL^{d} |
| PEPM_{0.16} | 1 | 16% | 1.09 | 26 ± 1 | 22% | 4% |
| PEPM_{0.20} | 1 | 20% | 1.31 | 35 ± 2 | 26% | 7% |
| PEPM_{0.23} | 1 | 23% | 1.41 | 43 ± 2 | 54% | 8% |
| PEPM_{0.28} | 1 | 28% | 1.61 | 53 ± 2 | 92% | 9% |
| PEPE_{0.20} | 2 | 20% | 1.25 | 36 ± 2 | 28% | 6% |
| PEPB_{0.18} | 3 | 18% | 1.12 | 16 ± 1 | 24% | 6% |
| PEPO_{0.23} | 4 | 23% | 1.23 | 15 ± 5 | 40% | 8% |
| PEQ_{0.20} | 5 | 20% | 1.25 | 35 ± 1 | 36% | 11% |

^{a}Determined by ¹H NMR analysis before hydrogenation.
^{b}Determined from the average of three trials ± standard deviation.
^{c}Water uptake at 22° C. = 100 × [mass_{wet} − mass_{dry}]/mass_{dry}%.
^{d}Dimensional change at 22° C. = 100 × [length_{wet} − length_{dry}]/length_{dry}%.

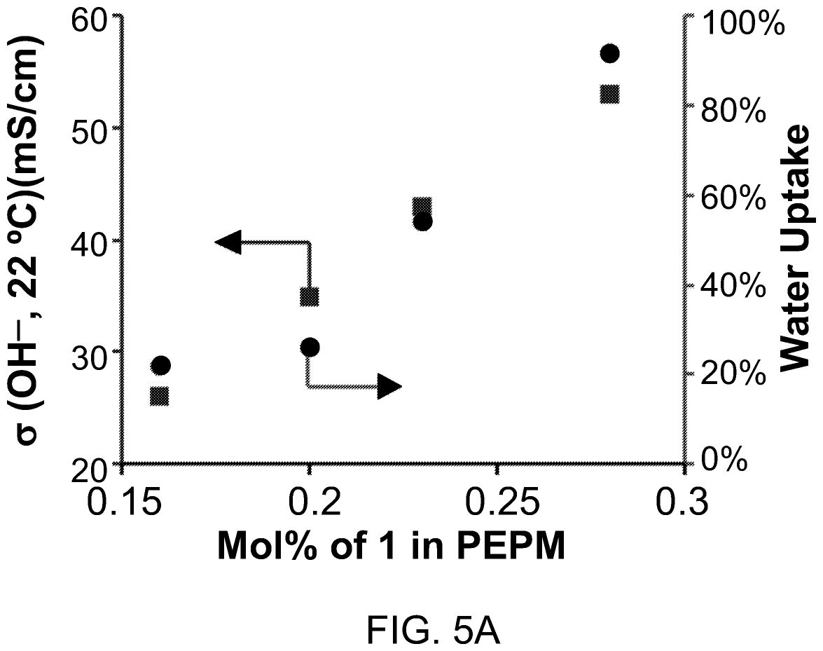

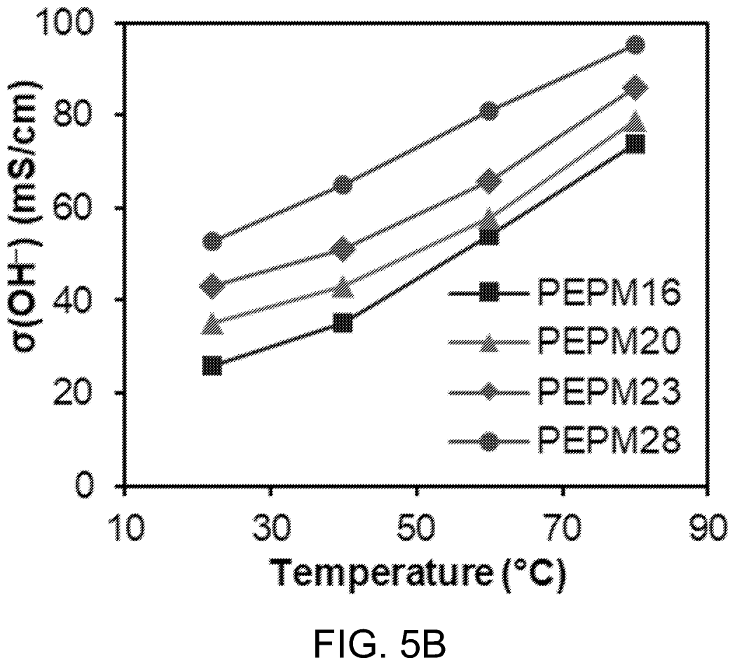

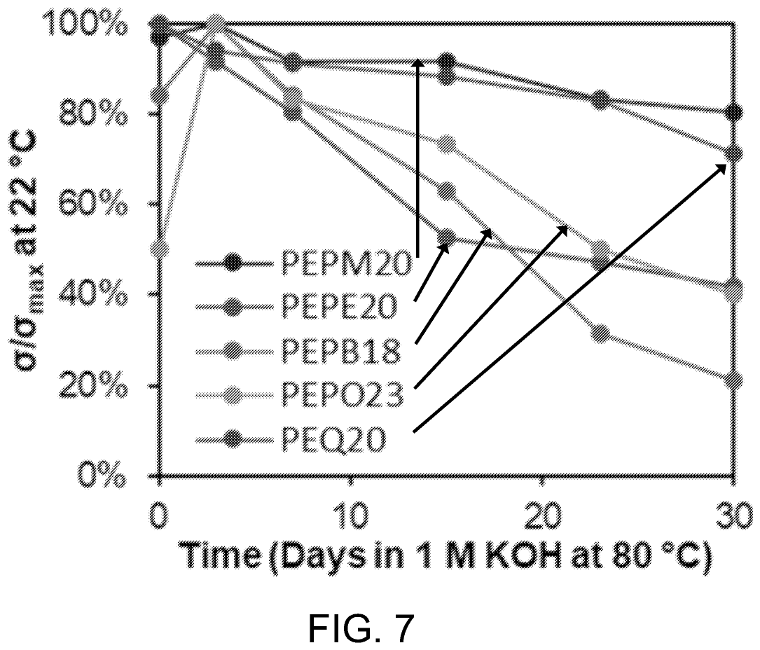

The functionalized monomers were first copolymerized with cis-COE by adapting our previous ROMP procedures. The molar ratio of cis-COE to Grubbs II catalyst was 800:1, and the incorporation of the ionic monomers can be easily tuned by changing their loading (Table 2). The resulting copolymers were then hydrogenated using Crabtree's catalyst and H₂ to produce AAEMs with PE backbones. After hydrogenation, the AAEM thin films were casted via a melt-pressing method, and the anions were exchanged to hydroxide in a 1 M KOH aqueous solution before measuring hydroxide conductivity (σ(OH⁻, 22° C.)), water uptake (WU), and dimensional change (ΔL) (Table 2). Their ion exchange capacities (IECs) were determined by comparing integration ratios in 1H NMR spectra (Table 2), and the results were consistent with monomer feeding ratios and Mohr titration results (see Table 7). When R=Me, both the hydroxide conductivity and WU increased with IEC, and it was found that the cation incorporation ratio in the range of 20-23% allowed for a good balance between the two properties (PEPM₂₀ and PEPM₂₃) (FIG. 5A). Therefore, the cation incorporation ratio was controlled in substituted piperidinium (Et (2), nBu (3), and nOct (4)) and quinuclidinium (5) AAEMs to be approximately 20 mol % (PEPE₂₀, PEPB₁₈, PEPO₂₃, and PEQ₂₀, respectively). Excellent WU (within 40%) and ΔL (within 11%) performance was observed for all membranes with different alkyl substituents. PEPB₁₈ and PEPO₂₃ exhibited relatively low hydroxide conductivity, but they became significantly more conductive after 1 M KOH_{aq} treatment at 80° C. for 3 days (see FIG. 7). This is likely because their hydrophobic side chains slowed down the anion exchange process, so that longer time and higher temperatures were required for thorough hydroxide exchange. The hydroxide conductivities at 80° C. of AAEMs with methyl substituents were found to be in the range of 74-95 mS/cm (FIG. 5B), which are comparable to previously reported piperidinium functionalized AAEMs with similar IECs.

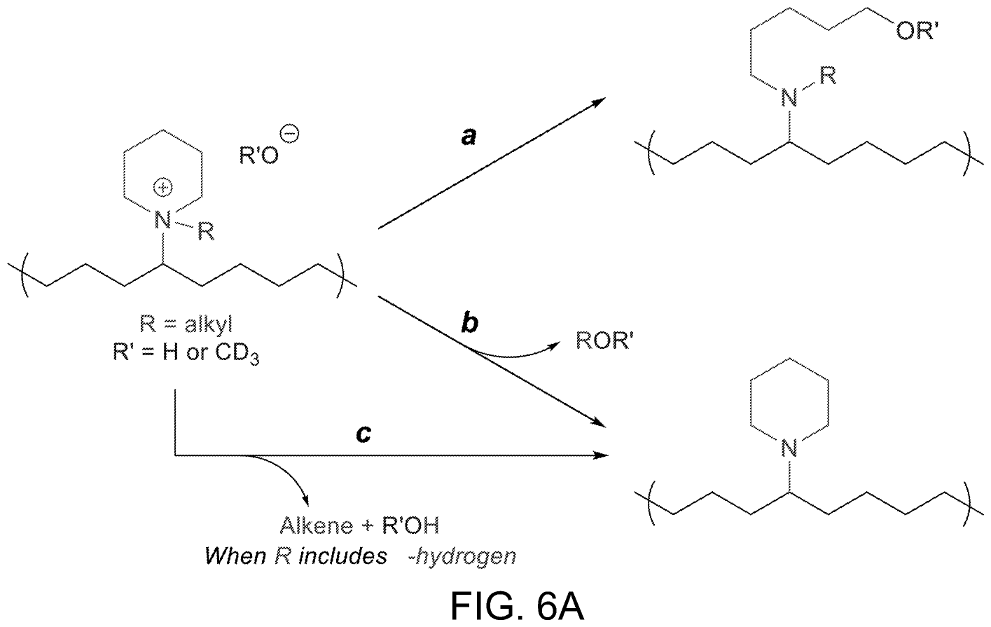

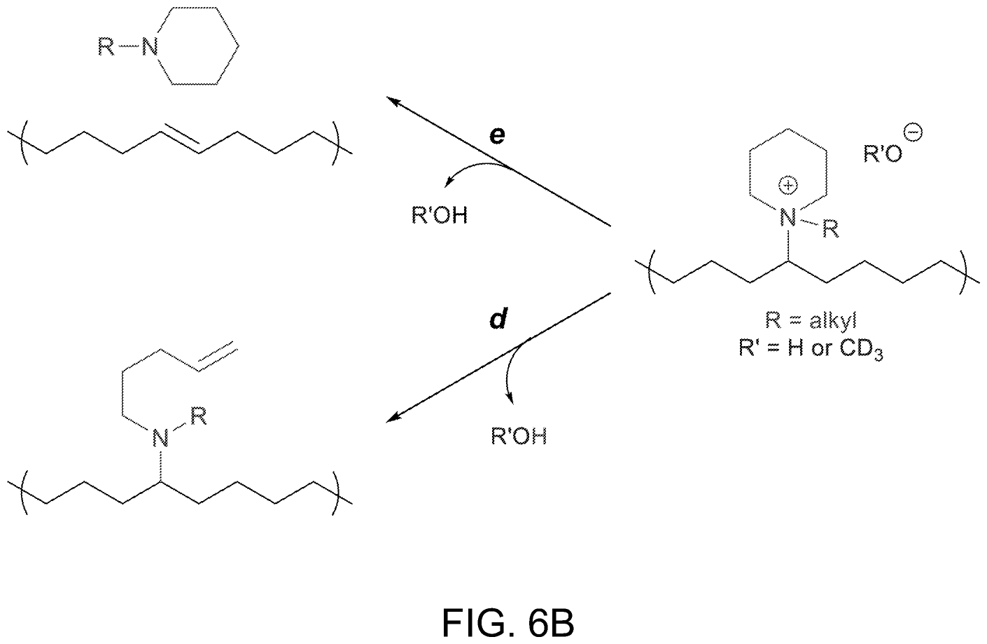

Membrane alkaline stability. In line with previously observed mechanisms for cation degradation, it was expected for there to be two major degradation mechanisms (i.e., nucleophilic substitution and Hofmann elimination) at three different sites in these piperidinium-functionalized AAEMs (i.e., the piperidinium ring, the alkyl substituent, and the PE backbone). Since nucleophilic substitution at the methine center of the polymer backbone should be relatively unlikely, it was anticipated for there to be the five possible degradation pathways summarized in FIGS. 6A-6B. Pathways a and b arise from nucleophilic substitution, while pathways c, d, and e derive from β-hydrogen Hofmann elimination.

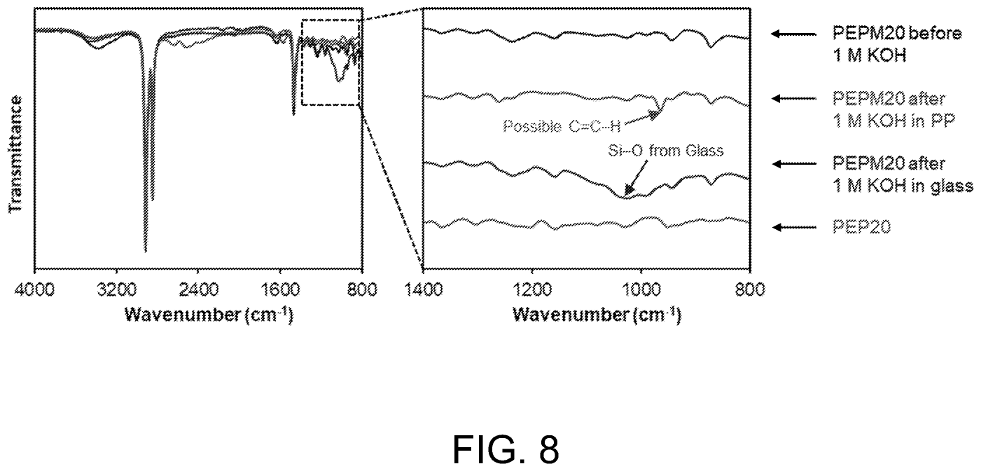

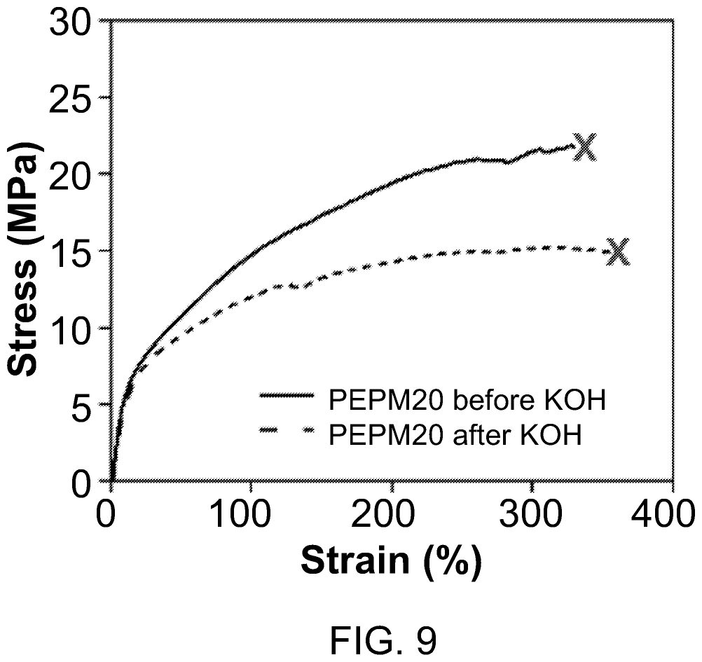

The membranes were soaked as strips in 1 M KOH_{aq} at 80° C. in polypropylene (PP) vials for 30 days, and their alkaline stabilities were evaluated by both changes in hydroxide conductivities FIG. 7) and Fourier-transform infrared (FT-IR) spectroscopy (FIG. 8). The methyl-substituted piperidinium PEPM₂₀ and quinuclidinium PEQ₂₀ showed the best conductivity stability (80% and 71% retained conductivity, respectively). The hydroxide conductivities of PEPB₁₈ and PEPO₂₃ boosted during the first three days with the plausible reasons discussed above, while drastic conductivity decreases were observed afterwards (21% and 40% remaining, respectively). AAEMs with ethyl substituents (PEPE₂₀) exhibited obvious conductivity loss from the beginning of the stability tests (42% remaining after 30 days). These overall stability trends agreed well with previous reports. One of the most stable AAEMs, PEPM₂₀, was further evaluated for its mechanical stability before and after the alkaline treatment (FIG. 9). It was found that the as-synthesized membrane PEPM₂₀ (fully hydrated in the iodide form) was strong and ductile (22 MPa stress and 330% strain at break), then the membrane became relatively weaker (15 Mpa) yet more ductile (357% strain) after soaking in 1 M KOH_{aq} at 80° C. for 30 days. Their excellent mechanical properties were comparable to Nafion™ N115 (32 Mpa stress and 310% strain at break). The elongation properties of these PE-based AAEMs significantly outperformed the rigid AAEMs derived from poly(aromatic)s.

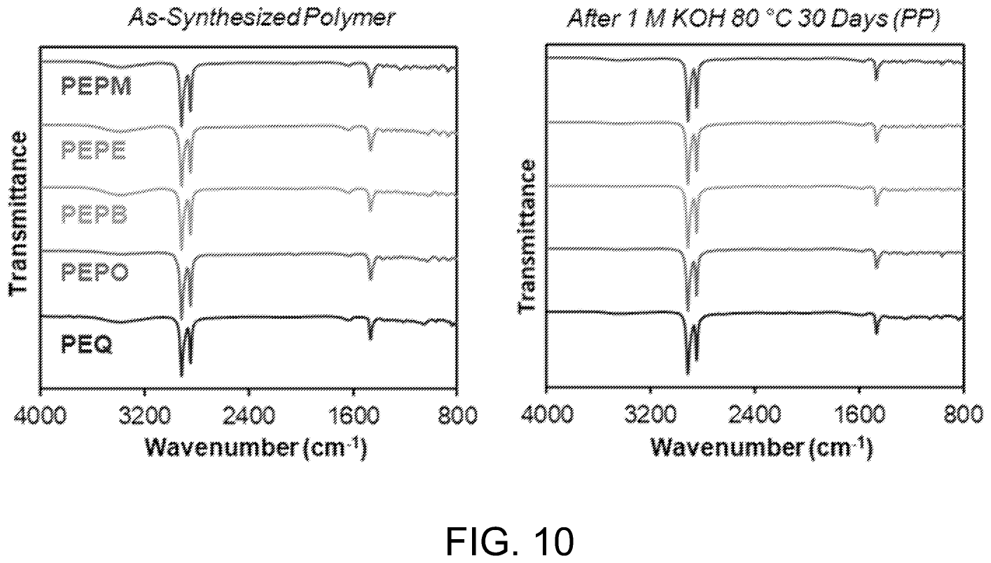

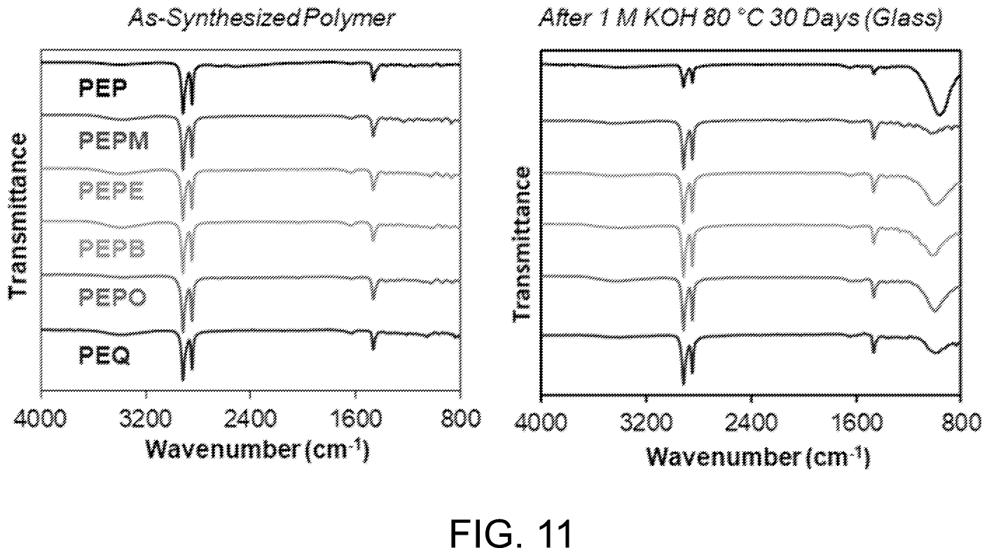

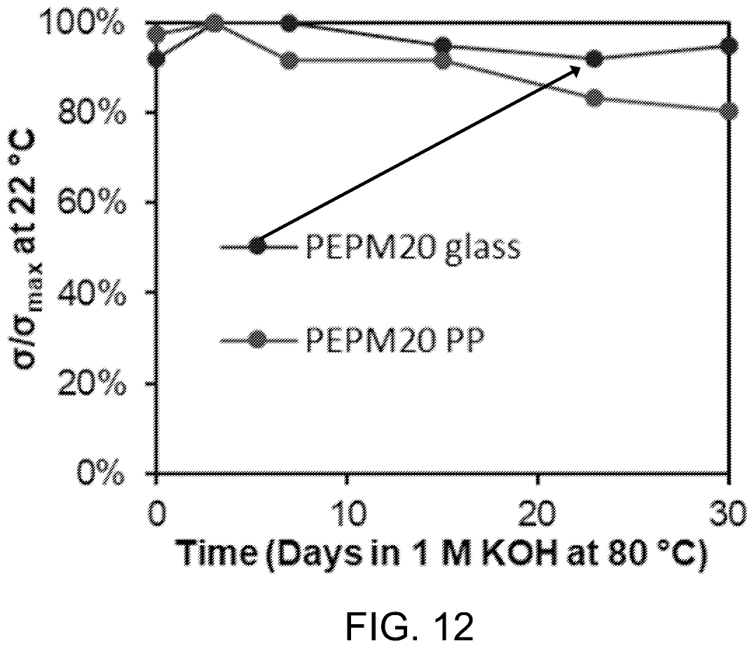

To interrogate the degradation pathways, Fourier-transform infrared (FT-IR) spectroscopy was used to study the structural changes in these membranes before and after the alkaline treatment (FIG. 8). Piperidine-functionalized PE (PEP₂₀) was also prepared from the HCl adduct of the hydroamination intermediate (compound (S1)) using an analogous ROMP copolymerization strategy followed by deprotonation to mimic some of the proposed degradation products (pathways b and c in FIG. 6A). Since the as-prepared PE-based AAEMs cannot be dissolved in common organic solvents, solid-state characterization methods such as FT-IR are advantageous. Originally, it was proposed that the piperidinium AAEMs would give extremely clear FT-IR spectra, as there are only three types of bonds in these aromatic-free polymers (i.e., C—C, C—H, and C—N bonds). However, as shown in FIGS. 8 and 10, it was noticed that the signals of C—N bonds were too weak to be confidently assigned in comparison to the strong C—H bond signals (1460 cm⁻¹ and 2800-3000 cm⁻¹). The small hump peaks at 1650 cm⁻¹ and 3300 cm⁻¹ were from water in membranes. The FT-IR spectra of PEP, PEPM, PEPE, PEPB, PEPO, and PEQ were almost the same (FIG. 10), suggesting that FT-IR spectroscopy might not be sensitive enough to distinguish piperidine and piperidinium in these polymer samples. A new peak at 964 cm⁻¹ possibly suggested the presence of alkene groups in the polymer after alkaline treatment (FIG. 8), yet it is hard to clearly assign it as pathways d or e in FIG. 6B. As an important note, it was observed that when the stability studies were performed in soda lime glass vials, there were always new broad peaks around 1050 cm⁻¹ in FT-IR spectra for all the samples after alkaline treatment (see FIGS. 8 and 11). These peaks were attributed to Si—O bonds from glass etching, as the weight of the glass vials was measured dropping by near 0.5 g after the stability studies (see Table 8). Relatively slower conductivity decrease as also observed in glass vials (FIG. 12). Although similar glass etching problems have been observed in alkaline stability studies, this is the first spectroscopic evidence to prove that the detached Si—O substances could be adsorbed by polymer samples to affect their ionic conductivities. Plastic containers made from PP and PTFE are recommended to perform aqueous alkaline stability tests.

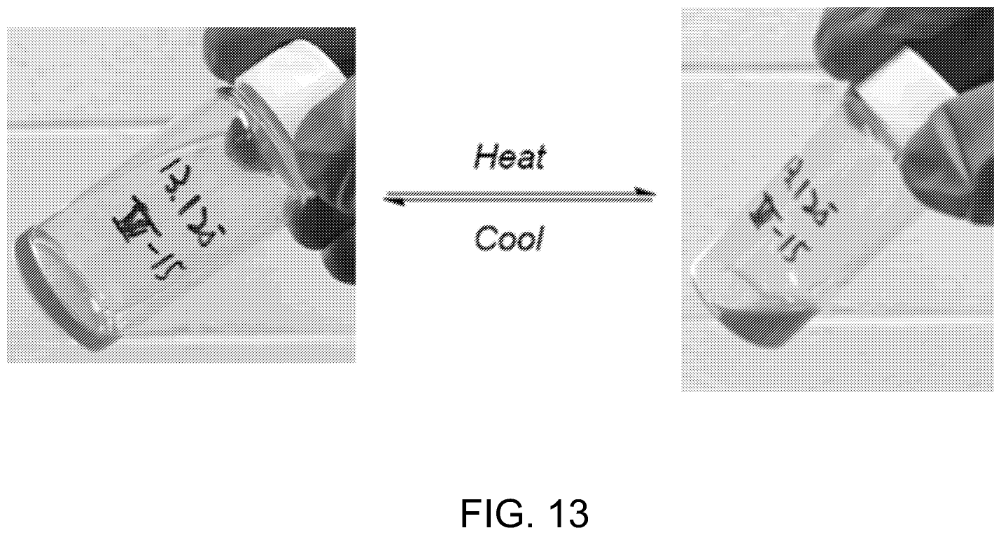

To gain more insight into the degradation mechanism, it was decided to use solution-based ¹H NMR analysis, as it can provide quantitative degradation kinetics and clearer degradation product assignments. Previously alkaline stability studies were conducted under accelerated degradation conditions by treating model compounds or polymers with KOH solutions in CD₃OH using 3-(trimethylsilyl)-1-propanesulfonic acid sodium salt as the internal standard at 80° C. in sealed NMR tubes. As a side note, control experiments showed that borosilicate glass NMR tubes were more resistant to basic corrosion and there were almost no changes to their weights after the KOH/methanol stability studies (see Table 8). One requirement of this NMR stability protocol is that the studied model compounds or polymers must be soluble in methanol. Fortunately, their solubilities can easily be tuned by changing the cation incorporation ratio and molecular weight during the ROMP copolymerization process. For example, to get the previously mentioned mechanically strong AAEMs, a mol ratio of [COE]:[piperidinium]:[Ru] of 800:200:1 was used. By changing the ratio to 200:150:1, cationic polymers were obtained that were soluble in n-propanol (5 wt. %) at 50° C., which are ideal candidates as AEI solutions (FIG. 13). By further changing the feeding ratio to 25:50:1, a series of oligomers were prepared that were soluble in methanol at 50° C. Notably, the cation incorporation percentage was slightly lower than expected in these oligomers, and monomers 1-5 gave rise to oligomers PEPM₅₂, PEPE₅₄, PEPB58, PEPO₆₄, and PEQ₅₅, respectively.

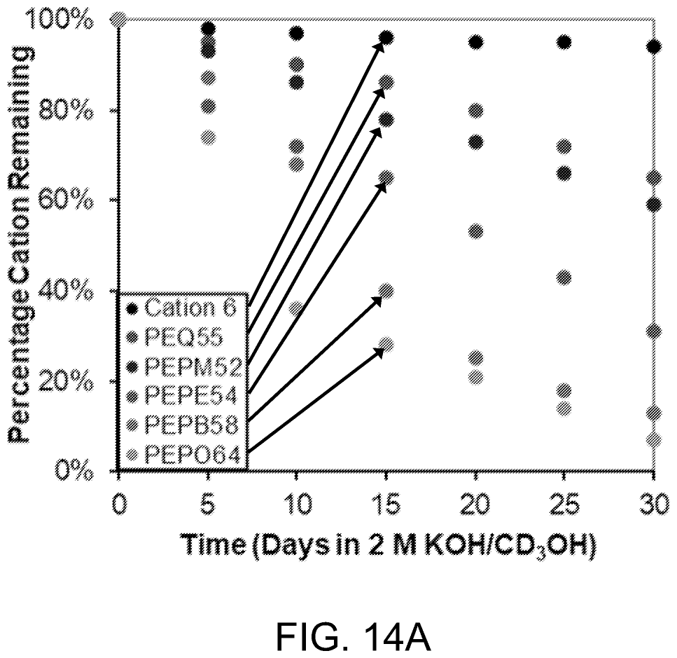

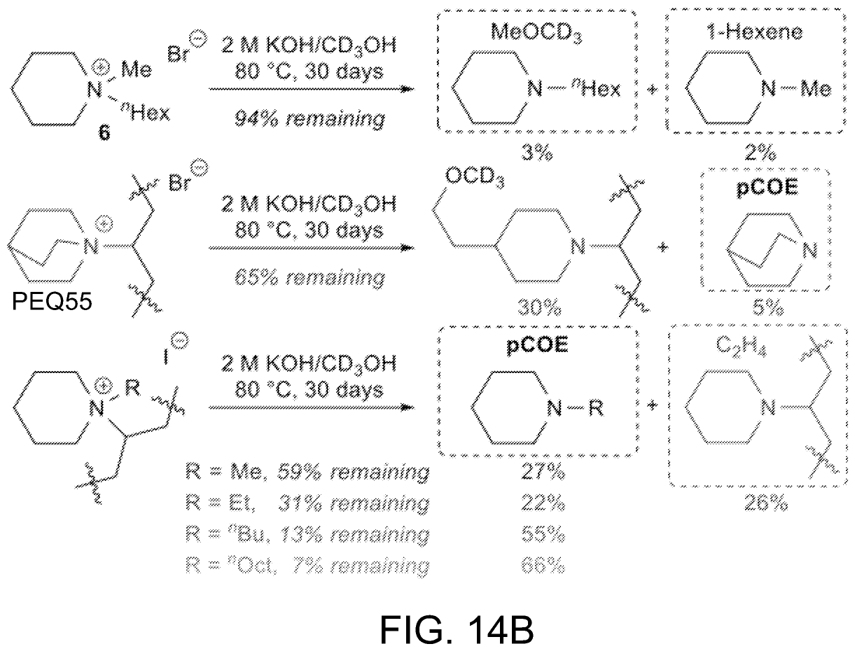

Five cationic oligomers and a small molecule model compound N-hexyl-N-methylpiperidinium bromide (6) were then subjected to the accelerated alkaline degradation conditions: 2 M KOH in CD₃OH at 80° C. for 30 days (FIG. 14). The use of CD₃OH makes anions ([OH]⁻ and [OCD₃]⁻) more reactive due to a smaller hydration sphere, and also prevents potential H/D exchange reactions. Cationic model compound 6 had very good alkaline stability (94% remaining after 30 days), and small amounts of byproducts were detected from both nucleophilic demethylation (3%) and Hofmann elimination (2%). However, much faster cation degradation was observed in oligomers with the general trend being PEQ≈PEPM>PEPE>PEPB>PEPO. Oligomers PEPM₅₂, PEPB₅₈, and PEPO₆₄ decomposed mostly through backbone Hofmann elimination I under these accelerated KOH/methanol conditions, as large quantities of N-alkylpiperidines were detected as byproducts (FIG. 14). Degradation of 27%, 55%, and 66% from N-methyl-, N-butyl-, and N-octyl-piperidine was observed compared to degradation of 41%, 87%, and 93% from the corresponding PEPM₅₂, PEPB₅₈, and PEPO₆₄, respectively. In all these cases, a broad single peak around 5.4 ppm was also detected, which is consistent with internal alkene peaks. The trend of increasing length of alkyl chains facilitating Hofmann elimination elsewhere is consistent with previous observations for piperidinium-functionalized AAEMs, although ring-opening Hofmann elimination was the major degradation pathway due to rigid substituents on the piperidinium ring. Ring-opening Hofmann elimination and nucleophilic ring opening degradation may also occur under the conditions disclosed herein, but these minor degradation pathways are extremely difficult to be accurately quantified by ¹H NMR analysis in the complicated reaction system. Interestingly, a volatile degradation byproduct, ethylene, was detected from the degradation of PEPE₅₄ oligomer (31% remaining). The backbone elimination (22%, e) and ethyl elimination (26%, c) gave similar rates under the testing conditions. Quinuclidinium PEQ₅₅ gave 30% nucleophilic ring-opening degradation out of its 35% cation degradation, probably owing to the ring strain in this bicyclic scaffold. It is noteworthy that for all these oligomer studies in sealed NMR tubes, the formation of dark insoluble materials was observed, which likely formed as the oligomers became electroneutral and less polar after degradation, diminishing their solubility in methanol. These findings highlight the importance of using an internal standard to track polymer alkaline stabilities through ¹H NMR analysis, as key information about volatile and insoluble byproducts could be easily missed otherwise.

In this work, it was demonstrated that a photocatalytic hydroamination reaction enables the facile synthesis of significant quantities of a piperidinium-cyclooctene monomer, which is quickly converted through a ROMP/hydrogenation sequence to materials for alkaline fuel cell applications. It is believed that these materials are the first aromatic-free, piperidinium- and quinuclidinium-functionalized AAEMs with PE backbones. This strategy not only expediently produced AAEMs with strong mechanical properties, high hydroxide conductivity, and good alkaline stability, but also enabled performance of systematic studies to compare different alkaline stability testing conditions (containers) and different characterization techniques. By employing a combination of FT-IR, 1H NMR, and DFT analysis, the primary degradation pathways in these AAEMs were identified, and the crucial role played by polymer backbone conformations in regulating degradation pathways based on Hofmann elimination along the PE backbone were elucidated. In doing so, it was found that methyl-substituted-piperidinium- and quinuclidinium-functionalized AAEMs (PEPM and PEQ) gave the highest alkaline stability. Due to the ease of their preparation, it is expected that these cation-functionalized PE materials will be desirable candidates for anion exchange membranes with broader applications beyond AEMFCs.

### EXAMPLE 2

The following is an example of: piperidinium-functionalized cyclooctene monomers and quinuclidinium-functionalized cyclooctene monomers of the present disclosure, and methods of making and using said monomers; piperidinium-functionalized polyethylenes (PEMs) of the present disclosure and quinuclidinium-functionalized polyethylenes (PEQs) of the present disclosure, and methods of making and using said PEMs and PEQs; and alkaline anion exchange membranes (AAEMs) of the present disclosure, methods of making and using said AAEMs.

Experimental Methods and Materials. ¹H and ¹³C NMR spectra were collected in deuterated solvents on Varian INOVA 400, Bruker 500, or Varian INOVA 600 NMR spectrometers at 22° C. or 50° C. with shifts reported relative to the residual solvent peaks (CDCl7.26 ppm (¹H) and 77.16 ppm (¹³C); or CD₃OD or CD₃OH 3.31 ppm (¹H) and 49.00 ppm (¹³C)). Data for ¹H NMR are reported as follows: chemical shift (6 ppm), broad peak (br), apparent (appr.) multiplicity (s=singlet, d=doublet, t=triplet, q=quartet, m=multiplet), coupling constant (Hz) and integration; data for ¹³C NMR are reported in terms of chemical shift and no special nomenclature is used for equivalent carbons. IR spectra were recorded on a Bruker Vertex V80v Vacuum FT-IR spectrometer using the attenuated total reflectance (ATR) mode of acquisition and are reported in terms of frequency of absorption (4000-800 cm⁻¹). High resolution mass spectrometry (HRMS) analyses were obtained at Princeton University Mass Spectrometry Facility using an Agilent 6210 TOF LC/MS (Electrospray Ionization, ESI).

All reactions and manipulations of air or water sensitive compounds were carried out under dry nitrogen using a Braun UniLab drybox or standard Schlenk techniques unless otherwise specified. Methyl iodide, triphenylphosphine, thiophenol, 2-bromo-5-methylpyridine, XPhos Pd G3, Ir(III) chloride hydrate, 4,4′-di-tert-butyl-2,2′-bipyridine^{,} and Grubbs' 2nd generation catalyst (Cl2Ru(IMes)(PCy3)CHPh) were purchased from Sigma-Aldrich and used as received. Piperidine and cis-cyclooctene (95%) were purchased from Sigma-Aldrich and distilled prior to use. 2-(Piperidin-4-yl)ethanol was purchased from J&K Scientific and used as received. Ethyl iodide, n-butyl iodine, n-octyl iodide and 3-(trimethylsilyl)-1-propanesulfonic acid sodium salt (NaDSS) were purchased from TCI and used as received. N-Bromosuccinimide and silver hexafluorophosphate were purchased from Oakwood Chemical and used as received. (2,4-Difluorophenyl)boronic acid was purchased from Accela and used as received. Crabtree's catalyst [(COD)Ir(py)(PCy3)]PF6 was purchased from Strem and used as received. Methanol-d₃ (CD₃OH) was purchased from Acros and used as received. Potassium hydroxide, sodium bicarbonate, and sodium chloride were purchased from Mallinckrodt and used as received. All solvents (methylene chloride, diethyl ether, tetrahydrofuran, acetonitrile, toluene, and methanol) were purchased from Sigma-Aldrich or Mallinckrodt. All solvents were purified according to the method of Grubbs. Hydrogen (99.99%) was purchased from Airgas. NMR solvents (CDCl₃, CD₃OD, CD₂Cl₂) were purchased from Cambridge Isotope Laboratories (CIL) and used as received. 2,4,6-Triisopropylbenzenethiol (TRIP-thiol) was prepared according to literature procedure. 1-Hexyl-1-methylpiperidin-1-ium bromide (6) was prepared according to literature procedures. Chromatographic purification of products was accomplished by flash chromatography on Silicycle F60 silica gel.

The in-plane hydroxide conductivity of the AAEM sample was measured by four-probe electrochemical impedance spectroscopy (EIS) using a Solartron 1280B electrochemical workstation along with ZPlot and ZView software. The conductivity cell was purchased from BekkTeck LLC (Loveland, CO), and a helpful schematic and description of a similar experimental setup has been reported. A strip of the thin film in iodide form (ca. 4 cm long×0.5 cm wide) was converted to the hydroxide form by immersing it in a stirring 30 mL portion of 1 M potassium hydroxide for a minimum of 2 h and the 1 M KOH solution was replaced twice with fresh solution during that time. Residual potassium hydroxide was washed away by immersing the membrane in 3×60 mL portions of deionized water for 20 minutes each. The AAEM was then clamped into the cell using a Proto 6104 torque screwdriver set to 1 inch ounce and completely immersed in deionized water at 22° C. during the measurement time. EIS was performed by imposing a small sinusoidal (AC signal) voltage, 10 mV, across the membrane sample at frequencies between 20,000 Hz and 0.1 Hz (scanning from high to low frequencies) and measuring the resultant current response. A Bode plot was used to assess the frequency range over which the impedance approached a constant and the phase angle approached zero. In a Nyquist plot of the data, the high frequency intercept on the real impedance axis was taken to be the resistance of the membrane. This was then used to calculate the hydroxide conductivity by employing the following formula: σ=L/Z′×A where L is the length between sense electrodes (0.425 cm), Z′ is the real impedance response at high frequency, and A is the membrane area available for hydroxide conduction (width×thickness). The dimensional measurements were performed using a digital micrometer (±0.001 mm) purchased from Marathon Watch Company Ltd. (Richmond Hill, ON). The hydroxide conductivity was measured for a minimum of three separate AAEMs (per composition)

Water uptake and percentage dimensional change were measured by the change between the fully hydrated and dried AAEMs. Conversion to the hydroxide form was achieved by immersing it in a stirring 30 mL portion of 1 M potassium hydroxide for a minimum of 2 h and the 1 M KOH solution was replaced twice with fresh solution during that time. Residual potassium hydroxide was washed away by immersing the membrane in 3×60 mL portions of deionized water for 20 minutes each. Immediately following hydroxide ion exchange, a sample was dried with a paper towel, measured by length, and weighed on the balance with a piece a weighing paper. The thin film (in the hydroxide form) was dried under full vacuum at 60° C. in order to completely dehydrate it and then weighed and measured. The water uptake percentage value was calculated by: WU=[(Mass_{wet}−Mass_{dry})/Mass_{dry}]×100. The dimensional change percentage was calculated b—: ΔL=[(L_{wet}−L_{dry})/L_{dry}]×100.

The samples were exchanged to the iodide form in 2 M KI (aq) for 48 hours and washed with water before the mechanical and FT-IR test. Uniaxial tensile elongation was carried out using a Shimadzu Autograph AGS-X tensile tester. The conductive stability was evaluated according to literature procedures. 5 Strips of the thin film in the iodide form (ca. 3 cm long×0.3 cm wide) were removed from 1 M KOH following the typical exchange procedure and placed in a polypropylene (PP) or a glass vial containing 1 M KOH. The vial was sealed in air and was heated at 80° C. The KOH solution was periodically replaced with fresh solution to ensure the alkaline concentration remained unchanged. At specified time intervals, membrane strips were re-exchanged with 1 M KOH (typical procedure with a 2 h exchange), washed with water to remove any residual base, and the in-plane hydroxide conductivity was measured at 22° C.

Synthesis of [Ir(dF(Me)ppy)₂(dtbbpy)]PF₆. Note: This photocatalyst is commercially available from both Sigma-Aldrich and Strem Chemicals

### 2-(2,4-difluorophenyl)-5-methylpyridine

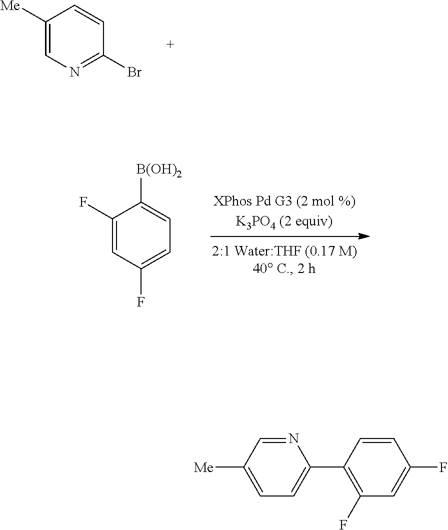 [SMILES: Cc1ccc(Br)nc1 ; OB(O)c1ccc(F)cc1F ; Cc1ccc(-c2ccc(F)cc2F)nc1]

To a 50 mL round-bottom flask was added K₃PO₄ (2.30 g, 10 mmol, 2.0 equiv) and water (20 mL). This solution was degassed by sparging with N₂ for 20 minutes. To a 100 mL round-bottom flask charged with a magnetic stir bar was added 2-bromo-5-methylpyridine (0.86 g, 5.0 mmol, 1.0 equiv), (2,4-difluorophenyl)boronic acid (1.18 g, 7.5 mmol, 1.5 equiv), and XPhos Pd G3 (85 mg, 0.10 mmol, 2 mol %). The flask was evacuated and backfilled with N₂ three times. To this flask was added THF (10 mL) and the aqueous K₃PO₄ solution. The reaction was heated to 40° C. with vigorous stirring for 2 h. The reaction mixture was then cooled to room temperature and diluted with water (30 mL) and Et₂O (30 mL). The aqueous phase was separated in a separatory funnel and washed with Et₂O (20 mL) three times. The combined organic layers were washed with brine (30 mL), and then dried over Na₂SO₄. Following removal of the solvent in vacuo, the crude residue was purified by silica gel chromatography (gradient from 0-5% EtOAc/hexane) to afford 2-(2,4-difluorophenyl)-5-methylpyridine (0.44 g) as a pale yellow solid in 43% yield. Characterization data was consistent with reported literature values.

### [Ir(dF(Me)ppy)₂Cl]₂-dimer

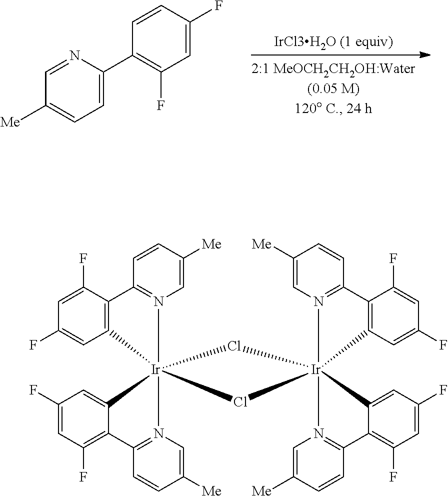

To a flame-dried 250 mL three-necked round-bottom flask with a reflux condenser and stir bar was added IrCl₃·H₂O (0.32 g, 1.0 mmol, 1.0 equiv) and 2-(2,4-difluorophenyl)-5-methylpyridine (0.42 g, 2.1 mmol, 2.1 equiv). The flask was evacuated and backfilled with N₂ three times. 2-Methoxyethanol (13 mL) and water (6.7 mL), each degassed by sparging N₂ for 20 minutes, were added and the reaction was heated to 120° C. overnight. The reaction mixture was cooled to room temperature, which resulted in the formation of a large amount of yellow precipitate. The solid was filtered and washed with water (20 mL) three times to afford 0.49 g of the crude dimer in 77% yield. The crude dimer was carried on without any further purification.

### [Ir(dF(Me)ppy)₂(MeCN)₂]PF₆

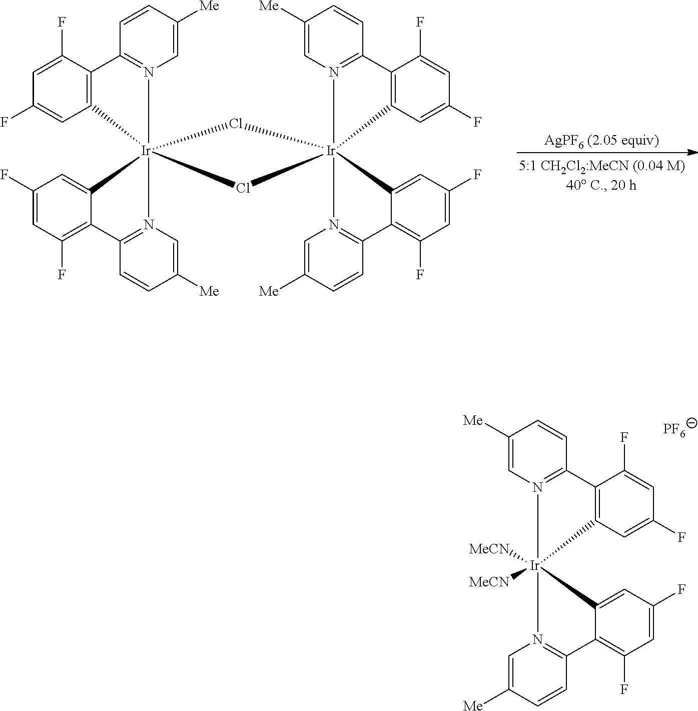

To a 50 mL three-necked round-bottom flask charged with a stir bar was added the crude [Ir(dF(Me)ppy)₂Cl]₂-dimer (0.48 g, 0.38 mmol, 1.0 equiv) from the previous step. The flask was pumped into a glovebox wherein AgPF₆ (0.20 g, 0.78 mmol, 2.1 equiv) was added and the flask capped. After exchanging a cap for a reflux condenser, the flask was evacuated and backfilled with N₂ three times. Dry CH₂Cl₂ (8.0 mL) and MeCN (1.6 mL) were added and the reaction mixture was heated to 40° C. for 20 h. The reaction mixture was cooled to room temperature and solvent removed in vacuo. After taking up the crude residue in acetone, the AgCl salts were filtered off. Pentane was added to the filtrate to afford 0.48 g of [Ir(dF(CF₃)ppy)₂(MeCN)₂]PF₆ as a yellow solid in 85% yield. The crude product was carried on without any further purification.

### [Ir(dF(Me)ppy)₂(dtbbpy)]PF₆

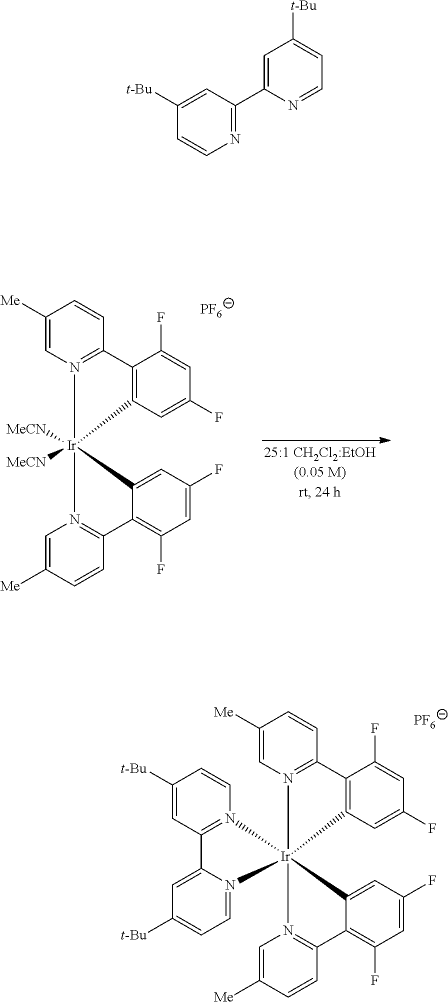

To a 100 mL round-bottom flask with a stir bar was added [Ir(dF(Me)ppy)₂(MeCN)₂]PF₆ (0.48 g, 0.65 mmol, 1.0 equiv) and 4,4′-di'tert-butyl-2,2′-bipyridine (0.21 g, 0.77 mmol, 1.2 equiv). CH₂Cl₂ (12 mL) and EtOH (0.5 mL) were added and the reaction mixture was stirred at rt overnight. The reaction mixture was filtered through a pad of celite and washed copiously with CH₂Cl₂ to dissolve as much of the crude iridium complex as possible. The resulting filtrate was concentrated in vacuo and recrystallized from acetone and pentane to afford 0.54 g of [Ir(dF(Me)ppy)₂(dtbbpy)]PF₆ as yellow crystals in 82% yield. Characterization data was consistent with reported literature values.

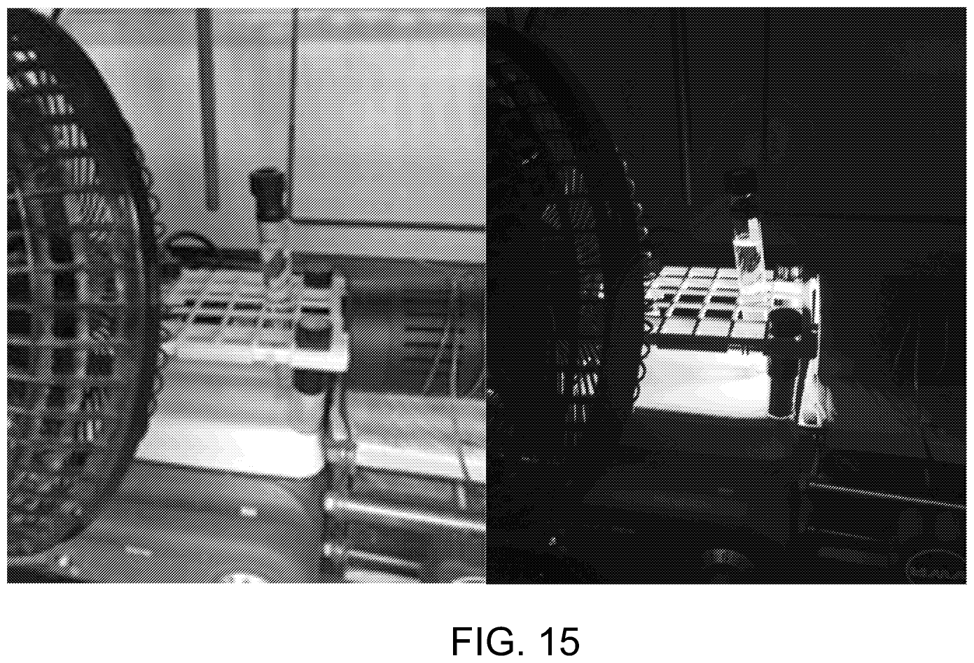

Synthesis of Piperidinium Monomers. General Procedure: An oven-dried 16×25 mm screw-capped culture tube was equipped with an oven-dried stir bar and charged with [Ir(dF(Me)ppy)₂(dtbbpy)]PF₆ (5.1 mg, 0.0050 mmol, 0.25 mol %). The reaction was then pumped into the glovebox, wherein it was sealed with a Teflon cap. Electrical tape was used to seal the sides of the cap, and the reaction vessel was removed from the glovebox. Degassed anhydrous toluene (10 mL, 0.20 M) was then added via syringe, followed by 1,5-cyclooctadiene (2.5 mL, 20 mmol, 10 equiv), piperidine (200 μL, 2.0 mmol, 1.0 equiv), and thiophenol (31 μL, 0.30 mmol, 15 mol %). The top of the culture tube was then wrapped in parafilm. This pale yellow solution was irradiated by a single 34 W Kessil KSH₁₅₀B blue LED lamp and magnetically stirred for 24 h (FIG. 15). A small rotary fan was placed adjacent to the vial to cool the reaction during irradiation. A typical reaction was measured to run at about 35° C. Once completed, the reaction mixture was transferred to a tared 25 mL round-bottom flask and concentrated via rotary evaporation. The flask was then evacuated, backfilled with N₂, and quaternized with alkyl iodides.

(Z)-1-(Cyclooct-4-en-1-yl)-1-methylpiperidinium iodide (1). Following the general procedure, degassed anhydrous tetrahydrofuran (10 mL, 0.4 M) and iodomethane (0.25 mL, 4.0 mmol, 2.0 equiv) were added into the flask via syringe. This mixture was stirred at 65° C. for 2 h. Over the course of the reaction, white solids crashed out of solution. After cooling the reaction to 22° C., the solids were triturated. This process was repeated twice more with 5 mL diethyl ether, then the residual solvent was removed under reduced pressure to reveal the title compound as a white solid (0.54 g, 81% yield). ¹H NMR (500 MHz, CDCl₃): δ 5.83 (appr. q, J=7.9 Hz, 1H), 5.65 (appr. q, J=9.2 Hz, 1H), 3.98-3.83 (m, 2H), 3.83-3.74 (m, 1H), 3.67-3.74 (m, 2H), 3.16 (s, 3H), 2.58-2.43 (m, 1H), 2.35-2.18 (m, 3H), 2.07 (appr. t, J=10.7 Hz, 1H), 2.02-1.69 (m, 10H), 1.46-1.30 (m, 1H); ¹³C NMR (125 MHz, CDCl₃): δ 131.8, 128.4, 59.9, 59.4, 44.8, 28.2, 26.6, 26.3, 25.9, 22.6, 20.7, 20.5, 20.3; HRMS (ESI+): Calculated for C₁₄H₂₆N ([M−I]⁺): 208.2065, Found 208.2068.

(Z)-1-(Cyclooct-4-en-1-yl)-1-ethylpiperidin-1-ium iodide (2). Following the general procedure, degassed anhydrous acetonitrile (5.0 mL, 0.4 M) and iodoethane (0.80 mL, 10 mmol, 5.0 equiv) were added into the flask via syringe. This mixture was stirred at 80° C. for 20 h. After this time, the reaction was cooled to 22° C. and most of the solvent (ca. 80%) removed via rotary evaporation. To this viscous yellow oil was added 5 mL diethyl ether, resulting in the rapid formation of pale yellow precipitates. This was stirred for 5 min, at which time the stirring was terminated and the solids were triturated. This process was repeated twice more, then the residual solvent was removed under reduced pressure to reveal the title compound as a pale yellow solid (0.55 g, 79% yield). ¹H NMR (500 MHz, CDCl₃): δ 5.85 (appr. q, J=8.0 Hz, 1H), 5.64 (appr. q, J=9.8 Hz, 1H), 3.74 (dq, J=13.9, 6.5 Hz, 1H), 3.69-3.54 (m, 5H), 3.52-3.44 (m, 1H), 2.57-2.38 (m, 2H), 2.35-2.19 (m, 3H), 2.09-1.75 (m, 11H), 1.37 (t, J=6.9 Hz, 3H); ¹³C NMR (125 MHz, CDCl₃): δ 132.0, 128.1, 55.3, 28.5, 26.7, 26.6, 26.0, 22.7, 20.8, 20.0, 19.9, 8.6; HRMS (ESI+): Calculated for C₁₅H₂₈N ([M−I]⁺): 222.2222, Found 222.2221.

(Z)-1-Butyl-1-(cyclooct-4-en-1-yl)piperidin-1-ium iodide (3). Following the general procedure, degassed anhydrous acetonitrile (5.0 mL, 0.4 M) and iodobutane (1.8 mL, 10 mmol, 5.0 equiv) were added into the flask via syringe. This mixture was stirred at 80° C. for 24 h. After this time, the reaction was cooled to 22° C. and most of the solvent (ca. 80%) removed via rotary evaporation. To this viscous yellow oil was added 5 mL diethyl ether, resulting in the rapid formation of pale-yellow precipitates. This was stirred for 5 min, at which time the stirring was terminated and the solids triturated. This process was repeated twice more, then the residual solvent was removed under reduced pressure to reveal the title compound as a pale yellow solid (0.60 g, 79% yield). ¹H NMR (500 MHz, CDCl₃): δ ¹H NMR (500 MHz, CDCl₃): δ 5.84 (dt, J=10.5, 7.9 Hz, 1H), 5.64 (td, J=10.1, 6.8 Hz, 1H), 3.72-3.57 (m, 4H), 3.52-3.46 (m, 2H), 3.43-3.37 (m, 1H), 2.58-2.43 (m, 2H), 2.36-2.19 (m, 3H), 2.06-1.78 (m, 11H), 1.74-1.66 (m, 1H), 1.46 (q, J=7.4 Hz, 2H), 1.34 (dddd, J=14.0, 10.6, 10.2, 4.7 Hz, 1H), 1.01 (t, J=7.4 Hz, 3H); ¹³C NMR (125 MHz, CDCl₃): δ 132.0, 128.1, 66.0, 55.9, 29.4, 28.6, 26.8, 26.7, 25.9, 25.2, 24.6, 22.7, 20.7, 20.3, 20.1, 20.0, 14.0; HRMS (ESI+): Calculated for C₁₇H₃₂N ([M−I]⁺): 250.2535, Found 250.2534.

(Z)-1-(Cyclooct-4-en-1-yl)-1-octylpiperidin-1-ium iodide (4). Following the general procedure, degassed anhydrous acetonitrile (5.0 mL, 0.4 M) and iodooctane (1.8 mL, 10 mmol, 5.0 equiv) were added into the flask via syringe. This mixture was stirred at 80° C. for 24 h. After this time, the reaction was cooled to 22° C. and most of the solvent (ca. 80%) removed via rotary evaporation. To this viscous yellow oil was added 5 mL diethyl ether, resulting in the rapid formation of pale yellow precipitates. This was stirred for 5 min, at which time the stirring was terminated and the solids triturated. This process was repeated twice more, then the residual solvent was removed under reduced pressure to reveal the title compound as a pale yellow solid (0.66 g, 76% yield). ¹H NMR (500 MHz, CDCl₃): δ 5.85 (appr. q, J=7.9 Hz, 1H), 5.64 (appr. q, J=9.7 Hz, 1H), 3.73-3.57 (m, 4H), 3.56-3.43 (m, 2H), 3.43-3.33 (m, 1H), 2.57-2.43 (m, 2H), 2.33-2.20 (m, 3H), 2.04-1.78 (m, 11H), 1.72-1.65 (m, 2H), 1.46-1.22 (m, 12H), 0.88 (t, J=6.3 Hz, 3H); ¹³C NMR (125 MHz, CDCl₃): δ 132.1, 128.1, 55.8, 31.8, 29.3, 29.2, 28.6, 26.9, 26.8, 26.7, 25.9, 22.7, 22.7, 20.7, 20.1, 20.0, 14.2; HRMS (ESI+): Calculated for C₂₁H₄₀N ([M−I]⁺): 306.3161, Found 306.3164.

(Z)-1-(Cyclooct-4-en-1-yl)quinuclidin-1-ium bromide (5). Following the general procedure, 2-(piperidin-4-yl)ethanol (0.26 g, 2.0 mmol, 1 equiv) was used instead of piperidine. Degassed anhydrous CH₂Cl₂ (5.0 mL, 0.4 M) and triphenylphosphine (0.84 g, 3.2 mmol, 1.6 equiv) were added to the residue, then the reaction was cooled to 0° C. N-Bromosuccinimide (0.57 g, 3.2 mmol, 1.6 equiv) was added portion wise over 5 min. The reaction was allowed to slowly warm to 22° C. and stirred for 2 h. At this time, the crude reaction mixture was directly subjected to silica gel chromatography, eluting with a gradient of 100% CH₂Cl₂ to 5% MeOH in CH₂Cl₂. The clean fractions were combined and the solvent removed via rotary evaporation. It was found that upon standing for 24 h at 22° C. that the cyclization spontaneously occurred, furnishing the title compound as an off white solid (0.18 g, 30% yield). ¹H NMR (500 MHz, CDCl₃): δ 5.76 (appr. q, J=8.2 Hz, 1H), 5.62 (appr. q, J=9.9 Hz, 1H), 3.63 (t, J=7.5 Hz, 6H), 3.46-3.38 (m, 1H), 2.56-2.39 (m, 2H), 2.34-2.16 (m, 5H), 2.14-2.05 (m, 6H), 1.89-1.82 (m, 1H), 1.77-1.69 (m, 2H), 1.45-1.33 (m, 1H); ¹³C NMR (125 MHz, CDCl₃): δ 131.5, 128.6, 74.7, 52.3, 28.3, 27.6, 26.3, 25.7, 24.2, 22.8, 20.0; HRMS (ESI+): Calculated for C₁₅H₂₆N ([M−Br]⁺): 220.2065, Found 220.2070.

(Z)-1-(Cyclooct-4-en-1-yl)piperidin-1-ium chloride (S1). Following the general hydroamination procedure, the solvent was removed under reduced pressure. The crude residue was then dissolved in diethyl ether, then 1 M HCl in diethyl ether (2 equiv) was added. This was stirred for 30 minutes, over which time brown solids precipitated. These solids were then filtered. The crude brown solid (500 mg) was dissolved in 1 mL MeOH and carefully precipitated in 10 mL Et₂O. The mixture was stirred for 5 min, at which time the stirring was terminated and the solids were triturated. This process was repeated twice more, then the residual solvent was removed under reduced pressure to reveal the title compound as an off white solid (411 mg). ¹H NMR (500 MHz, CDCl₃): δ 11.64 (br s, 1H), 5.80-5.56 (m, 2H), 3.34-3.26 (m, 2H), 3.21 (ddt, J=12.1, 6.0, 2.5 Hz, 1H), 2.72 (tdt, J=12.8, 9.6, 3.4 Hz, 2H), 2.50-2.34 (m, 3H), 2.29-2.12 (m, 5H), 1.91 (dt, J=13.4, 3.6 Hz, 1H), 1.85-1.73 (m, 3H), 1.65-1.54 (m, 3H), 1.33 (appr. qt, J=13.3, 3.8 Hz, 1H); ¹³C NMR (125 MHz, CDCl₃): δ 131.1, 128.7, 66.5, 49.6, 49.1, 29.1, 28.1, 26.1, 25.3, 22.8, 22.8, 22.7, 22.6.

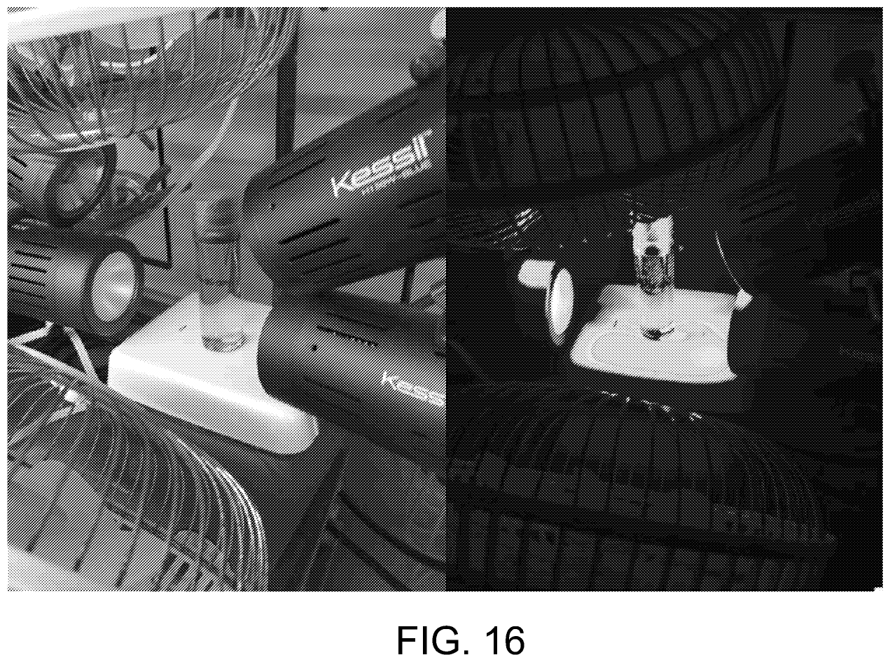

Scale-up Procedure: An oven-dried 40 mL scintillation vial was equipped with an oven-dried stir bar and charged with [Ir(dF(Me)ppy)₂(dtbbpy)]PF₆ (15 mg, 0.015 mmol, 0.25 mol %). The reaction was then pumped into the glovebox, wherein it was sealed with a Teflon cap. Electrical tape was used to seal the sides of the cap, and the reaction vessel was removed from the glovebox. Degassed anhydrous toluene (30 mL, 0.20 M) was then added via syringe, followed by 1,5-cyclooctadiene (7.4 mL, 60 mmol, 10 equiv), piperidine (593 μL, 6.0 mmol, 1.0 equiv), and thiophenol (92 μL, 0.90 mmol, 15 mol %). The top of the culture tube was then wrapped in parafilm. This pale yellow solution was irradiated by four 34 W Kessil KSH₁₅₀B blue LED lamps and magnetically stirred for 36 h (FIG. 16). Two small rotary fans were placed adjacent to the vial to cool the reaction during irradiation. Once completed, the reaction mixture was transferred to a tared 100 mL round-bottom flask and concentrated via rotary evaporation. The flask was equipped with a reflux condenser, then degassed anhydrous tetrahydrofuran (30 mL, 0.20 M) and iodomethane (0.75 mL, 12 mmol, 2.0 equiv) were added via syringe. This mixture was stirred at 65° C. for 2 h. Over the course of the reaction, white solids crashed out of solution. After cooling the reaction to 22° C., the solids were triturated. This process was repeated twice more with 15 mL diethyl ether, then the residual solvent was removed under reduced pressure to reveal (Z)-1-(Cyclooct-4-en-1-yl)-1-methylpiperidinium iodide (1) as a white solid (1.62 g, 80% yield).

### Synthesis of Piperidinium-Functionalized AAEMs

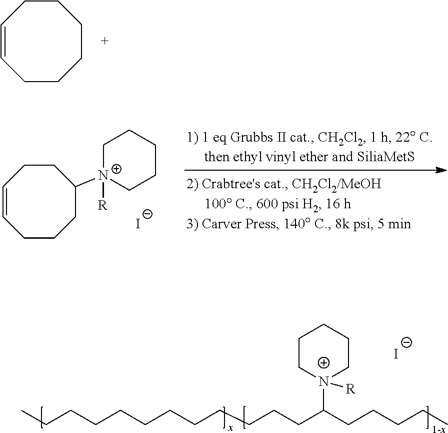

Representative Membrane Preparation Procedure: Under a nitrogen atmosphere piperidinium monomer (1) (0.20 mmol) and COE (0.80 mmol) were combined and dissolved in CH₂Cl₂ (0.80 mL). To the reaction mixture, Grubbs 2^{nd} generation catalyst (0.0010 mmol) dissolved in 0.20 mL of CH₂Cl₂ was added and the solution was stirred vigorously. The reaction was conducted for a minimum of 17 hours. The reaction mixture was then quenched with excess ethyl vinyl ether and silica-bound metal scavenger (SiliaMetS, dimercaptotriazine (DMT)), filtered, and concentrated under reduced pressure. The resultant mixture was washed with acetone three times and dried under high vacuum to afford a pale white polymer. The unsaturated copolymer was then dissolved in a 4:1 CH₂Cl₂/methanol cosolvent (10 mL) forming a yellow solution. The solution and Crabtree's catalyst (0.0020 mmol) were combined in a Parr reactor and sealed. It was pressurized to 40 atm hydrogen and then vented down to 3 atm. This process was repeated twice more to purge the reactor of air, then pressurized to 40 atm and heated to 100° C. with stirring. After 17 hours, it was cooled, vented and the slurry polymer mixture was dried under vacuum furnishing pale yellow solid. The membranes were prepared in a Carver-Press (4120 Hydraulic Unit Carver press and stainless steel die molds, Teflon protective sheets from American Durafilm, 140° C. with 8,000 psig pressure for 5 min).

Representative Ionomer Preparation Procedure: Under a nitrogen atmosphere piperidinium monomer (1) (0.15 mmol) and COE (0.20 mmol) were combined and dissolved in CH₂Cl₂ (0.80 mL). To the reaction mixture, Grubbs 2^{nd} generation catalyst (0.0010 mmol) dissolved in 0.20 mL of CH₂Cl₂ was added and the solution was stirred vigorously. The reaction was conducted for a minimum of 17 hours. The reaction mixture was then quenched with excess ethyl vinyl ether and silica-bound metal scavenger (SiliaMetS, dimercaptotriazine (DMT)), filtered, and concentrated under reduced pressure. The resultant mixture was washed with acetone three times and dried under high vacuum to afford a pale brown polymer. The unsaturated copolymer was then dissolved in a 1:1 CH₂Cl₂/methanol cosolvent (10 mL) forming a yellow solution. The solution and Crabtree's catalyst (0.0010 mmol) were combined in a Parr reactor and sealed. It was pressurized to 600 psig hydrogen and then vented down to 50 psig. This process was repeated twice more to purge the reactor of air, then pressurized to 600 psig and heated to 100° C. with stirring. After 17 hours, it was cooled, vented, and directly transferred into a glass solvent evaporation dish. The CH₂Cl₂/methanol cosolvent was removed at 50° C. for 6 hours and 70° C. for 2 hours. The resultant membrane was then soaked in 1 M Na₂CO₃ (aq) for 24 hours and washed with water to exchange the anions to carbonate. The residue polymer was then dried and dissolved in nPrOH as a 5 wt % solution. The polymer was a gel at 22° C., and it turned into solution above 50° C. (FIG. 13).

Representative Oligomer Preparation Procedure: Under a nitrogen atmosphere piperidinium monomer (1) (0.10 mmol) and COE (0.050 mmol) were combined and dissolved in CH₂Cl₂ (0.20 mL). To the reaction mixture, Grubbs 2^{nd} generation catalyst (0.0020 mmol) dissolved in 0.10 mL of CH₂Cl₂ was added and the solution was stirred vigorously. The reaction was conducted for a minimum of 17 hours. The reaction mixture was then quenched with excess ethyl vinyl ether and silica-bound metal scavenger (SiliaMetS, dimercaptotriazine (DMT)), filtered, and concentrated under reduced pressure. The resultant mixture was washed with acetone:Et₂O (1:1 v/v) three times and dried under high vacuum to afford a pale brown polymer.

The unsaturated copolymer was then dissolved in a 1:1 CH₂Cl₂/methanol cosolvent (6 mL) forming a yellow solution. The solution and Crabtree's catalyst (0.0010 mmol) were combined in a Parr reactor and sealed. It was pressurized to 40 atm hydrogen and then vented down to 3 atm. This process was repeated twice more to purge the reactor of air, then pressurized to 40 atm and heated to 100° C. with stirring. After 17 hours, it was cooled, vented, and concentrated to obtain pale brown, viscous oligomers.

The unsaturated copolymers were subjected to ¹H and ¹³C NMR analyses in CD₃OD at 50° C. before and after hydrogenation. Oligomer PEPM₅₂: before hydrogenation ¹H liNMR (500 MHz, CD₃OD): δ 5.88-5.21 (br m, 4 H), 3.72-3.37 (br m, 5 H), 3.01 (br s, 3 H), 2.40-1.49 (br m, 20 H), 1.47-1.24 (br m, 6 H); after hydrogenation ¹H NMR (500 MHz, CD₃OD): δ 3.62-3.32 (br m, 5 H), 2.97 (br s, 3 H), 2.10-1.19 (br m, 34 H); ¹³C NMR (125 MHz, CD₃OD): δ 76.8, 60.6, 43.0, 30.8, 30.5, 30.2, 29.2, 22.1, 21.3. Oligomer PEPE₅₄: before hydrogenation δ 5.85-5.30 (br m, 4 H), 3.70-3.33 (br m, 7 H), 2.44-1.49 (br m, 21 H), 1.47-1.21 (br m, 10 H); after hydrogenation ¹H NMR (500 MHz, CD₃OD): δ 3.62-3.32 (br m, 7 H), 2.15-1.57 (br m, 12 H), 1.57-1.19 (br m, 28 H); after hydrogenation ¹³C NMR (125 MHz, CD₃OD): δ 72.2, 56.0, 51.3, 30.8, 30.6, 29.4, 22.0, 20.7, 8.2. Oligomer PEPB₅₈: before hydrogenation ¹H NMR (500 MHz, CD₃OD): δ 5.90-5.24 (br m, 3 H), 3.63-3.34 (br m, 7 H), 2.37-1.25 (br m, 30 H), 1.04 (t, J=7.3 Hz, 3H); after hydrogenation ¹H NMR (500 MHz, CD₃OD): δ 3.51-3.32 (br m, 7 H), 2.13-1.57 (br m, 13 H), 1.57-1.21 (br m, 24 H), 1.04 (t, J=7.4 Hz, 1H). Oligomer PEPO₆₄: before hydrogenation ¹H NMR (500 MHz, CD₃OD): δ 6.03-5.29 (br m, 3 H), 3.67-3.32 (br m, 7 H), 2.32-1.83 (br m, 13 H), 1.83-1.51 (br m, 8H), 1.49-1.24 (br m, 15H), 0.95-0.88 (br m, 3H); after hydrogenation ¹H NMR (500 MHz, CD₃OD): δ 3.53-3.32 (br m, 7 H), 2.16-1.59 (br m, 15 H), 1.57-1.24 (br m, 28 H), 0.95-0.88 (br m, 3H). Oligomer PEQ₅₅: before hydrogenation ¹H NMR (500 MHz, CD₃OD): δ 5.79-5.27 (br m, 4 H), 3.75-3.35 (br m, 6 H), 3.28-2.90 (br m, 1 H), 2.30-1.84 (br m, 17 H), 1.77-1.24 (br m, 11H); after hydrogenation ¹H NMR (500 MHz, CD₃OD): δ 3.61-3.37 (br m, 6 H), 3.15-2.88 (br m, 1H), 2.20-2.12 (br m, 9 H), 1.72-1.19 (br m, 22 H).

The oligomers were further subjected to ¹H NMR alkaline stability studies in 2 M KOH/CD₃OH at 80° C. for 30 days and the ¹H NMR spectra was obtained at 50° C. (as described herein, Table 4, FIGS. 14A-14B).

Synthesis of Piperidine-Functionalized Polyethylene (PEP): Under a nitrogen atmosphere piperidine-HCl monomer (S1) (0.20 mmol) and COE (0.80 mmol) were combined and dissolved in CH₂Cl₂ (0.80 mL) and MeOH (0.10 mL). To the reaction mixture, Grubbs 2^{nd} generation catalyst (0.0010 mmol) dissolved in 0.20 mL of CH₂Cl₂ was added and the solution was stirred vigorously. The reaction was conducted for a minimum of 17 hours. The reaction mixture was then quenched with excess ethyl vinyl ether and silica-bound metal scavenger (SiliaMetS, dimercaptotriazine (DMT)), filtered, and concentrated under reduced pressure. The resultant mixture was washed with acetone three times and dried under high vacuum to afford a pale white polymer (pCOEP-HCl). The polymer was redissolved in 5 mL CH₂Cl₂/methanol cosolvent (4:1 v/v) and was directly transferred into a glass solvent evaporation dish. The solvents were removed at 50° C. for 6 hours and 70° C. for 2 hours. The resultant membrane was then soaked in 1 M KOH (aq) for 24 hours, washed with water, and dried to afford pale white membrane (piperidine-functionalized poly(cyclooctene), pCOEP) which was subjected to ¹H NMR analysis in CD₂Cl₂ at 22° C.: ¹H NMR (400 MHz, CD₂Cl₂): δ 5.60-5.34 (br m, 10 H), 3.49-2.54 (br m, 5H), 2.47-1.67 (br m, 32 H), 1.66-0.89 (br m, 45 H).

To hydrogenate pCOEP-HCl, the unsaturated copolymer was dissolved in a 4:1 CH₂Cl₂/methanol cosolvent (10 mL) forming a yellow solution. The solution and Crabtree's catalyst (0.0020 mmol) were combined in a Parr reactor and sealed. It was pressurized to 40 atm hydrogen and then vented down to 3 atm. This process was repeated twice more to purge the reactor of air, then pressurized to 40 atm and heated to 100° C. with stirring. After 17 hours, it was cooled, vented and the slurry polymer mixture was dried under vacuum furnishing pale yellow solid which was subjected to ¹H NMR analysis in CDCl₃:CD₃OD=1:1 (v/v) at 22° C. Solvent residue peaks of dichloromethane and methanol were observed in this broad spectrum, while all alkene peaks disappeared after hydrogenation. The membrane was prepared in a Carver-Press (4120 Hydraulic Unit Carver press and stainless steel die molds, Teflon protective sheets from American Durafilm, 140° C. with 8,000 psig pressure for 5 min). The resultant membrane was then soaked in 1 M KOH (aq) for 24 hours, washed with water, and dried to afford yellow membrane PEP.

Alkaline Stabilities of Piperidinium Oligomers. General procedure: Alkaline stability study through ¹H NMR analysis was performed according to literature procedure. A solution of the basic methanol were prepared by dissolving KOH (2 M) and 3-(trimethylsilyl)-1-propanesulfonic acid sodium salt (NaDSS, 0.03 M) in CD₃OH. The oligomers (PEPM₅₂, PEPE₅₄, PEPB₅₈, PEPO₆₄, and PEQ55, estimated concentration of cationic units in solution was 0.03 M) and model compound 6 were dissolved in the methanol solution (0.5 mL) and passed through a glass wool plug into an NMR tube. The NMR tube was flame sealed and analyzed by ¹H NMR spectroscopy for the initial time point. Integration of a selected signal in the model compound relative to a signal related to NaDSS provided the initial quantity of model compound. The tube was heated in an oil bath at 80° C. At specified time points, every 5 days, the tubes were removed, cooled, and analyzed by ¹H NMR spectroscopy at 50° C. in order to determine the quantity of cation remaining in polymer.

Solvent suppression procedure: Quantitative ¹H NMR spectra for imidazolium monomer stability studies were acquired in CD₃OH at 50° C. The OH signal in CD₃OH was suppressed with a 2 second presaturation delay and continuous wave irradiation with decoupler field strength (γB1) of 138 Hz (equivalent to a presaturation power of 12). Spectra were acquired over a spectral width of −2 to 14 ppm with 20 second relaxation delay and nominal 90° excitation pulse. 32 scans were averaged for each analysis. Residual signals between 5.9-6.2 ppm derive from solvent suppression. The oligomer ¹H NMR stability data is summarized in Table 4 (data plots for FIGS. 14A-14B).

TABLE 4
Summary of piperidinium NMR stability
studies (FIGS. 14A-14B Data).

| | |
|---|---|
| Piperidinium | Cation Remaining (%)^{a} |

| Oligomers | 5 d | 10 d | 15 d | 20 d | 25 d | 30 d |
|---|---|---|---|---|---|---|
| Cation 6 | 98 | 97 | 96 | 95 | 95 | 94 |
| PEPM₅₂ | 93 | 86 | 78 | 73 | 66 | 59 |
| PEPE₅₄ | 81 | 72 | 65 | 53 | 43 | 31 |
| PEPB₅₈ | 87 | 68 | 40 | 25 | 18 | 13 |
| PEPO₆₄ | 74 | 36 | 28 | 21 | 14 | 7 |
| PEQ₅₅ | 95 | 90 | 86 | 80 | 72 | 65 |

Reaction Conditions: [Cation]:[KOH] = 1:67 in 2M KOH experiments at 80° C.
^{a}Percent of cation remaining, determined by ¹H NMR spectroscopy relative to an internal standard (NaDSS).

TABLE 5
Hydroxide conductivities of PEPMs at
various temperatures (FIG. 5B Data).

σ (OH⁻, 22° C.) (mS/cm)

| PEPM Membranes | 22° C. | 40° C. | 60° C. | 80° C. |
|---|---|---|---|---|
| PEPM₁₆ | 26 | 35 | 54 | 74 |
| PEPM₂₀ | 35 | 43 | 58 | 79 |
| PEPM₂₃ | 43 | 51 | 66 | 86 |
| PEPM₂₈ | 53 | 65 | 81 | 95 |

TABLE 6
Membrane conductivity stability in
1M KOH_{aq} at 80° C. (FIG. 7 Data).

σ (OH⁻, 22° C.) (mS/cm)

| AAEMs | 0 d | 3 d | 7 d | 15 d | 23 d | 30 d |
|---|---|---|---|---|---|---|
| PEPM₂₀ | 35 | 36 | 33 | 33 | 30 | 29 |
| PEPE₂₀ | 36 | 33 | 29 | 19 | 17 | 15 |
| PEPB₁₈ | 16 | 19 | 16 | 12 | 6 | 4 |
| PEPO₂₃ | 15 | 30 | 25 | 22 | 15 | 12 |
| PEQ₂₀ | 35 | 33 | 32 | 31 | 29 | 25 |

TABLE 7
Comparison of IEC determined by
¹H NMR analysis and Mohr titration.

| | Ionic | mol % | IEC | IEC |
|---|---|---|---|---|
| Samples | Monomer | of 1-5^{a} | (mmol I⁻/g)^{a} | (mmol I⁻/g)^{b} |
| PEPM₂₀ | 1 | 20% | 1.31 | 1.49 |
| PEPE₂₀ | 2 | 20% | 1.25 | 1.44 |
| PEPB₁₈ | 3 | 18% | 1.12 | 1.16 |
| PEPO₂₃ | 4 | 23% | 1.23 | 1.28 |
| PEQ₂₀ | 5 | 20% | 1.25 | 1.45 |

^{a}Determined by integration ratios in ¹H NMR spectra.
^{b}Determined by Mohr titration. The titration was performed by adapting literature procedures.⁹ The samples were first exchanged into the chloride form by soaking in 100 mL of 1M NaCl for 30 min, repeated three times with fresh solution, followed by washing with 100 mL of DI water for 30 min (min = minute(s)) three times. The Cl⁻form membranes were then dried for 8 hours under vacuum at 80° C. The dry, Cl⁻ form samples were weighed, followed by soaking in a 100 mL sample cup containing 60 mL of 0.1M NaNO₃ solution for a minimum of 6 hours. The solutions were then titrated with a 0.1M AgNO₃ solution using Na₂CrO₄ as the indicator to determinethe concentration of Cl⁻. The titrated IEC in the Cl⁻ form was then calculated according to the following equation to convert into the I⁻ form to compare with the NMR IEC values:
IEC(Cl⁻) = [Cl⁻]/(Weight of membrane in Cl⁻ form)
IEC(I⁻) = IEC[Cl⁻]/(1 + 0.015 × IEC(Cl⁻)).

TABLE 8
Weight loss of glass containers after alkaline stability studies.

| | Weight before | Weight after | |
|---|---|---|---|
| | Stability | Stability | Weight |
| Containers | Study (g) | Study (g) | Loss (g) |
| Soda Lime Vial^{a} | 13.1882 | 12.7048 | 0.4834 |
| Borosilicate NMR Tube | 2.5098 | 2.5098 | 0 |
| (Norell # S-5-600-7)^{b} | | | |

^{a}The conditions were the same as the membrane alkaline stability study conditions. A dried 20 mL soda lime glass vial was weighed and was then filled with 20 mL 1M KOH. The vial was then sealed and heated to 80° C. The vial was kept at 80° C. for 30 days while the KOH solution was refreshed every 5 days. After the study the vial was emptied, washed with DI water, and dried before measuring its final weight.
^{b}The conditions were the same as the NMR alkaline stability study conditions. A dry NMR tube was weighed and was then filled with a solution of KOH (2M), NaDSS (0.03M), and 18-crown-6 (0.03M as another common internal standard) in 1 mL CD3OH. The NMR tube was flame sealed. The tube was heated in an oil bath at 80° C. for 30 days. After the stability study, the tube was carefully cut open, emptied, washed, and dried. The tube was then weighed along with the glass pieces removed during flame-seal and cut-open processes.

Although the present disclosure has been described with respect to one or more particular example(s), it will be understood that other examples of the present disclosure may be made without departing from the scope of the present disclosure.

1. A compound comprising the following structure: 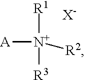
wherein
A is a cyclooctenyl group or a norbornenyl group;
R¹, R², and R³ are independently at each occurrence an aliphatic group, or
R¹, R², and N taken together form a heterocyclic group and R³ is an aliphatic group, or
R¹, R², R³, and N taken together form an aliphatic group-bridged heterocyclic group, or
R³ comprises a linking group and a 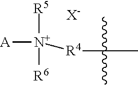
group, wherein a first terminus of the linking group is covalently bound to the quaternary ammonium nitrogen of the compound and a second terminus of the linking group is covalently bound the 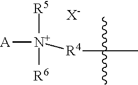
group through the R⁴ group, and R¹ and R² are independently at each occurrence an aliphatic group or R¹, R², and the N of the compound taken together form a heterocyclic group, and R⁵ and R⁶ are independently at each occurrence an aliphatic group or R⁵, R⁶, and the N of the 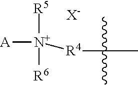
group taken together form a heterocyclic group, or
R¹ and/or R² each comprises a linking group and a 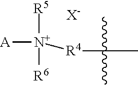
group, wherein a first terminus of the linking group is covalently bound to the quaternary ammonium nitrogen of the compound and a second terminus of the linking group is covalently bound the 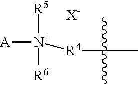
group through the R⁴ group, wherein the R⁴ group is an aliphatic group, R³ is an aliphatic group, and R⁵ and R⁶ are independently at each occurrence an aliphatic group or R⁵, R⁶, and the N of the 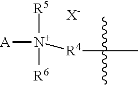
group taken together form a heterocyclic group, or
R¹, R², and N taken together form a heterocyclic linking group, wherein a first terminus of the heterocyclic linking group is covalently bound to the quaternary ammonium nitrogen of the compound and a second terminus of the heterocyclic linking group is covalently bound a 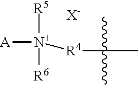
group through the R⁴ group, wherein the R⁴ group is an aliphatic group, R³ is an aliphatic group, and R⁵ and R⁶ are independently at each occurrence an aliphatic group or R⁵, R⁶, and the N of the 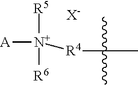
group taken together form a heterocyclic group, or
R¹, R², and N taken together form an aliphatic group-bridged heterocyclic linking group, wherein a first terminus of the linking bridged heterocyclic group is covalently bound to the quaternary ammonium nitrogen of the compound and a second terminus of the aliphatic group-bridged heterocyclic linking group is covalently bound a 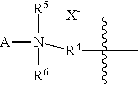
group, wherein the R⁴ group is an aliphatic group, R³ is an aliphatic group, and R⁵ and R⁶ are independently at each occurrence an aliphatic group or R⁵, R⁶, and the N of the 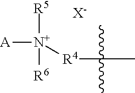
group taken together form a heterocyclic group; and
X is chosen independently at each occurrence from halide anions and complex anions.

2. The compound of claim 1, wherein the compound comprises the following structure:

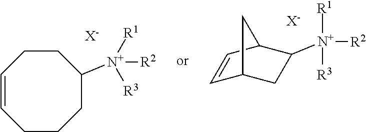

3. The compound of claim 1, wherein R¹ and R² taken together with N form:
a piperidinyl group, pyrrolidinyl group, azepanyl group, morpholinyl group, piperazinyl group, or a 1,4-Diazabicyclo[2.2.2]octane (DABCO) group.

4. The compound of claim 1, wherein R², and N taken together form a heterocyclic group, and wherein the compound comprises the following structure: 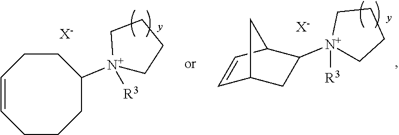
wherein y is 1, 2, 3, or 4.

5. The compound of claim 1, wherein R¹ and R² taken together with N form a piperidinium group or R¹, R², and R³ taken together with N form an aliphatic-bridged piperidinium group, and wherein the compound comprises the following structure:

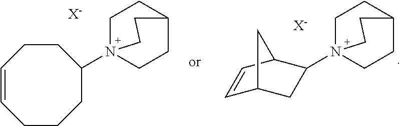

6. The compound of claim 1, wherein R¹ and R² taken together with N form a piperidinium group or R¹, R², and R³ taken together with N form an aliphatic-bridged heterocyclic group and wherein the compound comprises the following structure: 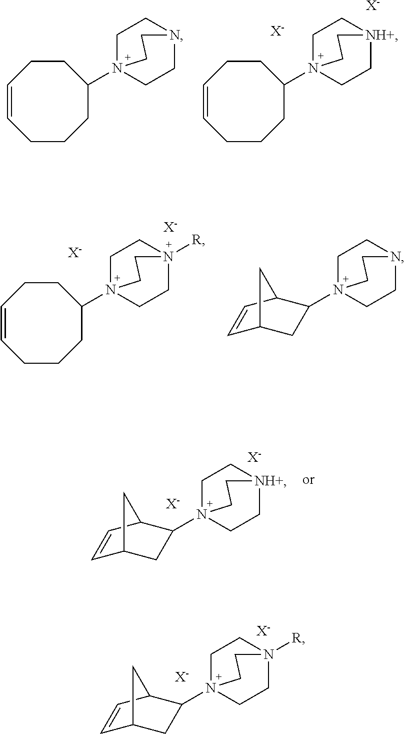
wherein R is an aliphatic group or an aryl group.

7. The compound of claim 1, wherein the compound has the following structure: 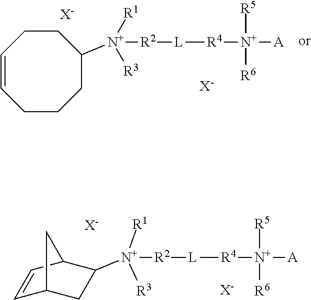
wherein L is a linking group and R⁴, R⁵, and R⁶ are independently at each occurrence an aliphatic group.

8. A method of making a compound of claim 1, the method comprising:
forming a first reaction mixture comprising:
1,5 cyclooctadiene, norbornadiene, or a combination thereof;
one or more secondary amine(s), one or more hydroxyalkyl secondary amine(s),
one or more multifunctional secondary amine(s) or any combination thereof;
one or more H-atom transfer (HAT) catalyst(s);
and one or more photocatalyst(s);
subjecting the first reaction mixture to electromagnetic radiation comprising wavelengths from about 350 nm to about 700 nm to form one or more tertiary amine-functionalized cyclooctene compound(s), one or more tertiary amine-functionalized norbornene compound(s), one or more functionalized tertiary amine-functionalized cyclooctene compound(s), one or more functionalized tertiary amine-functionalized norbornene compound(s), one or more hydroxyalkyl amine-functionalized cyclooctene compound(s), one or more hydroxyalkylamine-functionalized norbornene compound(s), or any combination thereof;
forming a second reaction mixture comprising:
the tertiary amine-functionalized cyclooctene compound(s) and/or the tertiary amine-functionalized cyclooctene compound(s) and/or the functionalized tertiary amine-functionalized norbornene compound(s), or any combination thereof;
one or more alkylating agent(s), or a combination thereof; or
the hydroxyalkyl amine-functionalized cyclooctene compound(s), the hydroxyalkylamine-functionalized norbornene compound(s), functionalized tertiary amine-functionalized norbornene compound(s), or any combination thereof,
one or more reductant(s); and
one or more bromine source(s),
wherein one or more compound(s) of claim 1 is formed.

9. The method of claim 8, further comprising one or more or all of the following:
isolating the tertiary amine-functionalized cyclooctene compound(s), the tertiary amine-functionalized norbornene compound(s), the functionalized tertiary amine-functionalized cyclooctene compound(s), the functionalized tertiary amine-functionalized norbornene compound(s) or any combination thereof, prior to formation of the second reaction mixture; or
isolating the tertiary amine-functionalized cyclooctene compound or the (bridged piperidinium)-functionalized cyclooctene compound from the second reaction mixture.

10. The method of claim 8, wherein the one or more secondary amine(s) are chosen from pyrrolidines, azepanes, morpholines, piperazines, piperidin-4-amine, piperidin-4-ol, 1,4-Diazabicyclo[2.2.2]octane (DABCO), secondary alkyl amines, N-alkyl-N-aryl amines, N-methylcyclohexanamine, 2,2-imethoxy-N-methylethan-1-amine, N-methyl-2-(pyridine-4-yl)ethan-1-amine.

11. The method of claim 8, wherein the secondary amines are multifunctional secondary amines.

12. The method of claim 11, wherein the secondary amines are chosen from
N(H)R⁵R⁶, wherein R⁵ and R⁶ are independently chosen from aliphatic groups, 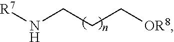
wherein R⁷ is an aliphatic group and R⁸ is H or an aliphatic group and n is 0 to 6, 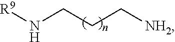
wherein R⁹ is an aliphatic group and n is 0 to 6, 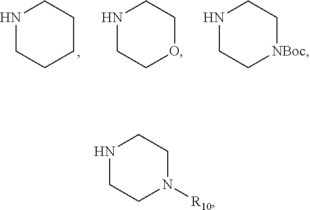
wherein R¹⁰ is an aliphatic group or an aryl group, 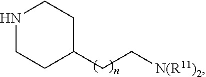
wherein the R″ groups are both —H groups or both aliphatic groups and n is 0 to 6, 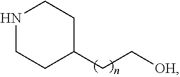
wherein n is 0 to 6, 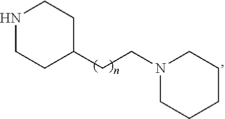
wherein n is 0 to 6, 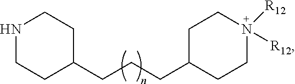
wherein n is 0 to 6 and R¹² is independently at each occurance an aliphatic group, 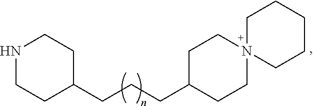
wherein n is 0 to 6, 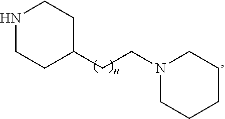
wherein n is 0 to 6; and 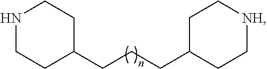
wherein n is 1 to 6;
and any combination thereof.

13. The method of claim 8, wherein the hydroxyalkyl secondary amine(s) are chosen from 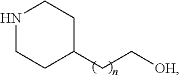
wherein n is 0 to 6, and any combination thereof and/or
the multifunctional secondary amine(s) are chosen from 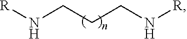
wherein n is 0 to 6 and is R independently, at each occurrence, is an aliphatic group, 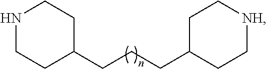
wherein n is 0 to 6.

14. The method of claim 8, wherein the 1,5-cyclooctadiene and/or norbornadiene are present at about 0.25 molar equivalents to about 50 molar equivalents, based on the total moles of secondary amine(s), one or more hydroxyalkyl amine(s), or any combination thereof.

15. The method of claim 8, wherein the secondary amine(s), the one or more hydroxyalkyl amine(s), or any combination thereof, are present at a concentration of from about 0.01 M to about 5 M (based on the total volume of the reaction mixture).

16. The method of claim 8, wherein the H-atom transfer (HAT) catalyst is chosen from thiophenol, substituted thiophenols, phenol, substituted phenols, thiols, malonitrile, hydroxamic acid, silanes, and any combinations thereof.

17. The method of claim 8, wherein the H-atom transfer (HAT) catalyst is present at about 0.1 mol % to about 500 mol %, based on the total moles of secondary amine(s), hydroxyalkyl amine(s), or any combination thereof.

18. The method of claim 8, wherein the photocatalyst is chosen from iridium-bipyridine photocatalysts, acridinium photocatalysts and any combination thereof.

19. The method of claim 8, wherein the photocatalyst is present at about 0.01 mol % to about 10 mol %, based on the total moles of pyridine, one or more hydroxyalkyl pyridine(s), or any combination thereof.

20. The method of claim 7, further comprising one or both of the following:
the first reaction mixture further comprises one or more solvent(s) chosen from non-polar solvents, cyclic ethers, aromatic solvents, aprotic solvents, and the like, and any combination thereof, or
the second reaction mixture comprises one or more solvent(s) chosen from cyclic ethers, chlorinated solvents, aprotic solvents, and the like, and any combination thereof.

21. The method of claim 8, wherein the first reaction mixture is subjected to the electromagnetic radiation for about 5 minutes to about 168 hours.

22. The method of claim 8, wherein the first reaction mixture is subjected to the electromagnetic radiation at a temperature of about −50° C. to about 110° C.

23. The method of claim 8, wherein the alkylating agent(s) is/are one or more hydrocarbon halide(s) is/are chosen from alkyl halides, wherein the halide group is independently at each occurrence iodide (—I), bromide (—Br), chloride (—Cl), or the like, trialkyloxonium salts, wherein the anion of the trialkyloxonium salt(s) is independently at each occurrence chosen from complex anions and the like, and any combination thereof.

24. The method of claim 8, wherein the alkyl halide(s) and/or trialkyloxonium salt(s) is/are present at about 0.1 to about 50 molar equivalents based on the total moles of precursor compound(s).

25. The method of claim 8, further comprising:
heating or holding the second reaction mixture to a temperature of from about −50° C. to about 120° C., when the second reaction mixture comprises the tertiary amine-functionalized cyclooctene compound(s) and/or the tertiary amine-functionalized cyclooctene compound(s) and/or the functionalized tertiary amine-functionalized cyclooctene compound(s), or any combination thereof, and the alkylating agent(s); or
cooling or holding the second reaction mixture to a temperature of from about −20° C. to about 80° C., when the second reaction mixture comprises the one or more hydroxyalkyl amine-functionalized cyclooctene compound(s), one or more hydroxyalkylamine-functionalized norbornene compound(s), or any combination thereof, the reductant(s), and bromine source(s).

26. A polymer comprising the following structure: 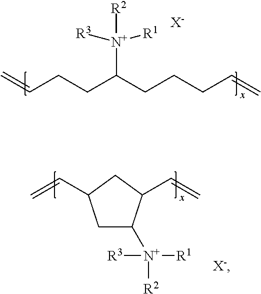
or any combination thereof, wherein
R¹, R², and R³ are independently at each occurrence an aliphatic group, or
R¹, R², and N taken together form a heterocyclic group and R³ is an aliphatic group, or
R¹, R², R³, and N taken together form an aliphatic group-bridged heterocyclic group, or
R³ comprises a linking group and a 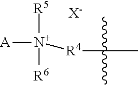
group, wherein a first terminus of the linking group is covalently bound to the quaternary ammonium nitrogen of the compound and a second terminus of the linking group is covalently bound the 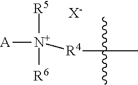
group through the R⁴ group, and R¹ and R² are independently at each occurrence an aliphatic group or R¹, R², and the N of the compound taken together form a heterocyclic group, and R⁵ and R⁶ are independently at each occurrence an aliphatic group or R⁵, R⁶, and the N of the 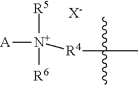
group taken together form a heterocyclic group, or R¹ and/or R² each comprises a linking group and a 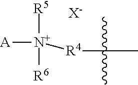
group, wherein a first terminus of the linking group is covalently bound to the quaternary ammonium nitrogen of the compound and a second terminus of the linking group is covalently bound the 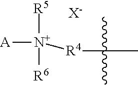
group through the R⁴ group, wherein the R⁴ group is an aliphatic group, R³ is an aliphatic group, and R⁵ and R⁶ are independently at each occurrence an aliphatic group or R⁵, R⁶, and the N of the 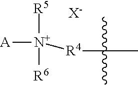
group taken together form a heterocyclic group, or
R¹, R², and N taken together form a heterocyclic linking group, wherein a first terminus of the heterocyclic linking group is covalently bound to the quaternary ammonium nitrogen of the compound and a second terminus of the heterocyclic linking group is covalently bound a 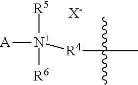
group through the R⁴ group, wherein the R⁴ group is an aliphatic group, R³ is an aliphatic group, and R⁵ and R⁶ are independently at each occurrence an aliphatic group or R⁵, R⁶, and the N of the 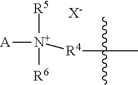
group taken together form a heterocyclic group, or
R¹, R², and N taken together form an aliphatic group-bridged heterocyclic linking group, wherein a first terminus of the linking bridged heterocyclic group is covalently bound to the quaternary ammonium nitrogen of the compound and a second terminus of the aliphatic group-bridged heterocyclic linking group is covalently bound a 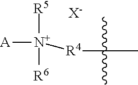
group, wherein the R⁴ group is an aliphatic group, R³ is an aliphatic group, and R⁵ and R⁶ are independently at each occurrence an aliphatic group or R⁵, R⁶, and the N of the 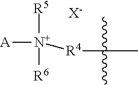
group taken together form a heterocyclic group; and
X is chosen independently at each occurrence from halide anions, complex anions, and any combination thereof; and
x is the mol fraction of structure(s) in the polymer and the mol fraction of structures is about 0.01 to about 1.

27. The polymer of claim 26, wherein the polymer comprises the following structure:

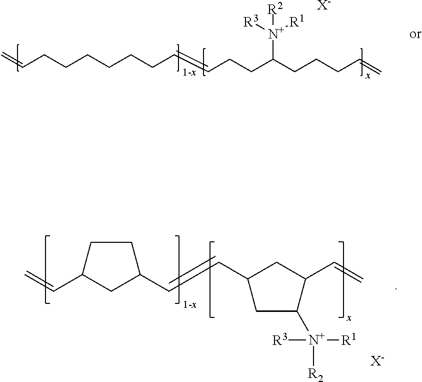

28. The polymer of claim 26, wherein the polymer has a molecular weight (Mw and/or Mn) of 500 g/mol to 1,000,000 g/mol.

29. The polymer of claim 26, wherein at least a portion of the terminal carbons is, independently at each occurrence, substituted with an aryl group, hydrogen, an alkyl group, a halogen, a hydroxyl group, and/or at least a portion of the terminal carbons is, independently at each occurrence, a carbonyl carbon of an aldehyde group, a ketone group, an acid group, or acetate group.

30. A polymer comprising the following structure: 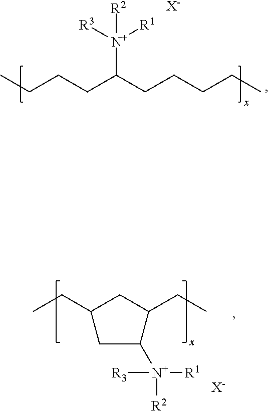
or any combination thereof, wherein
R¹, R², and R³ are independently at each occurrence an aliphatic group, or
R¹, R², and N taken together form a heterocyclic group and R³ is an aliphatic group, or
R¹, R², R³, and N taken together form an aliphatic group-bridged heterocyclic group, or
R³ comprises a linking group and a 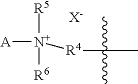
group, wherein a first terminus of the linking group is covalently bound to the quaternary ammonium nitrogen of the compound and a second terminus of the linking group is covalently bound the 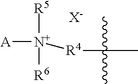
group through the R⁴ group, and R¹ and R² are independently at each occurrence an aliphatic group or R¹, R², and the N of the compound taken together form a heterocyclic group, and R⁵ and R⁶ are independently at each occurrence an aliphatic group or R⁵, R⁶, and the N of the 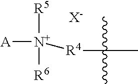
group taken together form a heterocyclic group, or R¹ and/or R² each comprises a linking group and a 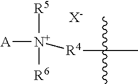
group, wherein a first terminus of the linking group is covalently bound to the quaternary ammonium nitrogen of the compound and a second terminus of the linking group is covalently bound the 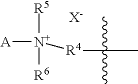
group through the R⁴ group, wherein the R⁴ group is an aliphatic group, R³ is an aliphatic group, and R⁵ and R⁶ are independently at each occurrence an aliphatic group or R⁵, R⁶, and the N of the 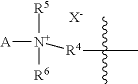
group taken together form a heterocyclic group, or
R¹, R², and N taken together form a heterocyclic linking group, wherein a first terminus of the heterocyclic linking group is covalently bound to the quaternary ammonium nitrogen of the compound and a second terminus of the heterocyclic linking group is covalently bound a 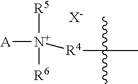
group through the R⁴ group, wherein the R⁴ group is an aliphatic group, R³ is an aliphatic group, and R⁵ and R⁶ are independently at each occurrence an aliphatic group or R⁵, R⁶, and the N of the 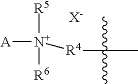
group taken together form a heterocyclic group, or
R¹, R², and N taken together form an aliphatic group-bridged heterocyclic linking group, wherein a first terminus of the linking bridged heterocyclic group is covalently bound to the quaternary ammonium nitrogen of the compound and a second terminus of the aliphatic group-bridged heterocyclic linking group is covalently bound a 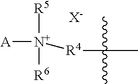
group, wherein the R⁴ group is an aliphatic group, R³ is an aliphatic group, and R⁵ and R⁶ are independently at each occurrence an aliphatic group or R⁵, R⁶, and the N of the 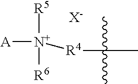
group taken together form a heterocyclic group; and
X is chosen independently at each occurrence from halide anions, complex anions optionally chosen from BF₄⁻, SbF₆⁻, PF₆⁻, and B(ArF₄)⁻, and any combination thereof; and
x is the mol fraction of the structure(s) (repeat unit(s)) in the polymer and the mol fraction of structure(s) (repeat unit(s)) is/are about 0.01 to about 1.

31. The polymer of claim 30, wherein the polymer comprises the following structure:

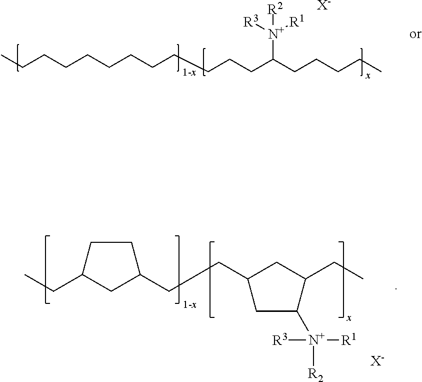

32. The polymer of claim 29, wherein the polymer has a molecular weight (Mw and/or Mn) of 500 g/mol to 1,000,000 g/mol.

33. The polymer of claim 29, wherein at least a portion of the terminal carbons is, independently at each occurrence, substituted with an aryl group, hydrogen, an alkyl group, a halogen, a hydroxyl group, and/or at least a portion of the terminal carbons is, independently at each occurrence, a carbonyl carbon of an aldehyde group, a ketone group, an acid group, or acetate group.

34. A method of making a polymer comprising following structure: 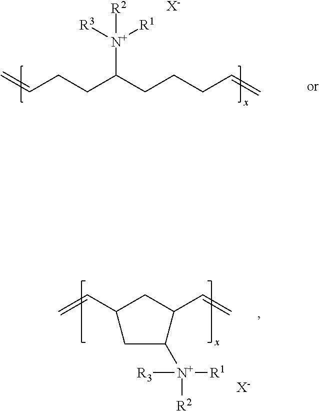
or any combination thereof, wherein
R¹, R², and R³ are independently at each occurrence an aliphatic group, or
R¹, R², and N taken together form a heterocyclic group and R³ is an aliphatic group, or
R¹, R², R³, and N taken together form an aliphatic group-bridged heterocyclic group, or
R³ comprises a linking group and a 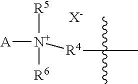
group, wherein a first terminus of the linking group is covalently bound to the quaternary ammonium nitrogen of the compound and a second terminus of the linking group is covalently bound the 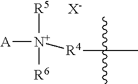
group through the R⁴ group, and R¹ and R² are independently at each occurrence an aliphatic group or R¹, R², and the N of the compound taken together form a heterocyclic group, and R⁵ and R⁶ are independently at each occurrence an aliphatic group or R⁵, R⁶, and the N of the 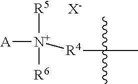
group taken together form a heterocyclic group, or
R¹ and/or R² each comprises a linking group and a 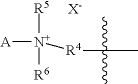
group, wherein a first terminus of the linking group is covalently bound to the quaternary ammonium nitrogen of the compound and a second terminus of the linking group is covalently bound the 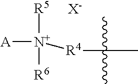
group through the R⁴ group, wherein the R⁴ group is an aliphatic group, R³ is an aliphatic group, and R⁵ and R⁶ are independently at each occurrence an aliphatic group or R⁵, R⁶, and the N of the 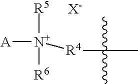
group taken together form a heterocyclic group, or
R¹, R², and N taken together form a heterocyclic linking group, wherein a first terminus of the heterocyclic linking group is covalently bound to the quaternary ammonium nitrogen of the compound and a second terminus of the heterocyclic linking group is covalently bound a 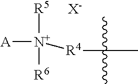
group through the R⁴ group, wherein the R⁴ group is an aliphatic group, R³ is an aliphatic group, and R⁵ and R⁶ are independently at each occurrence an aliphatic group or R⁵, R⁶, and the N of the 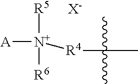
group taken together form a heterocyclic group, or
R¹, R², and N taken together form an aliphatic group-bridged heterocyclic linking group, wherein a first terminus of the linking bridged heterocyclic group is covalently bound to the quaternary ammonium nitrogen of the compound and a second terminus of the aliphatic group-bridged heterocyclic linking group is covalently bound a 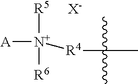
group, wherein the R⁴ group is an aliphatic group, R³ is an aliphatic group, and R⁵ and R⁶ are independently at each occurrence an aliphatic group or R⁵, R⁶, and the N of the 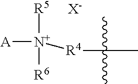
group taken together form a heterocyclic group; and
X is chosen independently at each occurrence from halide anions, complex anions optionally chosen from BF₄⁻, SbF₆⁻, PF₆⁻, and B(ArF₄)⁻, and any combination thereof, and x is the mol fraction of the structure(s) (repeat unit(s)) in the polymer and the mol fraction of structure(s) (repeat unit(s)) is/are about 0.01 to about 1, the method comprising
polymerizing one or more monomer(s) independently chosen from monomer(s) having the following structure: 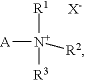
wherein
A is a cyclooctenyl group or a norbornenyl group;
R¹, R², and R³ are independently at each occurrence an aliphatic group, or
R¹ and R² are taken together with N to form a heterocyclic group and R³ is an aliphatic group, or R¹, R², and R³ are taken together with N to form bridged heterocyclic group; or
or
R³ comprises a linking group and a 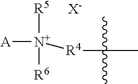
group, wherein a first terminus of the linking group is covalently bound to the quaternary ammonium nitrogen of the compound and a second terminus of the linking group is covalently bound the 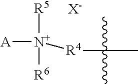
group through the R⁴ group, and R¹ and R² are independently at each occurrence an aliphatic group or R¹, R², and the N of the compound taken together form a heterocyclic group, and R⁵ and R⁶ are independently at each occurrence an aliphatic group or R⁵, R⁶, and the N of the 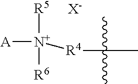
group taken together form a heterocyclic group, or
R¹ and/or R² each comprises a linking group and a 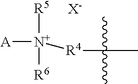
group, wherein a first terminus of the linking group is covalently bound to the quaternary ammonium nitrogen of the compound and a second terminus of the linking group is covalently bound the 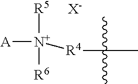
group through the R⁴ group, wherein the R⁴ group is an aliphatic group, R³ is an aliphatic group, and R⁵ and R⁶ are independently at each occurrence an aliphatic group or R⁵, R⁶, and the N of the 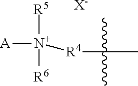
group taken together form a heterocyclic group, or
R¹, R², and N taken together form a heterocyclic linking group, wherein a first terminus of the heterocyclic linking group is covalently bound to the quaternary ammonium nitrogen of the compound and a second terminus of the heterocyclic linking group is covalently bound a 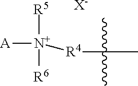
group through the R⁴ group, wherein the R⁴ group is an aliphatic group, R³ is an aliphatic group, and R⁵ and R⁶ are independently at each occurrence an aliphatic group or R⁵, R⁶, and the N of the 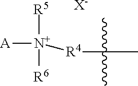
group taken together form a heterocyclic group, or
R¹, R², and N taken together form an aliphatic group-bridged heterocyclic linking group, wherein a first terminus of the linking bridged heterocyclic group is covalently bound to the quaternary ammonium nitrogen of the compound and a second terminus of the aliphatic group-bridged heterocyclic linking group is covalently bound a 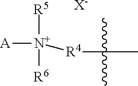
group, wherein the R⁴ group is an aliphatic group, R³ is an aliphatic group, and R⁵ and R⁶ are independently at each occurrence an aliphatic group or R⁵, R⁶, and the N of the 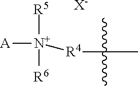
group taken together form a heterocyclic group; and
X is chosen from halides, BF₄⁻, SbF₆⁻, PF₆⁻, B(ArF₄)⁻, wherein ArF₄ is an aryl group substituted with four fluorine groups, B(Ar₄)⁻, wherein Ar is an aryl group,
or
copolymerizing one or more monomer(s) independently chosen from monomer(s) having the following structure: 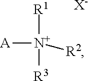
wherein
A is a cyclooctenyl group or a norbornenyl group; R¹, R², and R³ are independently at each occurrence an aliphatic group, or
R¹, R², and N taken together form a heterocyclic group and R³ is an aliphatic group, or
R¹, R², R³, and N taken together form an aliphatic group-bridged heterocyclic group, or
or
R³ comprises a linking group and a 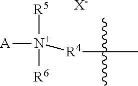
group, wherein a first terminus of the linking group is covalently bound to the quaternary ammonium nitrogen of the compound and a second terminus of the linking group is covalently bound the 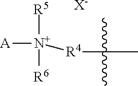
group through the R⁴ group, and R¹ and R² are independently at each occurrence an aliphatic group or R¹, R², and the N of the compound taken together form a heterocyclic group, and R⁵ and R⁶ are independently at each occurrence an aliphatic group or R⁵, R⁶, and the N of the 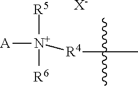
group taken together form a heterocyclic group, or
R¹ and/or R² each comprises a linking group and a 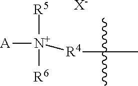
group, wherein a first terminus of the linking group is covalently bound to the quaternary ammonium nitrogen of the compound and a second terminus of the linking group is covalently bound the 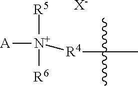
group through the R⁴ group, wherein the R⁴ group is an aliphatic group, R³ is an aliphatic group, and R⁵ and R⁶ are independently at each occurrence an aliphatic group or R⁵, R⁶, and the N of the 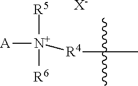
group taken together form a heterocyclic group, or
R¹, R², and N taken together form a heterocyclic linking group, wherein a first terminus of the heterocyclic linking group is covalently bound to the quaternary ammonium nitrogen of the compound and a second terminus of the heterocyclic linking group is covalently bound a 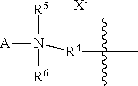
group through the R⁴ group, wherein the R⁴ group is an aliphatic group, R³ is an aliphatic group, and R⁵ and R⁶ are independently at each occurrence an aliphatic group or R⁵, R⁶, and the N of the 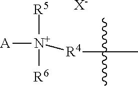
group taken together form a heterocyclic group, or
R¹, R², and N taken together form an aliphatic group-bridged heterocyclic linking group, wherein a first terminus of the linking bridged heterocyclic group is covalently bound to the quaternary ammonium nitrogen of the compound and a second terminus of the aliphatic group-bridged heterocyclic linking group is covalently bound a 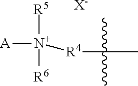
group, wherein the R⁴ group is an aliphatic group, R³ is an aliphatic group, and R⁵ and R⁶ are independently at each occurrence an aliphatic group or R⁵, R⁶, and the N of the 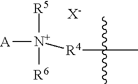
group taken together form a heterocyclic group; and
X is chosen independently at each occurrence from halide anions, complex anions optionally chosen from BF₄⁻, SbF₆⁻, PF₆⁻, and B(ArF₄)⁻, and any combination thereof; and one or more co-monomer(s) is/are chosen from unsaturated hydrocarbon monomers.

35. The method of claim 34, wherein the polymer comprises the following structure:

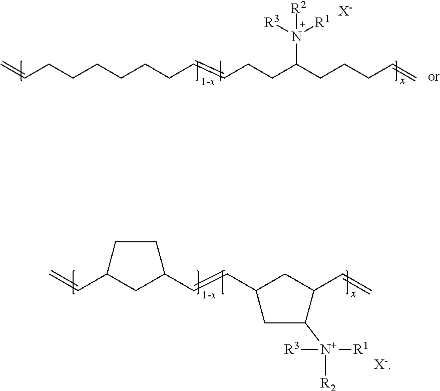

36. The method of claim 34, further comprising at least partially, substantially, or completely hydrogenating the polymer.

37. The method of claim 36, wherein the hydrogenated polymer comprises the following structure:

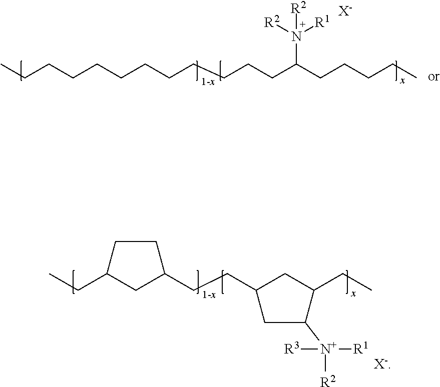

38. An anion exchange membrane comprising one or more polymer(s) comprising the following structure: 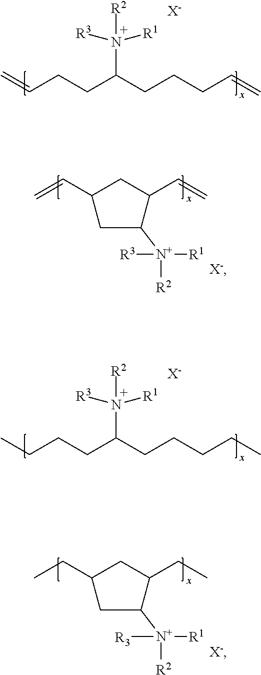
or any combination thereof,
wherein
R¹, R², and R³ are independently at each occurrence an aliphatic group, or
R¹, R², and N taken together form a heterocyclic group and R³ is an aliphatic group, or
R¹, R², R³, and N taken together form an aliphatic group-bridged heterocyclic group, or
R³ comprises a linking group and a 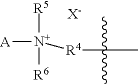
group, wherein a first terminus of the linking group is covalently bound to the quaternary ammonium nitrogen of the compound and a second terminus of the linking group is covalently bound the 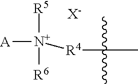
group through the R⁴ group, and R¹ and R² are independently at each occurrence an aliphatic group or R¹, R², and the N of the compound taken together form a heterocyclic group, and R⁵ and R⁶ are independently at each occurrence an aliphatic group or R⁵, R⁶, and the N of the 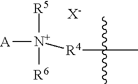
group taken together form a heterocyclic group, or
R¹ and/or R² each comprises a linking group and a 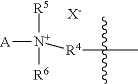
group, wherein a first terminus of the linking group is covalently bound to the quaternary ammonium nitrogen of the compound and a second terminus of the linking group is covalently bound the 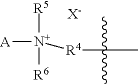
group through the R⁴ group, wherein the R⁴ group is an aliphatic group, R³ is an aliphatic group, and R⁵ and R⁶ are independently at each occurrence an aliphatic group or R⁵, R⁶, and the N of the 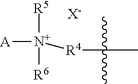
group taken together form a heterocyclic group, or
R¹, R², and N taken together form a heterocyclic linking group, wherein a first terminus of the heterocyclic linking group is covalently bound to the quaternary ammonium nitrogen of the compound and a second terminus of the heterocyclic linking group is covalently bound a 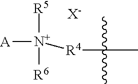
group through the R⁴ group, wherein the R⁴ group is an aliphatic group, R³ is an aliphatic group, and R⁵ and R⁶ are independently at each occurrence an aliphatic group or R⁵, R⁶, and the N of the 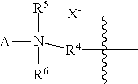
group taken together form a heterocyclic group, or
R¹, R², and N taken together form an aliphatic group-bridged heterocyclic linking group, wherein a first terminus of the linking bridged heterocyclic group is covalently bound to the quaternary ammonium nitrogen of the compound and a second terminus of the aliphatic group-bridged heterocyclic linking group is covalently bound a 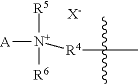
group, wherein the R⁴ group is an aliphatic group, R³ is an aliphatic group, and R⁵ and R⁶ are independently at each occurrence an aliphatic group or R⁵, R⁶, and the N of the 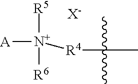
group taken together form a heterocyclic group; and
X is chosen independently at each occurrence from halide anions, complex anions optionally chosen from BF₄⁻, SbF₆⁻, PF₆⁻, and B(ArF₄)⁻, and any combination thereof; and
x is the mol fraction of the structure(s) in the polymer and the mol fraction of structure(s) is/are about 0.01 to about 1.

39. The anion exchange membrane of claim 38, wherein the one or more polymer(s) comprising the following structure:

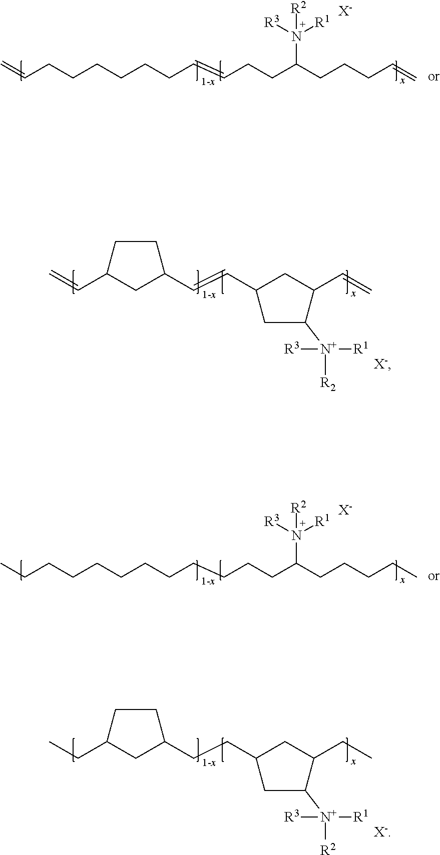

40. The anion exchange membrane of claim 38, wherein the membrane has a thickness of about 1 micron to about 500 microns.

41. The anion exchange membrane of claim 38, wherein the membrane exhibits one or more or all of the following:
a hydroxide conductivity (σ(OH—, at about 22° C.)) of from about 1 mS/cm to about 60 mS/cm;
a water uptake (WU) of from about 5% to about 200%;
a dimensional change (ΔL) of from about 0% to about 30%;
an ion exchange capacities (IECs) of from about 0.08 mmol I⁻/g to about 3 mmol I⁻/g;
a stress at break of from about 1 MPa to about 30 MPa;
a strain at break of from about 100% to about 1600%; or
a retained conductivity of from about 0% to about 100%.

42. The anion exchange membrane of claim 38, wherein the membrane is formed by a method comprising crosslinking/reactive casting, solution casting, annealing, meltpress, or the like.

43. A device comprising one or more anion exchange membrane(s) of claim 38.

44. The device of claim 43, wherein the device is an energy-storage device, an energy-generating device, or the like.

45. The device of claim 44, wherein the electrochemical device is a battery, a fuel cell, a water-electrolysis device, or the like.
